SEQUENCE LISTING (72 sequences)

SEQ ID NO: 1 - DNA, 792 bp, Collinsella aerofaciens
  source 1..792: mol_type = genomic DNA
  CDS 1..789: protein_id = 70; translation = MNLREKYGEWGLILGATEGVGKAFCEKIAAGGMNVVMVGRREEK LNVLAGEIRETYGVETKVVRADFSQPGAAETVFAATEGLDMGFMSYVACLHSFGKIQD TPWEKHEAMINVNVVTFLKCFHHYMRIFAAQDRGAVINVSSMTGISSSPWNGQYGAGK AFILKMTEAVACECEGTGVDVEVITLGTTLTPSLLSNLPGGPQGEAVMKIALTPEECV DEAFEKLGKELSVIAGQRNKDSVHDWKANHTEDEYIRYMGSFYRD
atgaacctgagggagaagtacggtgagtggggcctgatcctgggcgcgaccgagggcgtcggcaaggcgttctgcgagaagatcgccgccggcggcatgaacgtcgtcatggtcggccgtcgcgaggagaagctgaacgtgctcgcaggcgagatccgcgagacctacggcgtggagaccaaggtcgtgcgcgccgactttagccagcccggcgctgccgagaccgtcttcgccgcgaccgagggcctggacatgggcttcatgagctacgtggcctgcctgcacagcttcggtaagatccaggacaccccctgggagaagcacgaggccatgatcaacgtcaacgtcgtgaccttcctcaagtgcttccaccactacatgcggatctttgccgcccaggaccgcggcgccgtgatcaacgtctcgtcgatgaccggcatcagctccagcccctggaacggccagtacggcgcgggcaaggccttcatcctcaagatgaccgaggccgtggcctgcgagtgcgagggcaccggcgtcgacgtcgaggtcatcaccctcggcaccaccctaacccccagcctgctgtccaacctccccggcggcccgcagggcgaggccgtcatgaagatcgccctcacccccgaggagtgcgttgacgaggcctttgagaagctgggtaaggagctctccgtcatcgccggccagcgcaacaaggactccgtccacgactggaaggcaaaccacaccgaggacgagtacatccgctacatggggtcgttctaccgcgactag

SEQ ID NO: 2 - protein, 263 aa, Collinsella aerofaciens
MNLREKYGEWGLILGATEGVGKAFCEKIAAGGMNVVMVGRREEKLNVLAGEIRETYGVETKVVRADFSQPGAAETVFAATEGLDMGFMSYVACLHSFGKIQDTPWEKHEAMINVNVVTFLKCFHHYMRIFAAQDRGAVINVSSMTGISSSPWNGQYGAGKAFILKMTEAVACECEGTGVDVEVITLGTTLTPSLLSNLPGGPQGEAVMKIALTPEECVDEAFEKLGKELSVIAGQRNKDSVHDWKANHTEDEYIRYMGSFYRD

SEQ ID NO: 3 - DNA, 31 bp, synthetic construct
  misc_feature 1..31: note = Description of Artificial Sequence: Synthetic PCR primer
  source 1..31: mol_type = other DNA
gggaattccatatgaacctgagggagaagta

SEQ ID NO: 4 - DNA, 27 bp, synthetic construct
  misc_feature 1..27: note = Description of Artificial Sequence: Synthetic PCR primer
  source 1..27: mol_type = other DNA
cccaagcttctagtcgcggtagaacga

SEQ ID NO: 5 - DNA, 774 bp, Comamonas testosteroni
  source 1..774: mol_type = genomic DNA
  CDS 1..771: protein_id = 71
atgtccatcatcgtgataagcggctgcgccaccggcattggtgccgctacgcgcaaggtcctggaggcggccggtcaccagatcgtaggcatcgatatacgcgatgcggaagtgattgccgatctctcgacggccgaaggtcgaaagcaggcgattgccgatgtactggcgaagtgcagcaagggcatggacggcctggtgctgtgcgccggcctgggaccgcagaccaaggtgcttggcaatgtggtttcggtcaattattttggcgcgaccgagctgatggatgcctttttgccagcgctgaaaaaaggccatcagcccgcagccgtcgtcatctcgtccgtggcttccgcgcatctggcttttgacaagaacccactggcgctggcactggaagccggcgaggaagccaaggcccgcgccattgtcgaacatgcgggagagcagggcggaaatctggcctatgcgggcagcaagaatgctttgacggtggctgtgcgcaaacgcgccgccgcctggggcgaggctggcgtgcgcctgaacaccatcgcccccggtgcaaccgagactcccttgctgcaggcgggcctgcaggacccgcgctatggcgaatccattgccaagttcgttcctcccatgggccgccgtgccgagccgtccgagatggcgtcggtcatcgcctttttgatgagcccggccgcaagctatgtgcatggcgcgcagatcgtcattgatggcggcattgatgcggtgatgcgcccgacacagttctga

SEQ ID NO: 6 - protein, 257 aa, Comamonas testosteroni [sequence omitted]

SEQ ID NO: 7 - DNA, 969 bp, Rattus norvegicus
  source 1..969: mol_type = genomic DNA
  CDS 1..966: protein_id = 72; translation = MDSISLRVALNDGNFIPVLGFGTTVPEKVAKDEVIKATKIAIDN GFRHFDSAYLYEVEEEVGQAIRSKIEDGTVKREDIFYTSKLWSTFHRPELVRTCLEKT LKSTQLDYVDLYIIHFPMALQPGDIFFPRDEHGKLLFETVDICDTWEAMEKCKDAGLA KSIGVSNFNCRQLERILNKPGLKYKPVCNQVECHLYLNQSKMLDYCKSKDIILVSYCT LGSSRDKTWVDQKSPVLLDDPVLCAIAKKYKQTPALVALRYQLQRGVVPLIRSFNAKR IKELTQVFEFQLASEDMKALDGLNRNFRYNNAKYFDDHPNHPFTDE
atggattccatatctctgcgtgtagcactaaatgatggtaacttcattcctgtactggggtttggaaccactgtgcctgagaaggttgctaaggatgaagttatcaaggctactaaaatagctatagataatggattccgccattttgactctgcttatttgtacgaagtagaagaggaagtgggccaagccattagaagcaagattgaagacggcactgtgaagagagaagatatattctatacttcaaagctttggagcactttccatagaccagagctggtccgaacttgcttggaaaagacactgaaaagcactcaactggactatgtggatctttatattattcatttcccaatggctttgcagcctggagatatatttttcccacgagatgagcatggaaaactattgtttgaaacagtggatatctgtgacacatgggaggccatggaaaagtgtaaggatgcaggattggccaagtctattggggtgtccaactttaactgcaggcagctggagaggattctgaataagccagggctcaaatacaagcctgtgtgcaaccaggtggaatgtcacctttatctcaaccagagcaaaatgctggactattgtaagtcaaaagacatcattctggtttcctactgcacgctgggaagttcacgagacaaaacatgggtggatcagaaaagtccagttctcctagatgatccagttctttgtgccatagcaaagaagtacaagcaaaccccagccctagttgcccttcgctaccagctgcagcgtggggttgtgcccctgatcaggagtttcaacgcgaagcggatcaaagagctaacacaggtttttgaattccagttggcttcagaggacatgaaagccctggatggcttgaacagaaatttcagatacaacaatgcaaaatattttgatgaccatcccaatcatccatttactgatgaatag

SEQ ID NO: 8 - protein, 322 aa, Rattus norvegicus
MDSISLRVALNDGNFIPVLGFGTTVPEKVAKDEVIKATKIAIDNGFRHFDSAYLYEVEEEVGQAIRSKIEDGTVKREDIFYTSKLWSTFHRPELVRTCLEKTLKSTQLDYVDLYIIHFPMALQPGDIFFPRDEHGKLLFETVDICDTWEAMEKCKDAGLAKSIGVSNFNCRQLERILNKPGLKYKPVCNQVECHLYLNQSKMLDYCKSKDIILVSYCTLGSSRDKTWVDQKSPVLLDDPVLCAIAKKYKQTPALVALRYQLQRGVVPLIRSFNAKRIKELTQVFEFQLASEDMKALDGLNRNFRYNNAKYFDDHPNHPFTDE

SEQ ID NO: 9 - DNA, 26 bp, synthetic construct
  misc_feature 1..26: note = Description of Artificial Sequence: Synthetic PCR primer
  source 1..26: mol_type = other DNA
cgtcgtcatggtcgcccgtcgcgagg

SEQ ID NO: 10 - DNA, 26 bp, synthetic construct
  misc_feature 1..26: note = Description of Artificial Sequence: Synthetic PCR primer
  source 1..26: mol_type = other DNA
cctcgcgacgggcgaccatgacgacg

SEQ ID NO: 11 - DNA, 26 bp, synthetic construct
  misc_feature 1..26: note = Description of Artificial Sequence: Synthetic PCR primer
  source 1..26: mol_type = other DNA
cgtcgtcatggtcagccgtcgcgagg

SEQ ID NO: 12 - DNA, 26 bp, synthetic construct
  misc_feature 1..26: note = Description of Artificial Sequence: Synthetic PCR primer
  source 1..26: mol_type = other DNA
cctcgcgacggctgaccatgacgacg

SEQ ID NO: 13 - DNA, 47 bp, synthetic construct
  misc_feature 1..47: note = Description of Artificial Sequence: Synthetic PCR primer
  source 1..47: mol_type = other DNA
gcgaattcgtgaaaggagatatacatgaacctgagggagaagtacgg

SEQ ID NO: 14 - DNA, 1206 bp, synthetic construct
  misc_feature 1..1206: note = Description of Artificial Sequence: Synthetic formate dehydrogenase mutant polynucleotide
  source 1..1206: mol_type = other DNA
  CDS 1..1203
atggcaaaggtcctgtgcgttctttacgatgatccggtcgacggctacccgaagacctatgcccgcgacgatcttccgaagatcgaccactatccgggcggccagatcttgccgacgccgaaggccatcgacttcacgcccgggcagttgctcggctccgtctccggcgagctcggcctgcgcgaatatctcgaatccaacggccacaccctggtcgtgacctccgacaaggacggccccgactcggtgttcgagcgcgagctggtcgatgcggatgtcgtcatctcccagcccttctggccggcctatctgacgcccgagcgcatcgccaaggccaagaacctgaagctcgcgctcaccgccggcatcggttccgaccacgtcgatcttcagtcggctatcgaccgcaacgtcaccgtggcggaagtcacctactgcaactcgatcagcgtcgccgagcatgtggtgatgatgatcctgtcgctggtgcgcaactatctgccctcgcacgaatgggcgcggaagggcggctggaacatcgccgactgcgtctcccacgcctacgacctcgaggcgatgcatgtcggcaccgtggccgccggccgcatcggtctcgcggtgctgcgccgtctggcgccgttcgacgtgcacctgcactacaccggccgtcaccgcctgccggaatcggtcgagaaggagctcaacctcacctggcacgcgacccgcgaggacatgtatccggtttgcgacgtggtgacgctgaactgcccgctgcaccccgaaaccgagcacatgatcaatgacgagacgctgaagctgttcaagcgtggcgcctacatcgtcaacaccgcccgcggcaagctgtgcgaccgcgatgccgtggcacgtgcgctcgaatccggccggctggccggctatgccggcgacgtgtggttcccgcagccggcgccgaaggaccacccctggcggacgatgccctataacggcatgaccccgcacatctccggcaccacgctgaccgcgcaggcgcgttatgcggcgggcacccgcgagatcctggagtgcttcttcgagggccgtccgatccgcgacgaatacctcatcgtgcagggcggcgctcttgccggcaccggcgcgcattcctactcgaagggcaatgccaccggcggttcggaagaggccgccaagttcaagaaggcggtctga

SEQ ID NO: 15 - protein, 401 aa, synthetic construct
  REGION 1..401: note = Description of Artificial Sequence: Synthetic construct polypeptide
MAKVLCVLYDDPVDGYPKTYARDDLPKIDHYPGGQILPTPKAIDFTPGQLLGSVSGELGLREYLESNGHTLVVTSDKDGPDSVFERELVDADVVISQPFWPAYLTPERIAKAKNLKLALTAGIGSDHVDLQSAIDRNVTVAEVTYCNSISVAEHVVMMILSLVRNYLPSHEWARKGGWNIADCVSHAYDLEAMHVGTVAAGRIGLAVLRRLAPFDVHLHYTGRHRLPESVEKELNLTWHATREDMYPVCDVVTLNCPLHPETEHMINDETLKLFKRGAYIVNTARGKLCDRDAVARALESGRLAGYAGDVWFPQPAPKDHPWRTMPYNGMTPHISGTTLTAQARYAAGTREILECFFEGRPIRDEYLIVQGGALAGTGAHSYSKGNATGGSEEAAKFKKAV

SEQ ID NO: 16 - DNA, 43 bp, synthetic construct
  misc_feature 1..43: note = Description of Artificial Sequence: Synthetic PCR primer
  source 1..43: mol_type = other DNA
cccaagcttaaggagatatacatgtccatcatcgtgataagcg

SEQ ID NO: 17 - DNA, 34 bp, synthetic construct
  misc_feature 1..34: note = Description of Artificial Sequence: Synthetic PCR primer
  source 1..34: mol_type = other DNA
ataagaatgcggccgctcagaactgtgtcgggcg

SEQ ID NO: 18 - DNA, 7404 bp, synthetic construct
  misc_feature 1..7404: note = Description of Artificial Sequence: Synthetic plasmid pET21a FDH D221G 7beta-HSDH polypeptide
  source 1..7404: mol_type = other DNA
  CDS 5208..6422: note = FDH D221G
  CDS 6435..7226: note = 7beta-HSDH
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatggcaaaggtcctgtgcgttctttacgatgatccggtcgacggctacccgaagacctatgcccgcgacgatcttccgaagatcgaccactatccgggcggccagatcttgccgacgccgaaggccatcgacttcacgcccgggcagttgctcggctccgtctccggcgagctcggcctgcgcgaatatctcgaatccaacggccacaccctggtcgtgacctccgacaaggacggccccgactcggtgttcgagcgcgagctggtcgatgcggatgtcgtcatctcccagcccttctggccggcctatctgacgcccgagcgcatcgccaaggccaagaacctgaagctcgcgctcaccgccggcatcggttccgaccacgtcgatcttcagtcggctatcgaccgcaacgtcaccgtggcggaagtcacctactgcaactcgatcagcgtcgccgagcatgtggtgatgatgatcctgtcgctggtgcgcaactatctgccctcgcacgaatgggcgcggaagggcggctggaacatcgccgactgcgtctcccacgcctacgacctcgaggcgatgcatgtcggcaccgtggccgccggccgcatcggtctcgcggtgctgcgccgtctggcgccgttcgacgtgcacctgcactacaccggccgtcaccgcctgccggaatcggtcgagaaggagctcaacctcacctggcacgcgacccgcgaggacatgtatccggtttgcgacgtggtgacgctgaactgcccgctgcaccccgaaaccgagcacatgatcaatgacgagacgctgaagctgttcaagcgtggcgcctacatcgtcaacaccgcccgcggcaagctgtgcgaccgcgatgccgtggcacgtgcgctcgaatccggccggctggccggctatgccggcgacgtgtggttcccgcagccggcgccgaaggaccacccctggcggacgatgccctataacggcatgaccccgcacatctccggcaccacgctgaccgcgcaggcgcgttatgcggcgggcacccgcgagatcctggagtgcttcttcgagggccgtccgatccgcgacgaatacctcatcgtgcagggcggcgctcttgccggcaccggcgcgcattcctactcgaagggcaatgccaccggcggttcggaagaggccgccaagttcaagaaggcggctgagaattcgtgaaaggagatatacatgaacctgagggagaagtacggtgagtggggcctgatcctgggcgcgaccgagggcgtcggcaaggcgttctgcgagaagatcgccgccggcggcatgaacgtcgtcatggtcggccgtcgcgaggagaagctgaacgtgctcgcaggcgagatccgcgagacctacggcgtggagaccaaggtcgtgcgcgccgactttagccagcccggcgctgccgagaccgtcttcgccgcgaccgagggcctggacatgggcttcatgagctacgtggcctgcctgcacagcttcggtaagatccaggacaccccctgggagaagcacgaggccatgatcaacgtcaacgtcgtgaccttcctcaagtgcttccaccactacatgcggatctttgccgcccaggaccgcggcgccgtgatcaacgtctcgtcgatgaccggcatcagctccagcccctggaacggccagtacggcgcgggcaaggccttcatcctcaagatgaccgaggccgtggcctgcgagtgcgagggcaccggcgtcgacgtcgaggtcatcaccctcggcaccaccctaacccccagcctgctgtccaacctccccggcggcccgcagggcgaggccgtcatgaagatcgccctcacccccgaggagtgcgttgacgaggcctttgagaagctgggtaaggagctctccgtcatcgccggccagcgcaacaaggactccgtccacgactggaaggcaaaccacaccgaggacgagtacatccgctacatggggtcgttctaccgcgactagaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 19 - protein, 404 aa, synthetic construct
  REGION 1..404: note = Description of Artificial Sequence: Synthetic construct polypeptide
MAKVLCVLYDDPVDGYPKTYARDDLPKIDHYPGGQILPTPKAIDFTPGQLLGSVSGELGLREYLESNGHTLVVTSDKDGPDSVFERELVDADVVISQPFWPAYLTPERIAKAKNLKLALTAGIGSDHVDLQSAIDRNVTVAEVTYCNSISVAEHVVMMILSLVRNYLPSHEWARKGGWNIADCVSHAYDLEAMHVGTVAAGRIGLAVLRRLAPFDVHLHYTGRHRLPESVEKELNLTWHATREDMYPVCDVVTLNCPLHPETEHMINDETLKLFKRGAYIVNTARGKLCDRDAVARALESGRLAGYAGDVWFPQPAPKDHPWRTMPYNGMTPHISGTTLTAQARYAAGTREILECFFEGRPIRDEYLIVQGGALAGTGAHSYSKGNATGGSEEAAKFKKAAENS

SEQ ID NO: 20 - protein, 263 aa, synthetic construct; identical to SEQ ID NO: 2
  REGION 1..263: note = Description of Artificial Sequence: Synthetic construct polypeptide

SEQ ID NO: 21 - DNA, 4302 bp, synthetic construct
  misc_feature 1..4302: note = Description of Artificial Sequence: Synthetic plasmid pCOLA(mod) 3alpha-HSDH polynucleotide
  source 1..4302: mol_type = other DNA
  CDS 303..1076: note = 3alpha-HSDH
ggggaattgtgagcggataacaattcccctgtagaaataattttgtttaactttaataaggagatataccatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgatgtccatcatcgtgataagcggctgcgccaccggcattggtgccgctacgcgcaaggtcctggaggcggccggtcaccagatcgtaggcatcgatatacgcgatgcggaagtgattgccgatctctcgacggccgaaggtcgaaagcaggcgattgccgatgtactggcgaagtgcagcaagggcatggacggcctggtgctgtgcgccggcctgggaccgcagaccaaggtgcttggcaatgtggtttcggtcaattattttggcgcgaccgagctgatggatgcctttttgccagcgctgaaaaaaggccatcagcccgcagccgtcgtcatctcgtccgtggcttccgcgcatctggcttttgacaagaacccactggcgctggcactggaagccggcgaggaagccaaggcccgcgccattgtcgaacatgcgggagagcagggcggaaatctggcctatgcgggcagcaagaatgctttgacggtggctgtgcgcaaacgcgccgccgcctggggcgaggctggcgtgcgcctgaacaccatcgcccccggtgcaaccgagactcccttgctgcaggcgggcctgcaggacccgcgctatggcgaatccattgccaagttcgttcctcccatgggccgccgtgccgagccgtccgagatggcgtcggtcatcgcctttttgatgagcccggccgcaagctatgtgcatggcgcgcagatcgtcattgatggcggcattgatgcggtgatgcgcccgacacagttctgagaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttacgccccgccctgccactcatcgcagtactgttgtaattcattaagcattctgccgacatggaagccatcacagacggcatgatgaacctgaatcgccagcggcatcagcaccttgtcgccttgcgtataatatttgcctatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtatttggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 22 - protein, 257 aa, synthetic construct [sequence omitted]

SEQ ID NO: 23 - DNA, 29 bp, synthetic construct
  misc_feature 1..29: note = Description of Artificial Sequence: Synthetic PCR primer
  source 1..29: mol_type = other DNA
cgatcatatggcaaaggtcctgtgcgttc

SEQ ID NO: 24 - DNA, 29 bp, synthetic construct
  misc_feature 1..29: note = Description of Artificial Sequence: Synthetic PCR primer
  source 1..29: mol_type = other DNA
gctagaattctcagccgccttcttgaact

SEQ ID NO: 25 - DNA, 768 bp, Escherichia coli
  source 1..768: mol_type = genomic DNA
  CDS 1..765
gtgtttaattctgacaacctgagactcgacggaaaatgcgccatcatcacaggtgcgggtgcaggtattggtaaagaaatcgccattacattcgcgacagctggcgcatctgtggtggtcagtgatattaacgccgacgcagctaaccatgttgtagacgaaattcaacaactgggtggtcaggcatttgcctgccgttgtgatattacttccgaacaggaactctctgcactggcagactttgctatcagtaagctgggtaaagttgatattctggttaacaacgccggtggcggtggacctaaaccgtttgatatgccaatggcggattttcgccgtgcttatgaactgaatgtgttttcttttttccatctgtcacaacttgttgcgccagaaatggaaaaaaatggcggtggcgttattctgaccatcacttctatggcggcagaaaataaaaatataaacatgacttcctatgcatcatctaaagctgcggccagtcatctggtcagaaatatggcgtttgacctaggtgaaaaaaatattcgggtaaatggcattgcgccgggggcaatattaaccgatgccctgaaatccgttattacaccagaaattgaacaaaaaatgttacagcacacgccgatcagacgtctgggccaaccgcaagatattgctaacgcagcgctgttcctttgctcgcctgctgcgagctgggtaagcggacaaattctcaccgtctccggtggtggggtacaggagctcaattaa

SEQ ID NO: 26 - protein, 255 aa, Escherichia coli
VFNSDNLRLDGKCAIITGAGAGIGKEIAITFATAGASVVVSDINADAANHVVDEIQQLGGQAFACRCDITSEQELSALADFAISKLGKVDILVNNAGGGGPKPFDMPMADFRRAYELNVFSFFHLSQLVAPEMEKNGGGVILTITSMAAENKNINMTSYASSKAAASHLVRNMAFDLGEKNIRVNGIAPGAILTDALKSVITPEIEQKMLQHTPIRRLGQPQDIANAALFLCSPAASWVSGQILTVSGGGVQELN

SEQ ID NO: 27 - DNA, 53 bp, synthetic construct
  misc_feature 1..53: note = Description of Artificial Sequence: Synthetic PCR primer
  source 1..53: mol_type = other DNA
aaaaaagcttataattatccttataggacgtcatggtgcgcccagatagggtg

SEQ ID NO: 28 - DNA, 60 bp, synthetic construct
  misc_feature 1..60: note = Description of Artificial Sequence: Synthetic PCR primer
  source 1..60: mol_type = other DNA
cagattgtacaaatgtggtgataacagataagtcgtcatgtttaacttacctttctttgt

SEQ ID NO: 29 - DNA, 49 bp, synthetic construct
  misc_feature 1..49: note = Description of Artificial Sequence: Synthetic PCR primer
  source 1..49: mol_type = other DNA
tgaacgcaagtttctaatttcggtttcctatcgatagaggaaagtgtct

SEQ ID NO: 30 - DNA, 29 bp, synthetic construct
  misc_feature 1..29: note = Description of Artificial Sequence: Synthetic PCR primer
  source 1..29: mol_type = other DNA
cctgcactacaccggccgtcaccgcctgc

SEQ ID NO: 31 - DNA, 23 bp, synthetic construct
  misc_feature 1..23: note = Description of Artificial Sequence: Synthetic PCR primer
  source 1..23: mol_type = other DNA
gctcgaattctcagaccgccttc

SEQ ID NO: 32 - DNA, 27 bp, synthetic construct
  misc_feature 1..27: note = Description of Artificial Sequence: Synthetic PCR primer
  source 1..27: mol_type = other DNA
ttaattgagctcctgtaccccaccacc

SEQ ID NO: 33 - DNA, 30 bp, synthetic construct
  misc_feature 1..30: note = Description of Artificial Sequence: Synthetic PCR primer
  source 1..30: mol_type = other DNA
gtgtttaattctgacaacctgagactcgac

SEQ ID NO: 34 - DNA, 1266 bp, synthetic construct
  misc_feature 1..1266: note = Description of Artificial Sequence: Synthetic FDH D221G mutant with His-tag polynucleotide
  source 1..1266: mol_type = other DNA
  CDS 1..1263
atggcaaaggtcctgtgcgttctttacgatgatccggtcgacggctacccgaagacctatgcccgcgacgatcttccgaagatcgaccactatccgggcggccagatcttgccgacgccgaaggccatcgacttcacgcccgggcagttgctcggctccgtctccggcgagctcggcctgcgcgaatatctcgaatccaacggccacaccctggtcgtgacctccgacaaggacggccccgactcggtgttcgagcgcgagctggtcgatgcggatgtcgtcatctcccagcccttctggccggcctatctgacgcccgagcgcatcgccaaggccaagaacctgaagctcgcgctcaccgccggcatcggttccgaccacgtcgatcttcagtcggctatcgaccgcaacgtcaccgtggcggaagtcacctactgcaactcgatcagcgtcgccgagcatgtggtgatgatgatcctgtcgctggtgcgcaactatctgccctcgcacgaatgggcgcggaagggcggctggaacatcgccgactgcgtctcccacgcctacgacctcgaggcgatgcatgtcggcaccgtggccgccggccgcatcggtctcgcggtgctgcgccgtctggcgccgttcgacgtgcacctgcactacaccggccgtcaccgcctgccggaatcggtcgagaaggagctcaacctcacctggcacgcgacccgcgaggacatgtatccggtttgcgacgtggtgacgctgaactgcccgctgcaccccgaaaccgagcacatgatcaatgacgagacgctgaagctgttcaagcgtggcgcctacatcgtcaacaccgcccgcggcaagctgtgcgaccgcgatgccgtggcacgtgcgctcgaatccggccggctggccggctatgccggcgacgtgtggttcccgcagccggcgccgaaggaccacccctggcggacgatgccctataacggcatgaccccgcacatctccggcaccacgctgaccgcgcaggcgcgttatgcggcgggcacccgcgagatcctggagtgcttcttcgagggccgtccgatccgcgacgaatacctcatcgtgcagggcggcgctcttgccggcaccggcgcgcattcctactcgaagggcaatgccaccggcggttcggaagaggccgccaagttcaagaaggcggctgagaattcgagctccgtcgacaagcttgcggccgcactcgagcaccaccaccaccaccactga

SEQ ID NO: 35 - protein, 421 aa, synthetic construct
  REGION 1..421: note = Description of Artificial Sequence: Synthetic construct polypeptide
MAKVLCVLYDDPVDGYPKTYARDDLPKIDHYPGGQILPTPKAIDFTPGQLLGSVSGELGLREYLESNGHTLVVTSDKDGPDSVFERELVDADVVISQPFWPAYLTPERIAKAKNLKLALTAGIGSDHVDLQSAIDRNVTVAEVTYCNSISVAEHVVMMILSLVRNYLPSHEWARKGGWNIADCVSHAYDLEAMHVGTVAAGRIGLAVLRRLAPFDVHLHYTGRHRLPESVEKELNLTWHATREDMYPVCDVVTLNCPLHPETEHMINDETLKLFKRGAYIVNTARGKLCDRDAVARALESGRLAGYAGDVWFPQPAPKDHPWRTMPYNGMTPHISGTTLTAQARYAAGTREILECFFEGRPIRDEYLIVQGGALAGTGAHSYSKGNATGGSEEAAKFKKAAENSSSVDKLAAALEHHHHHH

SEQ ID NO: 36 - protein, 401 aa, Mycobacterium vaccae
MAKVLCVLYDDPVDGYPKTYARDDLPKIDHYPGGQILPTPKAIDFTPGQLLGSVSGELGLREYLESNGHTLVVTSDKDGPDSVFERELVDADVVISQPFWPAYLTPERIAKAKNLKLALTAGIGSDHVDLQSAIDRNVTVAEVTYCNSISVAEHVVMMILSLVRNYLPSHEWARKGGWNIADCVSHAYDLEAMHVGTVAAGRIGLAVLRRLAPFDVHLHYTDRHRLPESVEKELNLTWHATREDMYPVCDVVTLNCPLHPETEHMINDETLKLFKRGAYIVNTARGKLCDRDAVARALESGRLAGYAGDVWFPQPAPKDHPWRTMPYNGMTPHISGTTLTAQARYAAGTREILECFFEGRPIRDEYLIVQGGALAGTGAHSYSKGNATGGSEEAAKFKKAV

SEQ ID NO: 37 - protein, 263 aa, synthetic construct
  REGION 1..263: note = Description of Artificial Sequence: Synthetic polypeptide derived from Collinsella aerofaciens (G39D)
MNLREKYGEWGLILGATEGVGKAFCEKIAAGGMNVVMVDRREEKLNVLAGEIRETYGVETKVVRADFSQPGAAETVFAATEGLDMGFMSYVACLHSFGKIQDTPWEKHEAMINVNVVTFLKCFHHYMRIFAAQDRGAVINVSSMTGISSSPWNGQYGAGKAFILKMTEAVACECEGTGVDVEVITLGTTLTPSLLSNLPGGPQGEAVMKIALTPEECVDEAFEKLGKELSVIAGQRNKDSVHDWKANHTEDEYIRYMGSFYRD

SEQ ID NO: 38 - protein, 263 aa, synthetic construct
  REGION 1..263: note = Description of Artificial Sequence: Synthetic polypeptide derived from Collinsella aerofaciens (G39D R40L)
MNLREKYGEWGLILGATEGVGKAFCEKIAAGGMNVVMVDLREEKLNVLAGEIRETYGVETKVVRADFSQPGAAETVFAATEGLDMGFMSYVACLHSFGKIQDTPWEKHEAMINVNVVTFLKCFHHYMRIFAAQDRGAVINVSSMTGISSSPWNGQYGAGKAFILKMTEAVACECEGTGVDVEVITLGTTLTPSLLSNLPGGPQGEAVMKIALTPEECVDEAFEKLGKELSVIAGQRNKDSVHDWKANHTEDEYIRYMGSFYRD

SEQ ID NO: 39 - protein, 263 aa, synthetic construct
  REGION 1..263: note = Description of Artificial Sequence: Synthetic polypeptide derived from Collinsella aerofaciens (G39D R40I)
MNLREKYGEWGLILGATEGVGKAFCEKIAAGGMNVVMVDIREEKLNVLAGEIRETYGVETKVVRADFSQPGAAETVFAATEGLDMGFMSYVACLHSFGKIQDTPWEKHEAMINVNVVTFLKCFHHYMRIFAAQDRGAVINVSSMTGISSSPWNGQYGAGKAFILKMTEAVACECEGTGVDVEVITLGTTLTPSLLSNLPGGPQGEAVMKIALTPEECVDEAFEKLGKELSVIAGQRNKDSVHDWKANHTEDEYIRYMGSFYRD

SEQ ID NO: 40 - protein, 263 aa, synthetic construct
  REGION 1..263: note = Description of Artificial Sequence: Synthetic polypeptide derived from Collinsella aerofaciens
MNLREKYGEWGLILGATEGVGKAFCEKIAAGGMNVVMVDVREEKLNVLAGEIRETYGVETKVVRADFSQPGAAETVFAATEGLDMGFMSYVACLHSFGKIQDTPWEKHEAMINVNVVTFLKCFHHYMRIFAAQDRGAVINVSSMTGISSSPWNGQYGAGKAFILKMTEAVACECEGTGVDVEVITLGTTLTPSLLSNLPGGPQGEAVMKIALTPEECVDEAFEKLGKELSVIAGQRNKDSVHDWKANHTEDEYIRYMGSFYRD

SEQ ID NO: 41 - DNA, 26 bp, synthetic construct
  misc_feature 1..26: note = Description of Artificial Sequence: Synthetic PCR primer
  source 1..26: mol_type = other DNA
cgtcgtcatggtcgaccgtcgcgagg

SEQ ID NO: 42 - DNA, 26 bp, synthetic construct
  misc_feature 1..26: note = Description of Artificial Sequence: Synthetic PCR primer
  source 1..26: mol_type = other DNA
cctcgcgacggtcgaccatgacgacg

SEQ ID NO: 43 - DNA, 26 bp, synthetic construct
  misc_feature 1..26: note = Description of Artificial Sequence: Synthetic PCR primer
  source 1..26: mol_type = other DNA
cgtcgtcatggtcgacctgcgcgagg

SEQ ID NO: 44 - DNA, 31 bp, synthetic construct
  misc_feature 1..31: note = Description of Artificial Sequence: Synthetic PCR primer
  source 1..31: mol_type = other DNA
ccgccgcatccataccgccagttgtttaccc

SEQ ID NO: 45 - DNA, 26 bp, synthetic construct
  misc_feature 1..26: note = Description of Artificial Sequence: Synthetic PCR primer
  source 1..26: mol_type = other DNA
cgtcgtcatggtcgacattcgcgagg

SEQ ID NO: 46 - DNA, 26 bp, synthetic construct
  misc_feature 1..26: note = Description of Artificial Sequence: Synthetic PCR primer
  source 1..26: mol_type = other DNA
cgtcgtcatggtcgacgttcgcgagg

SEQ ID NO: 47 - DNA, 786 bp, Bacillus subtilis
  source 1..786: mol_type = genomic DNA
atgtatccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatatcaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggttaa

SEQ ID NO: 48 - protein, 261 aa, Bacillus subtilis
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLENPVPSHEMPLKDWDKVIGTNLTGAFLGSREAIKYFVENDIKGNVINMSSVHEVIPWPLFVHYAASKGGIKLMTETLALEYAPKGIRVNNIGPGAINTPINAEKFADPKQKADVESMIPMGYIGEPEEIAAVAAWLASKEASYVTGITLFADGGMTQYPSFQAGRG

SEQ ID NO: 49 - DNA, 7404 bp, synthetic construct
  misc_feature 1..7404: note = Description of Artificial Sequence: Synthetic vector pFr7(D) polynucleotide
  source 1..7404: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttctagtcgcggtagaacgaccccatgtagcggatgtactcgtcctcggtgtggtttgccttccagtcgtggacggagtccttgttgcgctggccggcgatgacggagagctccttacccagcttctcaaaggcctcgtcaacgcactcctcgggggtgagggcgatcttcatgacggcctcgccctgcgggccgccggggaggttggacagcaggctgggggttagggtggtgccgagggtgatgacctcgacgtcgacgccggtgccctcgcactcgcaggccacggcctcggtcatcttgaggatgaaggccttgcccgcgccgtactggccgttccaggggctggagctgatgccggtcatcgacgagacgttgatcacggcgccgcggtcctgggcggcaaagatccgcatgtagtggtggaagcacttgaggaaggtcacgacgttgacgttgatcatggcctcgtgcttctcccagggggtgtcctggatcttaccgaagctgtgcaggcaggccacgtagctcatgaagcccatgtccaggccctcggtcgcggcgaagacggtctcggcagcgccgggctggctaaagtcggcgcgcacgaccttggtctccacgccgtaggtctcgcggatctcgcctgcgagcacgttcagcttctcctcgcgacggtcgaccatgacgacgttcatgccgccggcggcgatcttctcgcagaacgccttgccgacgccctcggtcgcgcccaggatcaggccccactcaccgtacttctccctcaggttcatgtatatctcctttcacgaattctcagccgccttcttgaacttggcggcctcttccgaaccgccggtggcattgcccttcgagtaggaatgcgcgccggtgccggcaagagcgccgccctgcacgatgaggtattcgtcgcggatcggacggccctcgaagaagcactccaggatctcgcgggtgcccgccgcataacgcgcctgcgcggtcagcgtggtgccggagatgtgcggggtcatgccgttatagggcatcgtccgccaggggtggtccttcggcgccggctgcgggaaccacacgtcgccggcatagccggccagccggccggattcgagcgcacgtgccacggcatcgcggtcgcacagcttgccgcgggcggtgttgacgatgtaggcgccacgcttgaacagcttcagcgtctcgtcattgatcatgtgctcggtttcggggtgcagcgggcagttcagcgtcaccacgtcgcaaaccggatacatgtcctcgcgggtcgcgtgccaggtgaggttgagctccttctcgaccgattccggcaggcggtgacggccggtgtagtgcaggtgcacgtcgaacggcgccagacggcgcagcaccgcgagaccgatgcggccggcggccacggtgccgacatgcatcgcctcgaggtcgtaggcgtgggagacgcagtcggcgatgttccagccgcccttccgcgcccattcgtgcgagggcagatagttgcgcaccagcgacaggatcatcatcaccacatgctcggcgacgctgatcgagttgcagtaggtgacttccgccacggtgacgttgcggtcgatagccgactgaagatcgacgtggtcggaaccgatgccggcggtgagcgcgagcttcaggttcttggccttggcgatgcgctcgggcgtcagataggccggccagaagggctgggagatgacgacatccgcatcgaccagctcgcgctcgaacaccgagtcggggccgtccttgtcggaggtcacgaccagggtgtggccgttggattcgagatattcgcgcaggccgagctcgccggagacggagccgagcaactgcccgggcgtgaagtcgatggccttcggcgtcggcaagatctggccgcccggatagtggtcgatcttcggaagatcgtcgcgggcataggtcttcgggtagccgtcgaccggatcatcgtaaagaacgcacaggacctttgccatcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 50 - DNA, 7404 bp, synthetic construct
  misc_feature 1..7404: note = Description of Artificial Sequence: Synthetic vector pFr7(DL) polynucleotide
  source 1..7404: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttctagtcgcggtagaacgaccccatgtagcggatgtactcgtcctcggtgtggtttgccttccagtcgtggacggagtccttgttgcgctggccggcgatgacggagagctccttacccagcttctcaaaggcctcgtcaacgcactcctcgggggtgagggcgatcttcatgacggcctcgccctgcgggccgccggggaggttggacagcaggctgggggttagggtggtgccgagggtgatgacctcgacgtcgacgccggtgccctcgcactcgcaggccacggcctcggtcatcttgaggatgaaggccttgcccgcgccgtactggccgttccaggggctggagctgatgccggtcatcgacgagacgttgatcacggcgccgcggtcctgggcggcaaagatccgcatgtagtggtggaagcacttgaggaaggtcacgacgttgacgttgatcatggcctcgtgcttctcccagggggtgtcctggatcttaccgaagctgtgcaggcaggccacgtagctcatgaagcccatgtccaggccctcggtcgcggcgaagacggtctcggcagcgccgggctggctaaagtcggcgcgcacgaccttggtctccacgccgtaggtctcgcggatctcgcctgcgagcacgttcagcttctcctcgcgcaggtcgaccatgacgacgttcatgccgccggcggcgatcttctcgcagaacgccttgccgacgccctcggtcgcgcccaggatcaggccccactcaccgtacttctccctcaggttcatgtatatctcctttcacgaattctcagccgccttcttgaacttggcggcctcttccgaaccgccggtggcattgcccttcgagtaggaatgcgcgccggtgccggcaagagcgccgccctgcacgatgaggtattcgtcgcggatcggacggccctcgaagaagcactccaggatctcgcgggtgcccgccgcataacgcgcctgcgcggtcagcgtggtgccggagatgtgcggggtcatgccgttatagggcatcgtccgccaggggtggtccttcggcgccggctgcgggaaccacacgtcgccggcatagccggccagccggccggattcgagcgcacgtgccacggcatcgcggtcgcacagcttgccgcgggcggtgttgacgatgtaggcgccacgcttgaacagcttcagcgtctcgtcattgatcatgtgctcggtttcggggtgcagcgggcagttcagcgtcaccacgtcgcaaaccggatacatgtcctcgcgggtcgcgtgccaggtgaggttgagctccttctcgaccgattccggcaggcggtgacggccggtgtagtgcaggtgcacgtcgaacggcgccagacggcgcagcaccgcgagaccgatgcggccggcggccacggtgccgacatgcatcgcctcgaggtcgtaggcgtgggagacgcagtcggcgatgttccagccgcccttccgcgcccattcgtgcgagggcagatagttgcgcaccagcgacaggatcatcatcaccacatgctcggcgacgctgatcgagttgcagtaggtgacttccgccacggtgacgttgcggtcgatagccgactgaagatcgacgtggtcggaaccgatgccggcggtgagcgcgagcttcaggttcttggccttggcgatgcgctcgggcgtcagataggccggccagaagggctgggagatgacgacatccgcatcgaccagctcgcgctcgaacaccgagtcggggccgtccttgtcggaggtcacgaccagggtgtggccgttggattcgagatattcgcgcaggccgagctcgccggagacggagccgagcaactgcccgggcgtgaagtcgatggccttcggcgtcggcaagatctggccgcccggatagtggtcgatcttcggaagatcgtcgcgggcataggtcttcgggtagccgtcgaccggatcatcgtaaagaacgcacaggacctttgccatcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 51 - DNA, 7404 bp, synthetic construct
  misc_feature 1..7404: note = Description of Artificial Sequence: Synthetic vector pFr7(DI) polynucleotide
  source 1..7404: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttctagtcgcggtagaacgaccccatgtagcggatgtactcgtcctcggtgtggtttgccttccagtcgtggacggagtccttgttgcgctggccggcgatgacggagagctccttacccagcttctcaaaggcctcgtcaacgcactcctcgggggtgagggcgatcttcatgacggcctcgccctgcgggccgccggggaggttggacagcaggctgggggttagggtggtgccgagggtgatgacctcgacgtcgacgccggtgccctcgcactcgcaggccacggcctcggtcatcttgaggatgaaggccttgcccgcgccgtactggccgttccaggggctggagctgatgccggtcatcgacgagacgttgatcacggcgccgcggtcctgggcggcaaagatccgcatgtagtggtggaagcacttgaggaaggtcacgacgttgacgttgatcatggcctcgtgcttctcccagggggtgtcctggatcttaccgaagctgtgcaggcaggccacgtagctcatgaagcccatgtccaggccctcggtcgcggcgaagacggtctcggcagcgccgggctggctaaagtcggcgcgcacgaccttggtctccacgccgtaggtctcgcggatctcgcctgcgagcacgttcagcttctcctcgcgaatgtcgaccatgacgacgttcatgccgccggcggcgatcttctcgcagaacgccttgccgacgccctcggtcgcgcccaggatcaggccccactcaccgtacttctccctcaggttcatgtatatctcctttcacgaattctcagccgccttcttgaacttggcggcctcttccgaaccgccggtggcattgcccttcgagtaggaatgcgcgccggtgccggcaagagcgccgccctgcacgatgaggtattcgtcgcggatcggacggccctcgaagaagcactccaggatctcgcgggtgcccgccgcataacgcgcctgcgcggtcagcgtggtgccggagatgtgcggggtcatgccgttatagggcatcgtccgccaggggtggtccttcggcgccggctgcgggaaccacacgtcgccggcatagccggccagccggccggattcgagcgcacgtgccacggcatcgcggtcgcacagcttgccgcgggcggtgttgacgatgtaggcgccacgcttgaacagcttcagcgtctcgtcattgatcatgtgctcggtttcggggtgcagcgggcagttcagcgtcaccacgtcgcaaaccggatacatgtcctcgcgggtcgcgtgccaggtgaggttgagctccttctcgaccgattccggcaggcggtgacggccggtgtagtgcaggtgcacgtcgaacggcgccagacggcgcagcaccgcgagaccgatgcggccggcggccacggtgccgacatgcatcgcctcgaggtcgtaggcgtgggagacgcagtcggcgatgttccagccgcccttccgcgcccattcgtgcgagggcagatagttgcgcaccagcgacaggatcatcatcaccacatgctcggcgacgctgatcgagttgcagtaggtgacttccgccacggtgacgttgcggtcgatagccgactgaagatcgacgtggtcggaaccgatgccggcggtgagcgcgagcttcaggttcttggccttggcgatgcgctcgggcgtcagataggccggccagaagggctgggagatgacgacatccgcatcgaccagctcgcgctcgaacaccgagtcggggccgtccttgtcggaggtcacgaccagggtgtggccgttggattcgagatattcgcgcaggccgagctcgccggagacggagccgagcaactgcccgggcgtgaagtcgatggccttcggcgtcggcaagatctggccgcccggatagtggtcgatcttcggaagatcgtcgcgggcataggtcttcgggtagccgtcgaccggatcatcgtaaagaacgcacaggacctttgccatcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 52 - DNA, 7404 bp, synthetic construct
  misc_feature 1..7404: note = Description of Artificial Sequence: Synthetic vector pFr7(DV) polynucleotide
  source 1..7404: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttctagtcgcggtagaacgaccccatgtagcggatgtactcgtcctcggtgtggtttgccttccagtcgtggacggagtccttgttgcgctggccggcgatgacggagagctccttacccagcttctcaaaggcctcgtcaacgcactcctcgggggtgagggcgatcttcatgacggcctcgccctgcgggccgccggggaggttggacagcaggctgggggttagggtggtgccgagggtgatgacctcgacgtcgacgccggtgccctcgcactcgcaggccacggcctcggtcatcttgaggatgaaggccttgcccgcgccgtactggccgttccaggggctggagctgatgccggtcatcgacgagacgttgatcacggcgccgcggtcctgggcggcaaagatccgcatgtagtggtggaagcacttgaggaaggtcacgacgttgacgttgatcatggcctcgtgcttctcccagggggtgtcctggatcttaccgaagctgtgcaggcaggccacgtagctcatgaagcccatgtccaggccctcggtcgcggcgaagacggtctcggcagcgccgggctggctaaagtcggcgcgcacgaccttggtctccacgccgtaggtctcgcggatctcgcctgcgagcacgttcagcttctcctcgcgaacgtcgaccatgacgacgttcatgccgccggcggcgatcttctcgcagaacgccttgccgacgccctcggtcgcgcccaggatcaggccccactcaccgtacttctccctcaggttcatgtatatctcctttcacgaattctcagccgccttcttgaacttggcggcctcttccgaaccgccggtggcattgcccttcgagtaggaatgcgcgccggtgccggcaagagcgccgccctgcacgatgaggtattcgtcgcggatcggacggccctcgaagaagcactccaggatctcgcgggtgcccgccgcataacgcgcctgcgcggtcagcgtggtgccggagatgtgcggggtcatgccgttatagggcatcgtccgccaggggtggtccttcggcgccggctgcgggaaccacacgtcgccggcatagccggccagccggccggattcgagcgcacgtgccacggcatcgcggtcgcacagcttgccgcgggcggtgttgacgatgtaggcgccacgcttgaacagcttcagcgtctcgtcattgatcatgtgctcggtttcggggtgcagcgggcagttcagcgtcaccacgtcgcaaaccggatacatgtcctcgcgggtcgcgtgccaggtgaggttgagctccttctcgaccgattccggcaggcggtgacggccggtgtagtgcaggtgcacgtcgaacggcgccagacggcgcagcaccgcgagaccgatgcggccggcggccacggtgccgacatgcatcgcctcgaggtcgtaggcgtgggagacgcagtcggcgatgttccagccgcccttccgcgcccattcgtgcgagggcagatagttgcgcaccagcgacaggatcatcatcaccacatgctcggcgacgctgatcgagttgcagtaggtgacttccgccacggtgacgttgcggtcgatagccgactgaagatcgacgtggtcggaaccgatgccggcggtgagcgcgagcttcaggttcttggccttggcgatgcgctcgggcgtcagataggccggccagaagggctgggagatgacgacatccgcatcgaccagctcgcgctcgaacaccgagtcggggccgtccttgtcggaggtcacgaccagggtgtggccgttggattcgagatattcgcgcaggccgagctcgccggagacggagccgagcaactgcccgggcgtgaagtcgatggccttcggcgtcggcaagatctggccgcccggatagtggtcgatcttcggaagatcgtcgcgggcataggtcttcgggtagccgtcgaccggatcatcgtaaagaacgcacaggacctttgccatcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 53 - DNA, 27 bp, synthetic construct
  misc_feature 1..27: note = Description of Artificial Sequence: Synthetic PCR primer "G39D for"
  source 1..27: mol_type = other DNA
gtcgtcatggtcgaccgtcgcgaggag

SEQ ID NO: 54 - DNA, 27 bp, synthetic construct
  misc_feature 1..27: note = Description of Artificial Sequence: Synthetic PCR primer "G39D_rev"
  source 1..27: mol_type = other DNA
ctcctcgcgacggtcgaccatgacgac

SEQ ID NO: 55 - DNA, 24 bp, synthetic construct
  misc_feature 1..24: note = Description of Artificial Sequence: Synthetic PCR primer "G39D/R40I_for"
  source 1..24: mol_type = other DNA
gtcatggtcgacattcgcgaggag

SEQ ID NO: 56 - DNA, 24 bp, synthetic construct
  misc_feature 1..24: note = Description of Artificial Sequence: Synthetic PCR primer "G39D/R40I_rev"
  source 1..24: mol_type = other DNA
ctcctcgcgaatgtcgaccatgac

SEQ ID NO: 57 - DNA, 27 bp, synthetic construct
  misc_feature 1..27: note = Description of Artificial Sequence: Synthetic PCR primer "R40D_for"
  source 1..27: mol_type = other DNA
gtcatggtcggcgatcgcgaggagaag

SEQ ID NO: 58 - DNA, 27 bp, synthetic construct
  misc_feature 1..27: note = Description of Artificial Sequence: Synthetic PCR primer "R40D_rev"
  source 1..27: mol_type = other DNA
cttctcctcgcgatcgccgaccatgac

SEQ ID NO: 59 - DNA, 30 bp, synthetic construct
  misc_feature 1..30: note = Description of Artificial Sequence: Synthetic PCR primer "R40D/R41I_for"
  source 1..30: mol_type = other DNA
gtcatggtcggcgatatcgaggagaagctg

SEQ ID NO: 60 - DNA, 30 bp, synthetic construct
  misc_feature 1..30: note = Description of Artificial Sequence: Synthetic PCR primer "R40D/R41I_rev"
  source 1..30: mol_type = other DNA
cagcttctcctcgatatcgccgaccatgac

SEQ ID NO: 61 - DNA, 27 bp, synthetic construct
  misc_feature 1..27: note = Description of Artificial Sequence: Synthetic PCR primer "DIN_for"
  source 1..27: mol_type = other DNA
atggtcgacattaacgaggagaagctg

SEQ ID NO: 62 - DNA, 27 bp, synthetic construct
  misc_feature 1..27: note = Description of Artificial Sequence: Synthetic PCR primer "DIN_rev"
  source 1..27: mol_type = other DNA
cagcttctcctcgttaatgtcgaccat

SEQ ID NO: 63 - DNA, 8321 bp, synthetic construct
  misc_feature 1..8321: note = Description of Artificial Sequence: Synthetic plasmid pFr3T7(D) polynucleotide
  source 1..8321: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcctagtcgcggtagaacgaccccatgtagcggatgtactcgtcctcggtgtggtttgccttccagtcgtggacggagtccttgttgcgctggccggcgatgacggagagctccttacccagcttctcaaaggcctcgtcaacgcactcctcgggggtgagggcgatcttcatgacggcctcgccctgcgggccgccggggaggttggacagcaggctgggggttagggtggtgccgagggtgatgacctcgacgtcgacgccggtgccctcgcactcgcaggccacggcctcggtcatcttgaggatgaaggccttgcccgcgccgtactggccgttccaggggctggagctgatgccggtcatcgacgagacgttgatcacggcgccgcggtcctgggcggcaaagatccgcatgtagtggtggaagcacttgaggaaggtcacgacgttgacgttgatcatggcctcgtgcttctcccagggggtgtcctggatcttaccgaagctgtgcaggcaggccacgtagctcatgaagcccatgtccaggccctcggtcgcggcgaagacggtctcggcagcgccgggctggctaaagtcggcgcgcacgaccttggtctccacgccgtaggtctcgcggatctcgcctgcgagcacgttcagcttctcctcgcgacggtcgaccatgacgacgttcatgccgccggcggcgatcttctcgcagaacgccttgccgacgccctcggtcgcgcccaggatcaggccccactcaccgtacttctccctcaggttcatatgtatatctccttcttatacttaactaatatactaagatggggaattgttatccgctcacaattcccctatagtgagtcgtattaatttcgattatgcggccgtgtacaatacgattactttctgttcgacttaagcattataagctttcagaactgtgtcgggcgcatcaccgcatcaatgccgccatcaatgacgatctgcgcgccatgcacatagcttgcggccgggctcatcaaaaaggcgatgaccgacgccatctcggacggctcggcacggcggcccatgggaggaacgaacttggcaatggattcgccatagcgcgggtcctgcaggcccgcctgcagcaagggagtctcggttgcaccgggggcgatggtgttcaggcgcacgccagcctcgccccaggcggcggcgcgtttgcgcacagccaccgtcaaagcattcttgctgcccgcataggccagatttccgccctgctctcccgcatgttcgacaatggcgcgggccttggcttcctcgccggcttccagtgccagcgccagtgggttcttgtcaaaagccagatgcgcggaagccacggacgagatgacgacggctgcgggctgatggccttttttcagcgctggcaaaaaggcatccatcagctcggtcgcgccaaaataattgaccgaaaccacattgccaagcaccttggtctgcggtcccaggccggcgcacagcaccaggccgtccatgcccttgctgcacttcgccagtacatcggcaatcgcctgctttcgaccttcggccgtcgagagatcggcaatcacttccgcatcgcgtatatcgatgcctacgatctggtgaccggccgcctccaggaccttgcgcgtagccgcaccaatgccggtggcgcagccgcttatcacgatgatggacatgtatatctcctttcacgaattctcagccgccttcttgaacttggcggcctcttccgaaccgccggtggcattgcccttcgagtaggaatgcgcgccggtgccggcaagagcgccgccctgcacgatgaggtattcgtcgcggatcggacggccctcgaagaagcactccaggatctcgcgggtgcccgccgcataacgcgcctgcgcggtcagcgtggtgccggagatgtgcggggtcatgccgttatagggcatcgtccgccaggggtggtccttcggcgccggctgcgggaaccacacgtcgccggcatagccggccagccggccggattcgagcgcacgtgccacggcatcgcggtcgcacagcttgccgcgggcggtgttgacgatgtaggcgccacgcttgaacagcttcagcgtctcgtcattgatcatgtgctcggtttcggggtgcagcgggcagttcagcgtcaccacgtcgcaaaccggatacatgtcctcgcgggtcgcgtgccaggtgaggttgagctccttctcgaccgattccggcaggcggtgacggtcggtgtagtgcaggtgcacgtcgaacggcgccagacggcgcagcaccgcgagaccgatgcggccggcggccacggtgccgacatgcatcgcctcgaggtcgtaggcgtgggagacgcagtcggcgatgttccagccgcccttccgcgcccattcgtgcgagggcagatagttgcgcaccagcgacaggatcatcatcaccacatgctcggcgacgctgatcgagttgcagtaggtgacttccgccacggtgacgttgcggtcgatagccgactgaagatcgacgtggtcggaaccgatgccggcggtgagcgcgagcttcaggttcttggccttggcgatgcgctcgggcgtcagataggccggccagaagggctgggagatgacgacatccgcatcgaccagctcgcgctcgaacaccgagtcggggccgtccttgtcggaggtcacgaccagggtgtggccgttggattcgagatattcgcgcaggccgagctcgccggagacggagccgagcaactgcccgggcgtgaagtcgatggccttcggcgtcggcaagatctggccgcccggatagtggtcgatcttcggaagatcgtcgcgggcataggtcttcgggtagccgtcgaccggatcatcgtaaagaacgcacaggacctttgccatcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 64 - DNA, 8190 bp, synthetic construct
  misc_feature 1..8190: note = Description of Artificial Sequence: Synthetic pF(G)r7(A)r3 polynucleotide
  source 1..8190: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgctcagaactgtgtcgggcgcatcaccgcatcaatgccgccatcaatgacgatctgcgcgccatgcacatagcttgcggccgggctcatcaaaaaggcgatgaccgacgccatctcggacggctcggcacggcggcccatgggaggaacgaacttggcaatggattcgccatagcgcgggtcctgcaggcccgcctgcagcaagggagtctcggttgcaccgggggcgatggtgttcaggcgcacgccagcctcgccccaggcggcggcgcgtttgcgcacagccaccgtcaaagcattcttgctgcccgcataggccagatttccgccctgctctcccgcatgttcgacaatggcgcgggccttggcttcctcgccggcttccagtgccagcgccagtgggttcttgtcaaaagccagatgcgcggaagccacggacgagatgacgacggctgcgggctgatggccttttttcagcgctggcaaaaaggcatccatcagctcggtcgcgccaaaataattgaccgaaaccacattgccaagcaccttggtctgcggtcccaggccggcgcacagcaccaggccgtccatgcccttgctgcacttcgccagtacatcggcaatcgcctgctttcgaccttcggccgtcgagagatcggcaatcacttccgcatcgcgtatatcgatgcctacgatctggtgaccggccgcctccaggaccttgcgcgtagcggcaccaatgccggtggcgcagccgcttatcacgatgatggacatgtatatctccttaagcttctagtcgcggtagaacgaccccatgtagcggatgtactcgtcctcggtgtggtttgccttccagtcgtggacggagtccttgttgcgctggccggcgatgacggagagctccttacccagcttctcaaaggcctcgtcaacgcactcctcgggggtgagggcgatcttcatgacggcctcgccctgcgggccgccggggaggttggacagcaggctgggggttagggtggtgccgagggtgatgacctcgacgtcgacgccggtgccctcgcactcgcaggccacggcctcggtcatcttgaggatgaaggccttgcccgcgccgtactggccgttccaggggctggagctgatgccggtcatcgacgagacgttgatcacggcgccgcggtcctgggcggcaaagatccgcatgtagtggtggaagcacttgaggaaggtcacgacgttgacgttgatcatggcctcgtgcttctcccagggggtgtcctggatcttaccgaagctgtgcaggcaggccacgtagctcatgaagcccatgtccaggccctcggtcgcggcgaagacggtctcggcagcgccgggctggctaaagtcggcgcgcacgaccttggtctccacgccgtaggtctcgcggatctcgcctgcgagcacgttcagcttctcctcgcgacgggcgaccatgacgacgttcatgccgccggcggcgatcttctcgcagaacgccttgccgacgccctcggtcgcgcccaggatcaggccccactcaccgtacttctccctcaggttcatgtatatctcctttcacgaattctcagccgccttcttgaacttggcggcctcttccgaaccgccggtggcattgcccttcgagtaggaatgcgcgccggtgccggcaagagcgccgccctgcacgatgaggtattcgtcgcggatcggacggccctcgaagaagcactccaggatctcgcgggtgcccgccgcataacgcgcctgcgcggtcagcgtggtgccggagatgtgcggggtcatgccgttatagggcatcgtccgccaggggtggtccttcggcgccggctgcgggaaccacacgtcgccggcatagccggccagccggccggattcgagcgcacgtgccacggcatcgcggtcgcacagcttgccgcgggcggtgttgacgatgtaggcgccacgcttgaacagcttcagcgtctcgtcattgatcatgtgctcggtttcggggtgcagcgggcagttcagcgtcaccacgtcgcaaaccggatacatgtcctcgcgggtcgcgtgccaggtgaggttgagctccttctcgaccgattccggcaggcggtgacggccggtgtagtgcaggtgcacgtcgaacggcgccagacggcgcagcaccgcgagaccgatgcggccggcggccacggtgccgacatgcatcgcctcgaggtcgtaggcgtgggagacgcagtcggcgatgttccagccgcccttccgcgcccattcgtgcgagggcagatagttgcgcaccagcgacaggatcatcatcaccacatgctcggcgacgctgatcgagttgcagtaggtgacttccgccacggtgacgttgcggtcgatagccgactgaagatcgacgtggtcggaaccgatgccggcggtgagcgcgagcttcaggttcttggccttggcgatgcgctcgggcgtcagataggccggccagaagggctgggagatgacgacatccgcatcgaccagctcgcgctcgaacaccgagtcggggccgtccttgtcggaggtcacgaccagggtgtggccgttggattcgagatattcgcgcaggccgagctcgccggagacggagccgagcaactgcccgggcgtgaagtcgatggccttcggcgtcggcaagatctggccgcccggatagtggtcgatcttcggaagatcgtcgcgggcataggtcttcgggtagccgtcgaccggatcatcgtaaagaacgcacaggacctttgccatcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 65 - DNA, 8190 bp, synthetic construct
  misc_feature 1..8190: note = Description of Artificial Sequence: Synthetic pF(G)r7(S)r3 polynucleotide
  source 1..8190: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgctcagaactgtgtcgggcgcatcaccgcatcaatgccgccatcaatgacgatctgcgcgccatgcacatagcttgcggccgggctcatcaaaaaggcgatgaccgacgccatctcggacggctcggcacggcggcccatgggaggaacgaacttggcaatggattcgccatagcgcgggtcctgcaggcccgcctgcagcaagggagtctcggttgcaccgggggcgatggtgttcaggcgcacgccagcctcgccccaggcggcggcgcgtttgcgcacagccaccgtcaaagcattcttgctgcccgcataggccagatttccgccctgctctcccgcatgttcgacaatggcgcgggccttggcttcctcgccggcttccagtgccagcgccagtgggttcttgtcaaaagccagatgcgcggaagccacggacgagatgacgacggctgcgggctgatggccttttttcagcgctggcaaaaaggcatccatcagctcggtcgcgccaaaataattgaccgaaaccacattgccaagcaccttggtctgcggtcccaggccggcgcacagcaccaggccgtccatgcccttgctgcacttcgccagtacatcggcaatcgcctgctttcgaccttcggccgtcgagagatcggcaatcacttccgcatcgcgtatatcgatgcctacgatctggtgaccggccgcctccaggaccttgcgcgtagcggcaccaatgccggtggcgcagccgcttatcacgatgatggacatgtatatctccttaagcttctagtcgcggtagaacgaccccatgtagcggatgtactcgtcctcggtgtggtttgccttccagtcgtggacggagtccttgttgcgctggccggcgatgacggagagctccttacccagcttctcaaaggcctcgtcaacgcactcctcgggggtgagggcgatcttcatgacggcctcgccctgcgggccgccggggaggttggacagcaggctgggggttagggtggtgccgagggtgatgacctcgacgtcgacgccggtgccctcgcactcgcaggccacggcctcggtcatcttgaggatgaaggccttgcccgcgccgtactggccgttccaggggctggagctgatgccggtcatcgacgagacgttgatcacggcgccgcggtcctgggcggcaaagatccgcatgtagtggtggaagcacttgaggaaggtcacgacgttgacgttgatcatggcctcgtgcttctcccagggggtgtcctggatcttaccgaagctgtgcaggcaggccacgtagctcatgaagcccatgtccaggccctcggtcgcggcgaagacggtctcggcagcgccgggctggctaaagtcggcgcgcacgaccttggtctccacgccgtaggtctcgcggatctcgcctgcgagcacgttcagcttctcctcgcgacggctgaccatgacgacgttcatgccgccggcggcgatcttctcgcagaacgccttgccgacgccctcggtcgcgcccaggatcaggccccactcaccgtacttctccctcaggttcatgtatatctcctttcacgaattctcagccgccttcttgaacttggcggcctcttccgaaccgccggtggcattgcccttcgagtaggaatgcgcgccggtgccggcaagagcgccgccctgcacgatgaggtattcgtcgcggatcggacggccctcgaagaagcactccaggatctcgcgggtgcccgccgcataacgcgcctgcgcggtcagcgtggtgccggagatgtgcggggtcatgccgttatagggcatcgtccgccaggggtggtccttcggcgccggctgcgggaaccacacgtcgccggcatagccggccagccggccggattcgagcgcacgtgccacggcatcgcggtcgcacagcttgccgcgggcggtgttgacgatgtaggcgccacgcttgaacagcttcagcgtctcgtcattgatcatgtgctcggtttcggggtgcagcgggcagttcagcgtcaccacgtcgcaaaccggatacatgtcctcgcgggtcgcgtgccaggtgaggttgagctccttctcgaccgattccggcaggcggtgacggccggtgtagtgcaggtgcacgtcgaacggcgccagacggcgcagcaccgcgagaccgatgcggccggcggccacggtgccgacatgcatcgcctcgaggtcgtaggcgtgggagacgcagtcggcgatgttccagccgcccttccgcgcccattcgtgcgagggcagatagttgcgcaccagcgacaggatcatcatcaccacatgctcggcgacgctgatcgagttgcagtaggtgacttccgccacggtgacgttgcggtcgatagccgactgaagatcgacgtggtcggaaccgatgccggcggtgagcgcgagcttcaggttcttggccttggcgatgcgctcgggcgtcagataggccggccagaagggctgggagatgacgacatccgcatcgaccagctcgcgctcgaacaccgagtcggggccgtccttgtcggaggtcacgaccagggtgtggccgttggattcgagatattcgcgcaggccgagctcgccggagacggagccgagcaactgcccgggcgtgaagtcgatggccttcggcgtcggcaagatctggccgcccggatagtggtcgatcttcggaagatcgtcgcgggcataggtcttcgggtagccgtcgaccggatcatcgtaaagaacgcacaggacctttgccatcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 66 - DNA, 7910 bp, synthetic construct
  misc_feature 1..7910: note = Description of Artificial Sequence: Synthetic p3T7(A)rG polynucleotide
  source 1..7910: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagttaaccgcggcctgcctggaatgaaggatattgtgtcataccgccgtccgcgaataacgtgatgcctgtgacgtagctggcttccttcgaagcaagccaggctgctactgcggcgatctcctccggttcgccgatatatcccattggaatcatgctttctacatcagctttctgtttagggtcagcgaatttttcagcattgattggcgtgttgatcgcacctggcccaatattattgacgcgaatgcccttcggcgcgtattccaacgctaatgtttctgtcatcagctttatcccgcctttacttgccgcatagtggacaaataacggccaaggaatcacttcgtgcacactggacatgttaatgacatttcccttgatatcgttttctacgaaatatttaatcgcttcacggcttcctaaaaaggcacccgttaagttcgtgccgatgactttatcccaatccttgagcggcatttcgtgagatggcacaggattttcaagaccggcattattaatcataatatcgagtgtgccgaactccttaattgccgtttgcacgatattttttacatcttcctctttcgtgacatctccttggacgacaacagcttcaccgcccgccttgatgacctcttcttttacctcgttcggatcttgtttattactataatagttgataaccacttttgcctgctccttgccgaagcgaatggccatcgcctttccgagccctgaagcagctcctgtaatagcgacgacttttccttttaaatccggatacatgtatatctccttgcggccgcctagtcgcggtagaacgaccccatgtagcggatgtactcgtcctcggtgtggtttgccttccagtcgtggacggagtccttgttgcgctggccggcgatgacggagagctccttacccagcttctcaaaggcctcgtcaacgcactcctcgggggtgagggcgatcttcatgacggcctcgccctgcgggccgccggggaggttggacagcaggctgggggttagggtggtgccgagggtgatgacctcgacgtcgacgccggtgccctcgcactcgcaggccacggcctcggtcatcttgaggatgaaggccttgcccgcgccgtactggccgttccaggggctggagctgatgccggtcatcgacgagacgttgatcacggcgccgcggtcctgggcggcaaagatccgcatgtagtggtggaagcacttgaggaaggtcacgacgttgacgttgatcatggcctcgtgcttctcccagggggtgtcctggatcttaccgaagctgtgcaggcaggccacgtagctcatgaagcccatgtccaggccctcggtcgcggcgaagacggtctcggcagcgccgggctggctaaagtcggcgcgcacgaccttggtctccacgccgtaggtctcgcggatctcgcctgcgagcacgttcagcttctcctcgcgacgggcgaccatgacgacgttcatgccgccggcggcgatcttctcgcagaacgccttgccgacgccctcggtcgcgcccaggatcaggccccactcaccgtacttctccctcaggttcatatgtatatctccttcttatacttaactaatatactaagatggggaattgttatccgctcacaattcccctatagtgagtcgtattaatttcgattatgcggccgtgtacaatacgattactttctgttcgacttaagcattataagcttgtcgacggagctcgaattctcagaactgtgtcgggcgcatcaccgcatcaatgccgccatcaatgacgatctgcgcgccatgcacatagcttgcggccgggctcatcaaaaaggcgatgaccgacgccatctcggacggctcggcacggcggcccatgggaggaacgaacttggcaatggattcgccatagcgcgggtcctgcaggcccgcctgcagcaagggagtctcggttgcaccgggggcgatggtgttcaggcgcacgccagcctcgccccaggcggcggcgcgtttgcgcacagccaccgtcaaagcattcttgctgcccgcataggccagatttccgccctgctctcccgcatgttcgacaatggcgcgggccttggcttcctcgccggcttccagtgccagcgccagtgggttcttgtcaaaagccagatgcgcggaagccacggacgagatgacgacggctgcgggctgatggccttttttcagcgctggcaaaaaggcatccatcagctcggtcgcgccaaaataattgaccgaaaccacattgccaagcaccttggtctgcggtcccaggccggcgcacagcaccaggccgtccatgcccttgctgcacttcgccagtacatcggcaatcgcctgctttcgaccttcggccgtcgagagatcggcaatcacttccgcatcgcgtatatcgatgcctacgatctggtgaccggccgcctccaggaccttgcgcgtagccgcaccaatgccggtggcgcagccgcttatcacgatgatggacatcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 67 - DNA, 7891 bp, synthetic construct
  misc_feature 1..7891: note = Description of Artificial Sequence: Synthetic p7(A)T3rG polynucleotide
  source 1..7891: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagttaaccgcggcctgcctggaatgaaggatattgtgtcataccgccgtccgcgaataacgtgatgcctgtgacgtagctggcttccttcgaagcaagccaggctgctactgcggcgatctcctccggttcgccgatatatcccattggaatcatgctttctacatcagctttctgtttagggtcagcgaatttttcagcattgattggcgtgttgatcgcacctggcccaatattattgacgcgaatgcccttcggcgcgtattccaacgctaatgtttctgtcatcagctttatcccgcctttacttgccgcatagtggacaaataacggccaaggaatcacttcgtgcacactggacatgttaatgacatttcccttgatatcgttttctacgaaatatttaatcgcttcacggcttcctaaaaaggcacccgttaagttcgtgccgatgactttatcccaatccttgagcggcatttcgtgagatggcacaggattttcaagaccggcattattaatcataatatcgagtgtgccgaactccttaattgccgtttgcacgatattttttacatcttcctctttcgtgacatctccttggacgacaacagcttcaccgcccgccttgatgacctcttcttttacctcgttcggatcttgtttattactataatagttgataaccacttttgcctgctccttgccgaagcgaatggccatcgcctttccgagccctgaagcagctcctgtaatagcgacgacttttccttttaaatccggatacatgtatatctccttgcggccgctcagaactgtgtcgggcgcatcaccgcatcaatgccgccatcaatgacgatctgcgcgccatgcacatagcttgcggccgggctcatcaaaaaggcgatgaccgacgccatctcggacggctcggcacggcggcccatgggaggaacgaacttggcaatggattcgccatagcgcgggtcctgcaggcccgcctgcagcaagggagtctcggttgcaccgggggcgatggtgttcaggcgcacgccagcctcgccccaggcggcggcgcgtttgcgcacagccaccgtcaaagcattcttgctgcccgcataggccagatttccgccctgctctcccgcatgttcgacaatggcgcgggccttggcttcctcgccggcttccagtgccagcgccagtgggttcttgtcaaaagccagatgcgcggaagccacggacgagatgacgacggctgcgggctgatggccttttttcagcgctggcaaaaaggcatccatcagctcggtcgcgccaaaataattgaccgaaaccacattgccaagcaccttggtctgcggtcccaggccggcgcacagcaccaggccgtccatgcccttgctgcacttcgccagtacatcggcaatcgcctgctttcgaccttcggccgtcgagagatcggcaatcacttccgcatcgcgtatatcgatgcctacgatctggtgaccggccgcctccaggaccttgcgcgtagcggcaccaatgccggtggcgcagccgcttatcacgatgatggacatcatatgtatatctccttcttatacttaactaatatactaagatggggaattgttatccgctcacaattcccctatagtgagtcgtattaatttcgattatgcggccgtgtacaatacgattactttctgttcgacttaagcattataagcttctagtcgcggtagaacgaccccatgtagcggatgtactcgtcctcggtgtggtttgccttccagtcgtggacggagtccttgttgcgctggccggcgatgacggagagctccttacccagcttctcaaaggcctcgtcaacgcactcctcgggggtgagggcgatcttcatgacggcctcgccctgcgggccgccggggaggttggacagcaggctgggggttagggtggtgccgagggtgatgacctcgacgtcgacgccggtgccctcgcactcgcaggccacggcctcggtcatcttgaggatgaaggccttgcccgcgccgtactggccgttccaggggctggagctgatgccggtcatcgacgagacgttgatcacggcgccgcggtcctgggcggcaaagatccgcatgtagtggtggaagcacttgaggaaggtcacgacgttgacgttgatcatggcctcgtgcttctcccagggggtgtcctggatcttaccgaagctgtgcaggcaggccacgtagctcatgaagcccatgtccaggccctcggtcgcggcgaagacggtctcggcagcgccgggctggctaaagtcggcgcgcacgaccttggtctccacgccgtaggtctcgcggatctcgcctgcgagcacgttcagcttctcctcgcgacgggcgaccatgacgacgttcatgccgccggcggcgatcttctcgcagaacgccttgccgacgccctcggtcgcgcccaggatcaggccccactcaccgtacttctccctcaggttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 68 - DNA, 7404 bp, synthetic construct
  misc_feature 1..7404: note = Description of Artificial Sequence: Synthetic pF(G)r7(A) polynucleotide
  source 1..7404: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttctagtcgcggtagaacgaccccatgtagcggatgtactcgtcctcggtgtggtttgccttccagtcgtggacggagtccttgttgcgctggccggcgatgacggagagctccttacccagcttctcaaaggcctcgtcaacgcactcctcgggggtgagggcgatcttcatgacggcctcgccctgcgggccgccggggaggttggacagcaggctgggggttagggtggtgccgagggtgatgacctcgacgtcgacgccggtgccctcgcactcgcaggccacggcctcggtcatcttgaggatgaaggccttgcccgcgccgtactggccgttccaggggctggagctgatgccggtcatcgacgagacgttgatcacggcgccgcggtcctgggcggcaaagatccgcatgtagtggtggaagcacttgaggaaggtcacgacgttgacgttgatcatggcctcgtgcttctcccagggggtgtcctggatcttaccgaagctgtgcaggcaggccacgtagctcatgaagcccatgtccaggccctcggtcgcggcgaagacggtctcggcagcgccgggctggctaaagtcggcgcgcacgaccttggtctccacgccgtaggtctcgcggatctcgcctgcgagcacgttcagcttctcctcgcgacgggcgaccatgacgacgttcatgccgccggcggcgatcttctcgcagaacgccttgccgacgccctcggtcgcgcccaggatcaggccccactcaccgtacttctccctcaggttcatgtatatctcctttcacgaattctcagccgccttcttgaacttggcggcctcttccgaaccgccggtggcattgcccttcgagtaggaatgcgcgccggtgccggcaagagcgccgccctgcacgatgaggtattcgtcgcggatcggacggccctcgaagaagcactccaggatctcgcgggtgcccgccgcataacgcgcctgcgcggtcagcgtggtgccggagatgtgcggggtcatgccgttatagggcatcgtccgccaggggtggtccttcggcgccggctgcgggaaccacacgtcgccggcatagccggccagccggccggattcgagcgcacgtgccacggcatcgcggtcgcacagcttgccgcgggcggtgttgacgatgtaggcgccacgcttgaacagcttcagcgtctcgtcattgatcatgtgctcggtttcggggtgcagcgggcagttcagcgtcaccacgtcgcaaaccggatacatgtcctcgcgggtcgcgtgccaggtgaggttgagctccttctcgaccgattccggcaggcggtgacggccggtgtagtgcaggtgcacgtcgaacggcgccagacggcgcagcaccgcgagaccgatgcggccggcggccacggtgccgacatgcatcgcctcgaggtcgtaggcgtgggagacgcagtcggcgatgttccagccgcccttccgcgcccattcgtgcgagggcagatagttgcgcaccagcgacaggatcatcatcaccacatgctcggcgacgctgatcgagttgcagtaggtgacttccgccacggtgacgttgcggtcgatagccgactgaagatcgacgtggtcggaaccgatgccggcggtgagcgcgagcttcaggttcttggccttggcgatgcgctcgggcgtcagataggccggccagaagggctgggagatgacgacatccgcatcgaccagctcgcgctcgaacaccgagtcggggccgtccttgtcggaggtcacgaccagggtgtggccgttggattcgagatattcgcgcaggccgagctcgccggagacggagccgagcaactgcccgggcgtgaagtcgatggccttcggcgtcggcaagatctggccgcccggatagtggtcgatcttcggaagatcgtcgcgggcataggtcttcgggtagccgtcgaccggatcatcgtaaagaacgcacaggacctttgccatcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 69 - DNA, 7386 bp, synthetic construct
  misc_feature 1..7386: note = Description of Artificial Sequence: Synthetic pFr3 polynucleotide
  source 1..7386: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagctttcagaactgtgtcgggcgcatcaccgcatcaatgccgccatcaatgacgatctgcgcgccatgcacatagcttgcggccgggctcatcaaaaaggcgatgaccgacgccatctcggacggctcggcacggcggcccatgggaggaacgaacttggcaatggattcgccatagcgcgggtcctgcaggcccgcctgcagcaagggagtctcggttgcaccgggggcgatggtgttcaggcgcacgccagcctcgccccaggcggcggcgcgtttgcgcacagccaccgtcaaagcattcttgctgcccgcataggccagatttccgccctgctctcccgcatgttcgacaatggcgcgggccttggcttcctcgccggcttccagtgccagcgccagtgggttcttgtcaaaagccagatgcgcggaagccacggacgagatgacgacggctgcgggctgatggccttttttcagcgctggcaaaaaggcatccatcagctcggtcgcgccaaaataattgaccgaaaccacattgccaagcaccttggtctgcggtcccaggccggcgcacagcaccaggccgtccatgcccttgctgcacttcgccagtacatcggcaatcgcctgctttcgaccttcggccgtcgagagatcggcaatcacttccgcatcgcgtatatcgatgcctacgatctggtgaccggccgcctccaggaccttgcgcgtagccgcaccaatgccggtggcgcagccgcttatcacgatgatggacatgtatatctcctttcacgaattctcagccgccttcttgaacttggcggcctcttccgaaccgccggtggcattgcccttcgagtaggaatgcgcgccggtgccggcaagagcgccgccctgcacgatgaggtattcgtcgcggatcggacggccctcgaagaagcactccaggatctcgcgggtgcccgccgcataacgcgcctgcgcggtcagcgtggtgccggagatgtgcggggtcatgccgttatagggcatcgtccgccaggggtggtccttcggcgccggctgcgggaaccacacgtcgccggcatagccggccagccggccggattcgagcgcacgtgccacggcatcgcggtcgcacagcttgccgcgggcggtgttgacgatgtaggcgccacgcttgaacagcttcagcgtctcgtcattgatcatgtgctcggtttcggggtgcagcgggcagttcagcgtcaccacgtcgcaaaccggatacatgtcctcgcgggtcgcgtgccaggtgaggttgagctccttctcgaccgattccggcaggcggtgacggtcggtgtagtgcaggtgcacgtcgaacggcgccagacggcgcagcaccgcgagaccgatgcggccggcggccacggtgccgacatgcatcgcctcgaggtcgtaggcgtgggagacgcagtcggcgatgttccagccgcccttccgcgcccattcgtgcgagggcagatagttgcgcaccagcgacaggatcatcatcaccacatgctcggcgacgctgatcgagttgcagtaggtgacttccgccacggtgacgttgcggtcgatagccgactgaagatcgacgtggtcggaaccgatgccggcggtgagcgcgagcttcaggttcttggccttggcgatgcgctcgggcgtcagataggccggccagaagggctgggagatgacgacatccgcatcgaccagctcgcgctcgaacaccgagtcggggccgtccttgtcggaggtcacgaccagggtgtggccgttggattcgagatattcgcgcaggccgagctcgccggagacggagccgagcaactgcccgggcgtgaagtcgatggccttcggcgtcggcaagatctggccgcccggatagtggtcgatcttcggaagatcgtcgcgggcataggtcttcgggtagccgtcgaccggatcatcgtaaagaacgcacaggacctttgccatcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 70 - protein, 263 aa, Collinsella aerofaciens; identical to SEQ ID NO: 2

SEQ ID NO: 71 - protein, 257 aa, Comamonas testosteroni [sequence omitted]

SEQ ID NO: 72 - protein, 322 aa, Rattus norvegicus; identical to SEQ ID NO: 8

(19) United States
(12) Patent Application Publication
(10) Pub. No.: US 2024/0240158 A1
(43) Pub. Date: Jul. 18, 2024

(54) NOVEL 7Beta-HYDROXYSTEROID DEHYDROGENASE MUTANTS AND PROCESS FOR THE PREPARATION OF URSODEOXYCHOLIC ACID

(71) Applicant: PharmaZell GmbH, Raubling (DE)

(72) Inventors: Dirk Weuster-Botz, Munich (DE); Michael Braun, Bad Mergentheim-Edelfingen (DE); Arno Aigner, Tuntenhausen (DE); Boqiao Sun, Munich (DE); Christina Kantzow, Munich (DE); Sven Bresch, Munich (DE); Daniel Bakonyi, Köln (DE); Werner Hummel, Titz (DE)

(73) Assignee: PharmaZell GmbH, Raubling (DE)

(21) Appl. No.: 18/423,606

(22) Filed: Jan. 26, 2024

Related U.S. Application Data
(62) Division of application No. 17/150,243, filed on Jan. 15, 2021, now Pat. No. 11,981,935.
Application No. 17/150,243 is a division of application No. 15/093,570, filed on Apr. 7, 2016, now Pat. No. 10,954,494.
Application No. 15/093,570 is a continuation of application No. 13/993,235, filed on Nov. 19, 2013, now abandoned.

(30) Foreign Application Priority Data
Dec. 16, 2010 (EP) 10015726

(51) Int. Cl. C12N 9/04; C12N 9/02; C12P 33/00; C12P 33/02

(52) U.S. Cl. CPC C12N 9/0006; C12N 9/0008; C12P 33/00; C12P 33/02; C12Y 101/01201

(57) ABSTRACT

In various aspects and embodiments, the invention provides a nucleic acid molecule comprising a nucleotide sequence encoding a 7β-hydroxysteroid dehydrogenase (7β-HSDH) mutant that catalyzes at least the stereospecific enzymatic reduction of a 7-ketosteroid to the corresponding 7-hydroxysteroid, wherein the mutant has, compared to the wildtype 7β-HSDH of SEQ ID NO:2, a decreased substrate inhibition and/or an altered cofactor usage, and the mutant has, in comparison with the wildtype 7B-HSDH of SEQ ID NO:2, 1 to 15 amino acid additions, substitutions, deletions and/or inversions in the sequence motif VMVGRRE corresponding to positions 36 to 42 of SEQ ID NO:2.

This application is a divisional under 35 U.S.C. § 121 of U.S. Ser. No. 17/150,243, filed Jan. 15, 2021, which is a divisional under 35 U.S.C. § 121 of U.S. Ser. No. 15/093,570, filed Apr. 7, 2016, now U.S. Pat. No. 10,954,494, issued Mar. 23, 2021, which is a continuation under 35 U.S.C. § 120 of U.S. Ser. No. 13/993,235, filed Nov. 19, 2013, now abandoned, which is the U.S. national phase pursuant to 35 U.S.C. § 371 of PCT international application No. PCT/EP2011/073141, filed Dec. 16, 2011, which claims priority to EP patent application No. 10015726, filed Dec. 16, 2012. The entire contents of each of the aforementioned patent applications are incorporated herein by this reference.

The invention relates to novel 7β-hydroxysteroid dehydrogenase mutants, to the sequences that code for these enzyme mutants, to processes for the preparation of the enzyme mutants and use thereof in enzymatic reactions of cholic acid compounds, and especially in the preparation of ursodeoxycholic acid (UDCA); the invention also relates to novel processes for the synthesis of UDCA using the enzyme mutants; and to the preparation of UDCA using recombinant, multiply-modified microorganisms.

### SEQUENCE LISTING

The instant application contains a Sequence Listing which has been submitted electronically in ASCII format and is hereby incorporated by reference in its entirety. Said ASCII copy, created on Feb. 13, 2024, is named Reitstotter 054110-7038US4-DIV2—Corrected Sequence Listing ST.26.xml and is 201,886 bytes in size.

### BACKGROUND OF THE INVENTION

The active substances ursodeoxycholic acid (UDCA) and the related diastereomer chenodeoxycholic acid (CDCA), among others, have been used for many years for the drug treatment of gallstone disease. The two compounds differ only in the configuration of the hydroxyl group on carbon atom 7 (UDCA: β-configuration, CDCA: α-configuration). Various processes are described in the prior art for the preparation of UDCA, which are carried out purely chemically or consist of a combination of chemical and enzymatic process steps. The starting point is in each case cholic acid (CA) or CDCA prepared from cholic acid.

Thus, the classical chemical method for UDCA preparation can be represented schematically as shown in FIG. 27.

A serious disadvantage is, among other things, the following: as the chemical oxidation is not selective, the carboxyl group and the 3a and 7α-hydroxyl group must be protected by esterification.

An alternative chemical/enzymatic process based on the use of the enzyme 12α-hydroxysteroid dehydrogenase (12α-HSDH) can be represented as shown in FIG. 28 and is for example described in PCT/EP2009/002190 of the present applicant.

The 12α-HSDH oxidizes CA selectively to 12-keto-CDCA. The two protection steps required according to the classical chemical method are then omitted.

Furthermore, Monti, D., et al., (One-Pot Multienzymatic Synthesis of 12-Ketoursodeoxycholic Acid: Subtle Cofactor Specificities Rule the Reaction Equilibria of Five Biocatalysts Working in a Row. Advanced Synthesis & Catalysis, 2009) describe an alternative enzymatic-chemical process, which can be represented schematically as shown in FIG. 29.

The CA is first oxidized from 7α-HSDH from Bacteroides fragilis ATCC 25285 (Zhu, D., et al., Enzymatic enantioselective reduction of-ketoesters by a thermostable 7-hydroxysteroid dehydrogenase from Bacteroides fragilis. Tetrahedron, 2006. 62(18): p. 4535-4539) and 12α-HSDH to 7,12-diketo-LCA. These two enzymes are each NADH-dependent. After reduction by 7β-HSDH (NADPH-dependent) from Clostridium absonum ATCC 27555 (DSM 599) (MacDonald, I. A. and P. D. Roach, Bile induction of 7 alpha- and 7 beta-hydroxysteroid dehydrogenases in Clostridium absonum. Biochim Biophys Acta, 1981. 665(2): p. 262-9), 12-keto-UDCA is formed. The end product is obtained by Wolff-Kishner reduction. This method has the drawback that owing to the position of the equilibrium of the catalyzed reaction, a complete reaction is not possible, and that for the first step of the reaction it is necessary to use two different enzymes, which makes the process more expensive. For cofactor regeneration, lactate dehydrogenase (LDH; for regeneration of NAD⁺) and glucose dehydrogenase (GlcDH or GDH, for regeneration of NADPH) are used. A disadvantage with the cofactor regeneration used there is that the resultant co-product can only be removed from the reaction mixture with great difficulty, so that the reaction equilibrium cannot be influenced positively, which results in incomplete reaction of the educt.

A 7β-HSDH from the strain Collinsella aerofaciens ATCC 25986 (DSM 3979; formerly Eubacterium aerofaciens) was described in the year 1982 by Hirano and Masuda (Hirano, S. and N. Masuda, Characterization of NADP-dependent 7 beta-hydroxysteroid dehydrogenases from Peptostreptococcus productus and Eubacterium aerofaciens. Appl Environ Microbiol, 1982. 43(5): p. 1057-63). Sequence information for this enzyme was not disclosed. The molecular weight determined by gel filtration was 45 000 Da (cf. Hirano, page 1059, left column). Furthermore, for the enzyme there, the reduction of the 7-oxo group to the 7β-hydroxyl group was not observed (cf. Hirano, page 1061, Discussion 1st paragraph). A person skilled in the art can therefore see that the enzyme described by Hirano et al. is not suitable for catalysis of the reduction of dehydrocholic acid (DHCA) in position 7 to 3,12-diketo-7β-CA.

The applicant's earlier international patent application PCT/EP2010/068576 describes a novel 7β-HSDH from Collinsella aerofaciens ATCC 25986, which among other things has a molecular weight (in SDS-gel electrophoresis) of about 28-32 kDa, a molecular weight (in gel filtration, in nondenaturing conditions, such as in particular without SDS) from about 53 to 60 kDa, and the capacity for stereoselective reduction of the 7-carbonyl group of 7-keto-LCA to a 7β-hydroxyl group.

In addition, in PCT/EP2010/068576, a process is provided for the preparation of UDCA, which can be represented schematically as shown in FIG. 30.

In this case the oxidation of CA takes place simply, by a classical chemical route. DHCA is reduced by the pair of enzymes 7-HSDH and 3α-HSDH individually in succession or in one pot to 12-keto-UDCA. Combined with Wolff-Kishner reduction, UDCA can therefore be synthesized from CA in just three steps. 7β-HSDH is dependent on the cofactor NADPH, whereas 3α-HSDH requires the cofactor NADH. The availability of pairs of enzymes with dependence on the same cofactor or extended dependence (e.g. on the cofactors NADH and NADPH) would be advantageous, because this could simplify cofactor regeneration.

The problem to be solved by the invention is to provide further improved 7β-HSDHs. In particular, enzyme mutants should be provided, which can be used even more advantageously for enzymatic or microbial preparation of UDCA via the stereospecific reduction of DHCA in 7-position to 3,12-diketo-7β-CA, and in particular have reduced substrate inhibition and/or have altered cofactor usage (increased, altered specificity or extended dependence).

Another problem is to provide novel enzymatic and microbial synthesis routes, which in particular are also characterized by simplified cofactor regeneration in the reductive preparation of UDCA via DHCA.

### SUMMARY OF THE INVENTION

The above problems were solved, surprisingly, by the production and characterization of mutants of a novel regio- and stereospecific 7β-HSDH from aerobic bacteria of the genus Collinsella, especially of the strain Collinsella aerofaciens and use thereof in the reaction of cholic acid compounds, especially in the preparation of UDCA.

Furthermore, the above problem was solved by providing a biocatalytic (microbial or enzymatic) process, comprising the enzymatic conversion of DHCA via two partial reductive steps catalyzed by 7β-HSDH or 3α-HSDH, which can take place simultaneously or with a time delay in any order, to 12-keto-UDCA and cofactor regeneration using dehydrogenases, such as in particular formate dehydrogenase (FDH) enzymes or glucose dehydrogenase (GDH) enzymes, which regenerate the spent cofactor from the two partial reductive steps.

### DESCRIPTION OF THE FIGURES

FIG. 1A shows the amino acid sequence of 7β-HSDH from Collinsella aerofaciens and FIG. 1B shows the coding nucleic acid sequence for the amino acid sequence of FIG. 1A; FIG. 1C shows the amino acid sequence of 3α-HSDH from Comanomonas testosteroni and FIG. 1D shows the coding nucleic acid sequence for the amino acid sequence of FIG. 1C; FIG. 1E shows the amino acid sequence of 3α-HSDH from Rattus norvegicus and FIG. 1F shows the coding nucleic acid sequence for the amino acid sequence of FIG. 1E; FIG. 1G shows the coding nucleic acid sequence of the FDH mutant D221G and FIG. 1H shows the amino acid sequence for the nucleic acid sequence of FIG. 1G.

FIG. 2 shows the SDS-gel of a purified 7β-HSDH prepared according to the invention, with, on lane 1: cell raw extract; lane 2: purified protein; lane M: Page Rouler™, molecular weight marker (Fermentas, Germany).

FIG. 3A, FIG. 3B, FIG. 3C and FIG. 3D shows the construction schemes of (A) pET21a(+), pET22b(+), pCOLA (Mod) and pET28a(+), respectively.

FIG. 4A and FIG. 4B show the construction schemes of pET21a(+) FDH D221G and pET21a(+) FDH 7β-HSDH, respectively.

FIG. 5 shows the construction scheme of pCOLA(mod) 3α-HSDH.

FIGS. 6A and 6B show an activity comparison for 7β-HSDH wild type and the 7β-HSDH mutants G39A and G39S; specifically. FIG. 6A is a plot of the specific enzyme activity versus different substrate concentrations used at constant cofactor concentration of 100 μM. FIG. 6B is a plot of enzyme activity versus different cofactor concentrations used at constant substrate concentration of 0.3 mM.

FIG. 7 shows the HPLC chromatogram of the biotransformation of DHCS with 7β-HSDH and 3α-HSDH in the whole-cell process. It shows the HPLC chromatogram of the whole-cell conversion of the strain E. coli BL21 (DE3) hdhA⁻KanR⁺ pET21a(+) FDH 7β-HSDH pCOLA(mod) 3α-HSDH. The starting conditions selected were 50 mM substrate, 400 mM cosubstrate, pH 6.0. Sampling was carried out after 48 h. The peaks of 12-keto-UDCA (1), an unknown by-product (2), 3,12-diketo-UDCA (3), 7,12-diketo-UDCA (4) and DHCA (5) can be seen.

FIG. 8 shows the construction scheme of the triple vector pET21a(+) FDH 7beta (G39A) that bears the coding sequences for FDH D221G, 7β-HSDH G39A and 3α-HSDH.

FIG. 9 shows the course of whole-cell biotransformation of DHCA. The proportionate peak areas (according to HPLC analysis) of 12-keto-UDCA, 3,12-diketo-UDCA, 7,12-diketo-UDCA and DHCA are shown as a function of time.

FIG. 10 shows a sequence comparison between the 7β-HSDH wild type and selected mutants. The sequences designated as “7beta-HSDH wild type”, “7beta-HSDH G39D”, “7beta-HSDH G39D R40L”, “7beta-HSDH G39D R40I” and “7beta-HSDH G39D R40V” correspond to SEQ ID NO:2, SEQ ID NO:37, SEQ ID NO:38 and SEQ ID NO:39.

FIG. 11 shows an enzyme-kinetic investigation of 7β-HSDH and the mutants thereof. The specific enzyme activity is plotted versus different substrate concentrations used (DHCA concentration) at a constant cofactor concentration of 0.1 mM NADPH or 0.5 mM NADH.

FIG. 12 shows a schematic representation of a two-step enzymatic reduction of dehydrocholic acid to 12-keto-ursodeoxycholic acid according to the invention, wherein an NADH-dependent 7β-HSDH is used and a formate dehydrogenase (FDH) is used for cofactor regeneration.

FIG. 13 shows a schematic representation of the principle of construction of the vectors pFr7(D), pFr7(DI), pFr7(DL) and pFr7(DV) that were used for the expression of various NADH-dependent mutants of 7β-HSDH.

FIG. 14 shows the proportions of bile salts in biotransformation batches using NADH-dependent 7β-HSDH mutants. Results after 24 h process time and using 100 mM substrate (DHCA) are shown.

FIG. 15 shows a schematic representation of the vector pFr3T7(D).

FIG. 16 shows the result of a whole-cell biotransformation with the strain E. coli BL49 pFr3T7(D), which comprises the NADH-dependent 7β-HSDH (G39D), an NADH-dependent 3α-HSDH and an NADH-dependent FDH. The biotransformations were carried out in the following conditions: 20 mL reaction volume, 17.7 g/l_{BTM} cells, 100 mM DHCA, 500 mM ammonium formate, 26% glycerol, 50 mM MgCl₂, 50 mM KPi buffer (pH 6.5). During the first 5 hours the pH was adjusted manually with formic acid to the initial value at hourly intervals.

FIG. 17A shows a schematic representation of vector pF(G)r7(A)r3 which corresponds to the vector shown in FIG. 8) and FIG. 17B shows a schematic representation of vector pF(G)r7(S)r3.

FIG. 18 shows a schematic representation of the two-step enzymatic reduction of dehydrocholic acid to 12-keto-ursodeoxycholic acid, wherein an NADPH-dependent 7β-HSDH is used and a formate dehydrogenase (FDH) mutant, which regenerates both NADPH- and NADH, is used for cofactor regeneration.

FIG. 19 shows a time-resolved variation of the biotransformation with the strain E. coli BL49 pF(G)r7(A)r3 at the liter scale. The batch contained 70 mM DHCA, 17.7 g/l BTM of the stored biocatalyst, 500 mM sodium formate, 26% (v/v) glycerol, 50 mM MgCl₂, in 50 mM KPi buffer (pH 6.5). Using pH adjustment, the pH was maintained at pH 6.5 throughout the biotransformation.

FIG. 20 shows a schematic representation of the vector p3T7(A)rG.

FIG. 21 shows a schematic representation of the vector p7(A)T3rG.

FIG. 22A and FIG. 22B show a time-resolved variation of the biotransformation with the strain E. coli BL49 p7(A)T3rG, which comprises a GDH for cofactor regeneration. The batch contained 100 mM DHCA, 17.7 g/l BTM of the stored biocatalyst, 500 mM glucose, 10 mM MgCl₂, in 50 mM KPi buffer (pH 7) without (FIG. 22A) and with 0.1 mM NAD (FIG. 22B). The pH was adjusted manually with potassium hydroxide solution to the initial value.

FIG. 23A shows a schematic representation of vector pF(G)r7(A) and FIG. 23B shows a schematic representation of vector pFr3.

FIG. 24 shows a schematic representation of the two-step enzymatic reduction of dehydrocholic acid to 12-keto-ursodeoxycholic acid using two different whole-cell biocatalysts. Reactions A and D are catalyzed by a 7β-HSDH containing whole-cell biocatalyst, and reactions B and C are catalyzed by a 3α-HSDH containing whole-cell biocatalyst. In the course of the two-step reaction, the intermediate 3,12-diketo-ursodeoxycholic acid must pass from the 7β-HSDH containing whole-cell biocatalyst into the 3α-HSDH containing whole-cell biocatalyst, and the intermediate 7,12-diketo-ursodeoxycholic acid must pass from the 3α-HSDH containing whole-cell biocatalysts into the 7β-HSDH containing whole-cell biocatalyst.

FIG. 25 shows the proportions of bile salts of biotransformation batches after 24 h when using 70 mM substrate (DHCA). The batches are shown when using different proportions of the two biocatalyst strains E. coli BL49 pF(G)r7(A) and E. coli BL49 pFr3.

FIG. 26 shows a time-resolved variation of biotransformation with the biocatalysts E. coli BL49 pF(G)r7(A) and E. coli BL49 pFr3 liter scale. The batch contained 90 mM DHCA, 8.85 g/l BTM E. coli BL49 pF(G)r7(A) and 8.85 g/l BTM E. coli BL49 pFr3, 500 mM ammonium formate, 26% (v/v) glycerol, 50 mM MgCl₂, in 50 mM KPi buffer (pH 6.5). Using pH adjustment, the pH was maintained with formic acid at pH 6.5 throughout the biotransformation.

FIG. 27 shows a flow chart of a chemical transformation pathway relevant to UDCA.

FIG. 28 shows an example of a chemical transformation pathway relevant to UDCA.

FIG. 29 shows an alternative example of a chemical transformation pathway relevant to UDCA.

FIG. 30 shows an example of a process for preparation of UDCA.

FIG. 31 shows the chemical transformation pathway of reaction example 1.

FIG. 32 shows the chemical transformation pathway of reaction example 2.

FIG. 33 shows the chemical transformation pathway of reaction example 3.

FIG. 34 shows an example of a reaction scheme involving NADPH as a cofactor regeneration system.

FIG. 35 shows an example of a reaction scheme involving NADH as a cofactor regeneration system.

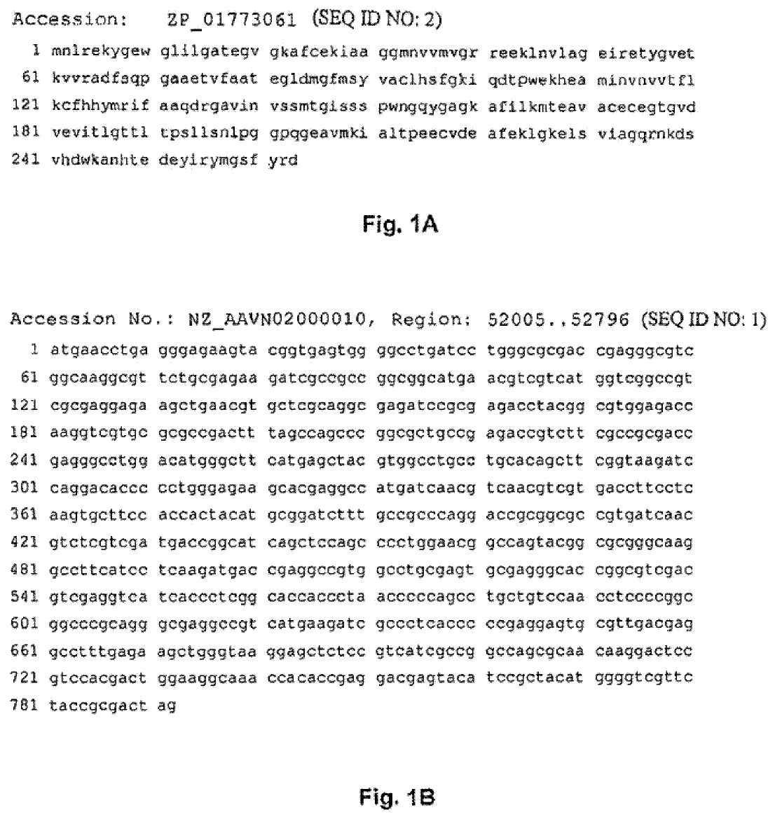

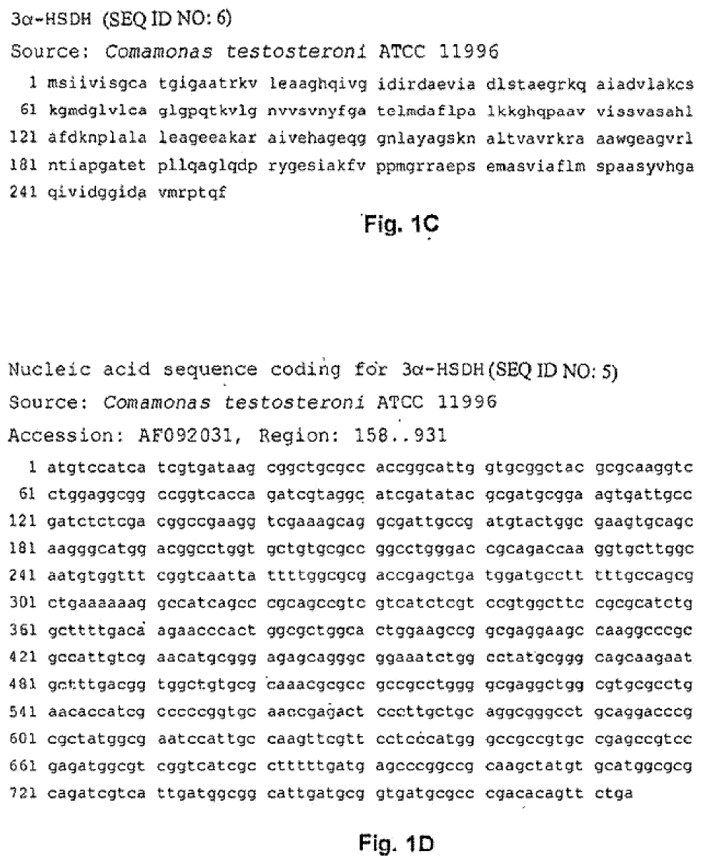

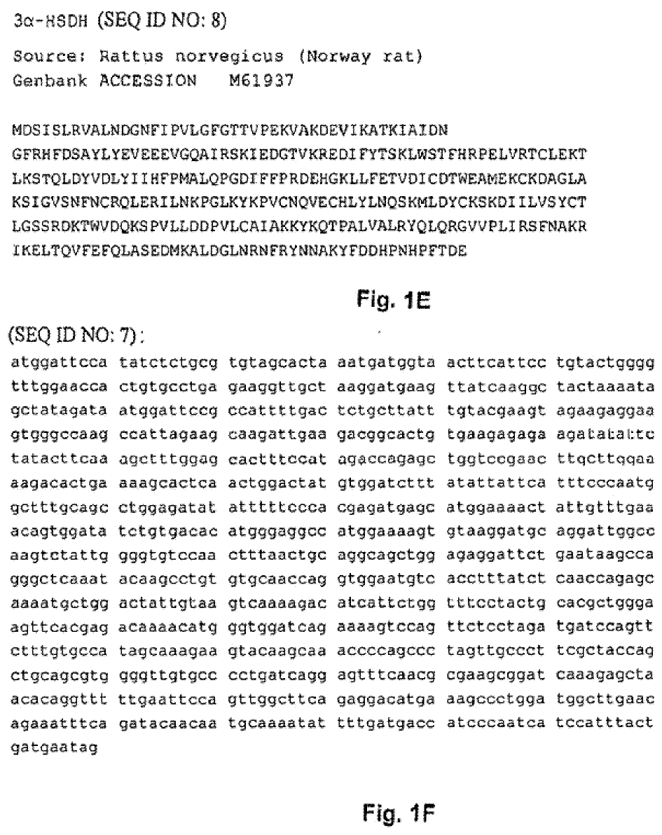

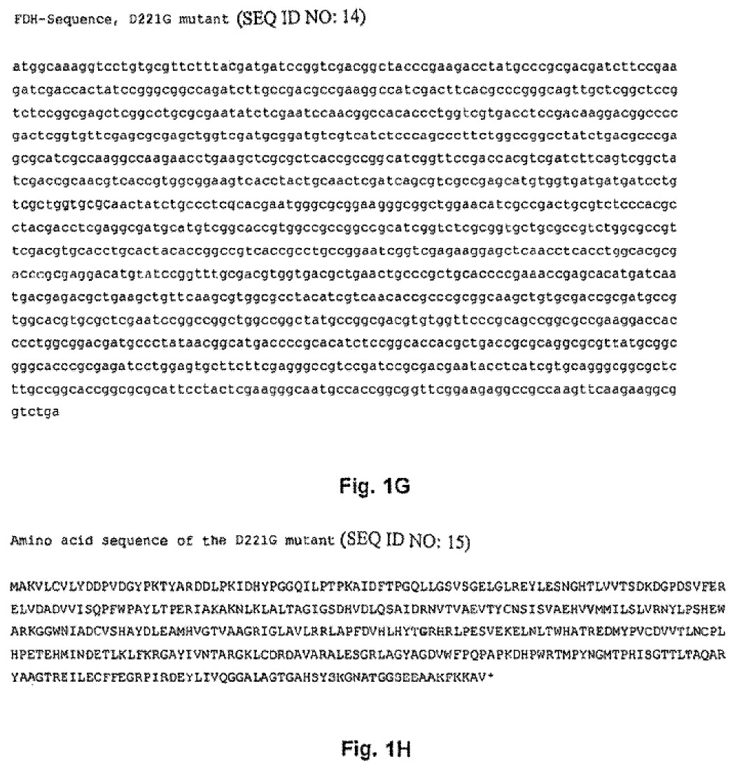

### SPECIAL EMBODIMENTS OF THE INVENTION

The invention relates in particular to the following special embodiments:
1. A 7β-hydroxysteroid dehydrogenase (7β-HSDH) mutant, which catalyzes at least the stereospecific enzymatic reduction of a 7-ketosteroid to the corresponding 7-hydroxysteroid, wherein the mutant has a decreased substrate inhibition (especially for the 7-ketosteroid substrate) compared to the unmutated enzyme, such as in particular an enzyme comprising SEQ ID NO:2 and/or at least the sequence motif VMVGRRE according to position 36 to 42 thereof; and/or an altered cofactor usage (e.g. increased, altered specificity with respect to a cofactor (especially NADH or NADPH) or an extended dependence, i.e. usage of an additional cofactor not used previously).
2. Mutant according to embodiment 1, which does not display any substrate inhibition or which has a Kᵢ value for the 7-ketosteroid substrate, especially for dehydrocholic acid (DHCA), in the range from >10 mM, e.g. at 11 to 200 mM, 12 to 150 mM, 15 to 100 mM.
3. Mutant according to one of the preceding embodiments, wherein the specific activity (U/mg) in the presence of the cofactor NADPH, compared to the unmutated enzyme, is raised or lowered by at least 1, 5 or 10%, but especially at least 1-fold, especially 2- to 10-fold; or essentially is absent and is replaced by the usage of NADH, or has an extended usage of NADPH and HADH.
4. A 7β-hydroxysteroid dehydrogenase (7β-HSDH) mutant, which catalyzes at least the stereospecific enzymatic reduction of a 7-ketosteroid to the corresponding 7-hydroxysteroid, optionally according to one of the preceding embodiments, wherein the mutant has at least one mutation in the amino acid sequence according to SEQ ID NO:2 or an amino acid sequence derived therefrom with at least 60% sequence identity, e.g. at least 65, 70, 75, 80, 85, or 90, e.g. at least 91, 92, 93, 94, 95, 96, 97, 98, 99 or 99.5% to this sequence.
5. A 7β-hydroxysteroid dehydrogenase (7β-HSDH) mutant, which catalyzes at least the stereospecific enzymatic reduction of a 7-ketosteroid to the corresponding 7-hydroxysteroid, optionally according to one of the preceding embodiments, wherein the mutant has at least one mutation in the sequence motif VMVGRRE according to position 36 to 42 of SEQ ID NO:2 or in the corresponding sequence motif of an amino acid sequence derived therefrom with at least 60% sequence identity, e.g. at least 65, 70, 75, 80, 85, or 90, e.g. at least 91, 92, 93, 94, 95, 96, 97, 98, 99 or 99.5% to this sequence.
6. Mutant according to embodiment 4 or 5, selected from
a) the single mutants G39X₁ and R40X₂,
b) the double mutants (G39X₁, R40X₂), (R40X₂, R41X₃) and (G39X₁, R41X₃) or
c) the triple mutant (G39X₁, R40X₂, R41X₃),
(in each case relative to SEQ ID NO:2)
wherein X₁, X₂ and X₃ in each case independently of one another stand for any amino acid, especially any, especially natural, amino acid, different from G or R, that decreases substrate inhibition and/or modifies cofactor usage or cofactor dependence;
or
d) the corresponding single, double or triple mutants of an amino acid sequence derived from SEQ ID NO:2 with at least 60% sequence identity, e.g. at least 65, 70, 75, 80, 85, or 90, e.g. at least 91, 92, 93, 94, 95, 96, 97, 98, 99 or 99.5% to this sequence.
   Examples of suitable single mutants comprise: G39A, G39S, G39D, G39V, G39T, G39P, G39N, G39E, G39Q, G39H, G39R, G39K and G39W, and R40D, R40E, R40I, R40V, R40L, R40G, R40A.
   Examples of suitable double mutants comprise:
double combinations of the above G39X₁ and R40X₂ mutants, wherein X₁ stands in particular for D or E and/or X₂ can be any amino acid, especially proteinogenic amino acid, such as in particular an amino acid with aliphatic side chain, for example (G39D,R40I), (G39D,R40L), (G39D,R40V); and the analogous double mutants with G39E instead of G39D; and
double combinations of the above G39X₁ mutants or R40X₂ mutants with R41X₃ mutants, in which X₃ is any amino acid, especially an, especially natural, amino acid, that decreases substrate inhibition and/or that modifies cofactor usage or cofactor dependence, different from R, for example of the type (G39X₁, R41X₃) or (R40X₂,R41X₃),
e.g. with X₁=D or E; X₂₌₁, L or V; X₃=N, I, L or V)
for example (R40D,R41I); (R40D,R41L); (R40D,R41V); (R40I,R41I); (R40V,R41I), (R40L,R41I).
   Examples of suitable triple mutants comprise any triple combinations of the above single mutants G39X₁, R40X₂ and R41X₃; wherein X₁, X₂ and X₃ are as defined above; but especially wherein X₁ stands for D, or E and/or X₂ and X₃ stand independently of one another for any amino acid, especially a proteinogenic amino acid, such as in particular triple mutants of the type (G39X₁=D or E; R40X₂₌₁, L or V; R41X₃=N, I, L or V), for example (G39D,R40I,R41N).
   Optionally the mutants of embodiments 1 to 6 can have, additionally or alternatively, especially additionally, at least one further substitution, for example 1, 2, 3 or 4 substitutions in the positions K44, R53, K61 and R64. In this case these residues can be replaced, independently of one another, with any amino acid, especially a proteinogenic amino acid, especially a substitution such that the resultant mutant has a decreased substrate inhibition (especially for the 7-ketosteroid substrate); and/or has an altered cofactor usage or cofactor dependence (e.g. increased, altered specificity with respect to a cofactor or an extended dependence, i.e. usage of an additional cofactor not used previously) as defined herein.
7. Nucleic acid sequence coding for a 7β-HSDH mutant according to one of the preceding embodiments.
8. Expression cassette, comprising a nucleic acid sequence according to embodiment 7 under the genetic control of at least one regulatory nucleic acid sequence, and optionally coding sequences for at least one (for example 1, 2 or 3) further enzyme, selected from hydroxysteroid dehydrogenases, especially 3α-HSDH, and dehydrogenases suitable for cofactor regeneration, for example FDH, GDH, ADH, G-6-PDH, PDH. In particular the enzymes contained in an expression cassette can use different, but preferably the same pairs of cofactors, for example the pair of cofactors NAD⁺/NADH or NADP⁺/NADPH.
9. Vector comprising at least one expression cassette according to embodiment 8.
10. Recombinant microorganism, bearing at least one nucleic acid sequence according to embodiment 7 or at least one expression cassette according to embodiment 8 or bearing at least one vector according to embodiment 9 and additionally, optionally, bearing the coding sequence for a 3α-hydroxysteroid dehydrogenase (3α-HSDH).
11. Process for the enzymatic or microbial synthesis of 7β-hydroxysteroids, wherein the corresponding 7-ketosteroid is reacted in the presence of a 7β-HSDH mutant according to the definition in one of the embodiments 1 to 6 or in the presence of a recombinant microorganism expressing this mutant according to embodiment 10, and at least one resultant reduction product is optionally isolated from the reaction mixture.
12. The process according to embodiment 11, wherein the ketosteroid to be reduced is selected from
   a) dehydrocholic acid (DHCA),
   b) 7-keto-lithocholic acid (7-keto-LCA),
   c) 7,12-diketo-lithocholic acid (7,12-diketo-LCA) and
   d) derivatives thereof, such as in particular a salt, amide or alkyl ester of the acid.
13. The process according to one of the embodiments 11 and 12, wherein the reduction takes place in the presence of (and with the consumption of) NADPH and/or NADH.
14. A process for enzymatic or microbial oxidation of 7β-hydroxysteroids, wherein the hydroxysteroid is reacted in the presence of a 7β-HSDH mutant according to the definition in one of the embodiments 1 to 6 or in the presence of a microorganism expressing this mutant according to embodiment 10, and a resultant oxidation product is optionally isolated from the reaction mixture.
15. The process according to embodiment 14, wherein the 7β-hydroxysteroid is 3,12-diketo-7β-CA or a derivative thereof, such as in particular a salt, amide or alkyl ester.
16. The process according to one of the embodiments 14 and 15, wherein the oxidation takes place in the presence of (and with the consumption of) NADP⁺ and/or NAD⁺.
17. The process according to one of the embodiments 13 and 16, wherein the spent redox equivalents are regenerated chemically, electrochemically or enzymatically, especially in situ.
18. The process according to embodiment 17, wherein spent NADPH is regenerated by coupling with an NADPH-regenerating enzyme, wherein this is selected in particular from NADPH dehydrogenases, alcohol dehydrogenases (ADH), and NADPH regenerating formate dehydrogenases (FDH), and glucose dehydrogenase (GDH)-, glucose-6-phosphate dehydrogenase (G-6-PDH), or phosphite dehydrogenases (PtDH), wherein the NADPH-regenerating enzyme is optionally expressed by a recombinant microorganism; or wherein spent NADH is regenerated by coupling with an NADH-regenerating enzyme, wherein this is selected in particular from NADH dehydrogenases, NADH regenerating formate dehydrogenases (FDH), NADH regenerating alcohol dehydrogenases (ADH), NADH regenerating glucose-6-phosphate dehydrogenases (G6PDH), NADH regenerating phosphite dehydrogenases (PtDH) and NADH regenerating glucose dehydrogenases (GDH), wherein the NADH-regenerating enzyme is optionally expressed in a recombinant microorganism.
   Expression of the cofactor-regenerating enzyme in a recombinant microorganism is preferred.
19. The process according to embodiment 18, wherein the NADPH-regenerating enzyme is selected from natural or recombinant, isolated or enriched
   a) alcohol dehydrogenases (EC 1.1.1.2) and
   b) functional equivalents derived therefrom.
20. The process according to embodiment 18, wherein the NADPH-regenerating enzyme is selected from mutants of an NAD⁺-dependent formate dehydrogenase (FDH), which in particular catalyzes at least the enzymatic oxidation of formic acid to CO₂, wherein the mutant accepts, compared to the unmutated enzyme, exclusively or additionally, especially additionally, NADP⁺ as cofactor; or
wherein the NADH-regenerating enzyme is selected from an NAD⁺-dependent FDH or an NAD⁺-dependent GDH, such as in particular an FDH from Mycobacterium vaccae N10, according to SEQ ID NO:36 and a GDH from Bacillus subtilis according to SEQ ID NO:48 (which regenerates NADPH and/or NADH) or a modified form thereof in each case functionally equivalent (with respect to cofactor regeneration) to the two stated enzymes.
21. The process according to embodiment 20, wherein the FDH mutant reduces NADP⁺ with a specific activity to NADPH, which corresponds to about 0.1 to 1000%, such as 1 to 100%, 5 to 80% or 10 to 50%, of the specific activity of the unmutated (wild-type) enzyme for the reduction of NAD⁺ to NADH.
22. The process according to one of the embodiments 20 and 21, wherein the NADP⁺-accepting FDH mutant has at least one mutation in the amino acid sequence of an FDH from Mycobacterium vaccae N10 according to SEQ ID NO:36 or an amino acid sequence derived therefrom with at least 60% sequence identity, e.g. at least 65, 70, 75, 80, 85, or 90, e.g. at least 91, 92, 93, 94, 95, 96, 97, 98, 99 or 99.5% to this sequence.
23. The process according to one of the embodiments 20 to 22, wherein the NADP⁺-accepting mutant has at least one mutation in the sequence motif TDRHRL according to position 221 to 226 of SEQ ID NO:36 or in the corresponding sequence motif of an amino acid sequence derived therefrom with at least 60% sequence identity, e.g. at least 65, 70, 75, 80, 85, or 90, e.g. at least 91, 92, 93, 94, 95, 96, 97, 98, 99 or 99.5% to this sequence.
   Non-limiting examples of possibly suitable FDH mutants comprise mutations in the positions D222 and/or R223 of SEQ ID NO:36. As examples we may mention D222X with X=G, A, K or N; and R223X with X=H or Y, and combinations of mutations in position 222 and 223.
24. The process according to one of the embodiments 20 to 23, wherein the NADP⁺-accepting mutant is selected from the single mutant D222G according to SEQ ID NO:36 (herein also more often designated as “D221G” mutant (with counting, starting from Ala in position 2 of SEQ ID NO:36 as first amino acid) or the corresponding single mutants of an amino acid sequence derived therefrom with at least 60% sequence identity, e.g. at least 65, 70, 75, 80, 85, or 90, e.g. at least 91, 92, 93, 94, 95, 96, 97, 98, 99 or 99.5% to this sequence. As concrete examples, we may mention FDH mutants according to SEQ ID NO: 15, 19 and 35.
   Further suitable FDH enzymes are accessible starting from the wild-type enzymes that can be isolated e.g. from Candida boidinii or Pseudomonas sp, and insertion of at least one functional mutation corresponding to the above mutations for altering the cofactor specificity. Moreover, there have been numerous studies in the prior art for improving various FDH properties, such as chemical or thermal stability or catalytic activity. These are summarized e.g. in Tishkov et al., Biomolecular Engineering 23 (2006), 89-110. Thus, single or multiple point mutations described there can be combined, for example for increasing enzyme stability, with the mutations described according to the invention for modified cofactor usage.
25. A nucleic acid sequence, selected from nucleic acid sequences a) simultaneously coding for an FDH mutant according to one of the embodiments 22 to 24 and a 7β-HSDH mutant according to one of the embodiments 1 to 6 and optionally a 3α-HSDH; or b) simultaneously coding for an FDH mutant according to one of the embodiments 22 to 24 and an unmutated 7β-HSDH and optionally a 3α-HSDH; or c) coding for a fusion protein comprising an FDH mutant according to one of the embodiments 22 to 24 and a 7β-HSDH mutant according to one of the embodiments 1 to 6 and optionally a 3α-HSDH; or d) coding for a fusion protein comprising an FDH mutant according to one of the embodiments 22 to 24 and an unmutated 7β-HSDH and optionally a 3α-HSDH, e) simultaneously coding for FDH wild type and a 7β-HSDH mutant according to one of the embodiments 1 to 6 and optionally a 3α-HSDH; or f) coding for a fusion protein, comprising the FDH wild type, a 7β-HSDH mutant according to one of the embodiments 1 to 6 and optionally a 3α-HSDH; g) simultaneously coding for a GDH, a 7β-HSDH wild type and optionally a 3α-HSDH; h) coding for a fusion protein, comprising a GDH, a 7β-HSDH wild type and optionally a 3α-HSDH; i) simultaneously coding for a GDH, a 7β-HSDH mutant according to one of the embodiments 1 to 6 and optionally a 3α-HSDH; and k) coding for a fusion protein, comprising a GDH, a 7β-HSDH mutant according to one of the embodiments 1 to 6 and optionally a 3α-HSDH;
wherein the coding sequences can be contained, independently of one another, singly or multiply in the construct, for example in 2, 3, 4, 5, or 6 to 10 copies. Through selection of the appropriate copy number, optionally occurring activity differences in the individual expression products can be compensated.
26. Expression cassette, comprising a nucleic acid sequence according to embodiment 25 under the genetic control of at least one regulatory nucleic acid sequence, wherein the coding sequences, independently of one another, can be contained singly or multiply in the construct, for example in 2, 3, 4, 5, or 6 to 10 copies. Through selection of the appropriate copy number, optionally occurring activity differences in the individual expression products can be compensated.
27. A vector comprising at least one expression cassette according to embodiment 26, wherein the coding sequences, independently of one another, can be contained singly or multiply in the vector construct, for example in 2, 3, 4, 5, or 6 to 10 copies. Through selection of the appropriate copy number, optionally occurring activity differences in the individual expression products can be compensated.
28. A recombinant microorganism bearing at least one nucleic acid sequence according to embodiment 25 or at least one expression cassette according to embodiment 27 or bearing at least one vector according to embodiment 28.
29. A recombinant microorganism that is capable of simultaneous expression of 7β-HSDH (wild type), an NADP⁺-accepting FDH mutant and/or the corresponding FDH wild type and optionally of 3α-HSDH; or which is capable of simultaneous expression of 7β-HSDH (wild type), a GDH described herein and optionally a 3α-HSDH described herein.
30. A recombinant microorganism that is capable of simultaneous expression of a 7β-HSDH mutant, an NADP⁺-accepting FDH mutant and/or the corresponding FDH wild type and optionally of 3α-HSDH; or which is capable of simultaneous expression of a 7β-HSDH mutant, a GDH described herein and optionally a 3α-HSDH described herein.
31. The recombinant microorganism according to embodiment 30, wherein the 7β-HSDH mutant is a mutant according to one of the embodiments 1 to 6.
32. The recombinant microorganism according to embodiment 29 or 30, wherein the FDH mutant is a mutant according to the definition in one of the embodiments 20 to 24; and wherein the FDH wild type is an FDH from Mycobacterium vaccae N10 according to SEQ ID NO:36 or an FDH derived therefrom with at least 60% sequence identity, e.g. at least 65, 70, 75, 80, 85, or 90, e.g. at least 91, 92, 93, 94, 95, 96, 97, 98, 99 or 99.5% to this sequence.
33. The recombinant microorganism according to embodiment 29 or 30, wherein the 3α-HSDH is an enzyme comprising an amino acid sequence according to SEQ ID NO: 6 or 8 or 22 or an amino acid sequence derived therefrom with at least 60% sequence identity, e.g. at least 65, 70, 75, 80, 85, or 90, e.g. at least 91, 92, 93, 94, 95, 96, 97, 98, 99 or 99.5% to this sequence.
34 The recombinant microorganism according to one of the embodiments 29 to 33, bearing the coding sequences for 7β-HSDH, FDH and 3α-HSDH on one or more (different) expression constructs. The invention therefore relates to recombinant microorganisms, which are modified (for example transformed) with a single-plasmid system, bearing the coding sequences for 7β-HSDH or mutants thereof, FDH or mutants thereof and 3α-HSDH or mutants thereof in one or more copies, for example in 2, 3, 4, 5, or 6 to 10 copies. The invention therefore also relates to recombinant microorganisms, which are modified (for example transformed) with a single-plasmid system, bearing the coding sequences for 7β-HSDH or mutants thereof, GDH or mutants thereof and 3α-HSDH or mutants thereof in one or more copies, for example in 2, 3, 4, 5 or 6 to 10 copies. The enzymes (7β-HSDH, FDH and 3α-HSDH or mutants thereof) can, however, also be contained on 2 or 3 separate, mutually compatible plasmids in one or more copies. Suitable basis vectors for preparing single-plasmid systems and multicopy plasmids are known by a person skilled in the art. As examples we may mention, for single-plasmid system, e.g. pET21a and for multicopy plasmids e.g. the duet vectors marketed by the company Novagen, such as pACYCDuet-1, pETDuet-1, pCDFDuet-1, pRSFDuet-1 and pCOLADuet-1. Vectors of this kind, their compatibility with other vectors and microbial host strains are given e.g. in the “User Protocol TB340 Rev. E0305” of the company Novagen.
   The optimal combination of enzymes for the generation of plasmid systems can be undertaken by a person skilled in the art without undue effort, taking into account the teaching of the present invention. Thus, a person skilled in the art can, for example, depending on the cofactor specificity of the 7β-HSDH enzyme used in each case, select the most suitable enzyme for cofactor regeneration, selected from the aforementioned dehydrogenases, especially FDH, GDH and the respective mutants thereof.
   Furthermore, there is the possibility of distributing the enzymes selected for the reaction on two or more plasmids and, with the resultant plasmids, producing two or more different recombinant microorganisms, which are then used together for the biocatalytic reaction according to the invention. The particular enzyme combination used for preparing the plasmid can in particular also be applied specifying comparable cofactor usage. For example, a first microorganism can be modified with a plasmid, which bears the coding sequence for an NADPH-dependent 7β-HSDH mutant and an FDH mutant regenerating this cofactor according to the present invention, or which bears the coding sequence for an NADPH-dependent 7β-HSDH mutant and NADPH-regenerating GDH, or the coding sequence for an NADH-dependent 7β-HSDH and an NADH-regenerating FDH and/or GDH. A second microorganism can, in contrast, be modified with a plasmid that bears the coding sequence for an NADH-dependent 3α-HSDH and the coding sequence for an NADH-regenerating FDH wild type and/or for an NADH-regenerating GDH. Both microorganisms can then be used simultaneously for the biocatalytic reaction according to the invention.
   The use of two separate biocatalysts (recombinant microorganisms) can offer two essential advantages over the use of only one biocatalyst, in which all synthesis enzymes are expressed:
a) The two biocatalysts can be genetically modified and optimized separately from one another. In particular it is possible to use different cofactor regeneration enzymes, which are either optimized for NADH regeneration or for NADPH regeneration.
b) For the biocatalysis, the biocatalysts can be used in different proportions. This permits intervention in the individual reaction rates of the multienzyme process during biocatalysis, even after all biocatalysts have already been prepared.
   Surprisingly, it was also possible to show, in the context of the present invention, that the additional membrane transport steps of the substances that are to react, made necessary by the use of two biocatalysts, have little or no effect on the reaction rates, so that these presumed negative aspects are outweighed by the advantages of the two-cell system.
35. A process for the preparation of ursodeoxycholic acid (UDCA) of formula (1)

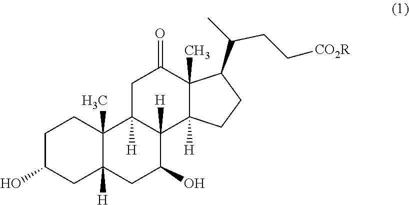

in which
R stands for alkyl, NR¹R², H, an alkali metal ion or N(R³)₄⁺, in which the residues R³ may be identical or different and stand for H or alkyl,
wherein
a) optionally a cholic acid (CA) of formula (2)

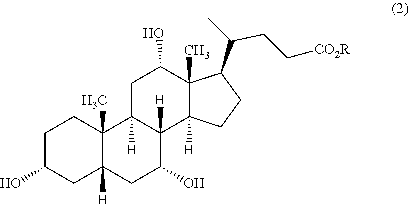

in which R has the meanings given above, is oxidized chemically to the dehydrocholic acid (DHCA) of formula (3)

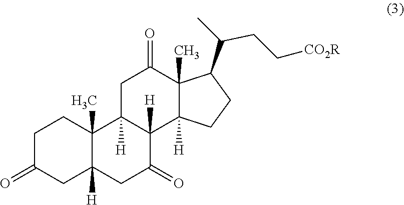

in which R has the meanings given above;
b) DHCA is reduced in the presence of at least one 7β-HSDH mutant (present as isolated enzyme or expressed by a corresponding recombinant microorganism) according to the definition in one of the embodiments 1 to 6 and in the presence of at least one 3α-hydroxysteroid dehydrogenase (3α-HSDH) (present as isolated enzyme or expressed by a corresponding recombinant microorganism) to the corresponding 12-keto-ursodeoxycholic acid (12-keto UDCA) of formula (5)

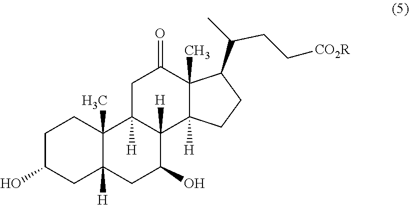

in which R has the meanings given above, (in the presence of and with consumption of NADH and/or NADPH) and then
d) 12-keto-UDCA of formula (5) is reduced chemically to UDCA; and
e) the reaction product is optionally further purified.
   Process step b) can be configured differently. Either both enzymes (7β-HSDH mutant and 3α-HSDH) can be present simultaneously (e.g. one-pot reaction with both isolated enzymes or one or more corresponding recombinant microorganisms are present, which express both enzymes), or the partial reactions can take place in any order (first the 7β-HSDH-mutant-catalyzed reduction and then the 3α-HSDH-catalyzed reduction; or first the 3α-HSDH-catalyzed reduction and then the 7β-HSDH mutant-catalyzed reduction).
   A process variant for the preparation of UDCA of formula (1) could therefore be for example as follows:
a) optionally a cholic acid (CA) of formula (2) is oxidized chemically;
b) DHCA is reduced in the presence of at least one 7β-HSDH mutant (present as isolated enzyme or expressed by a corresponding recombinant microorganism) according to the definition in one of the embodiments 1 to 6 to the 3,12-diketo-7β-cholanic acid (3,12-diketo-7β-CA) of formula (4)

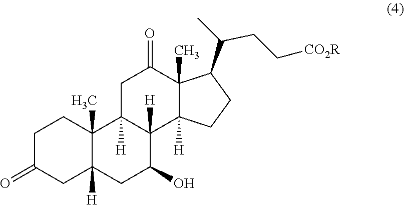

(in the presence of and with consumption of NADPH and/or NADH),
c) 3,12-diketo-7β-CA is reduced in the presence of at least one 3α-hydroxysteroid dehydrogenase (3α-HSDH) (present as isolated enzyme or expressed by a corresponding recombinant microorganism) to the corresponding 12-keto-ursodeoxycholic acid (12-keto UDCA) of formula (5)

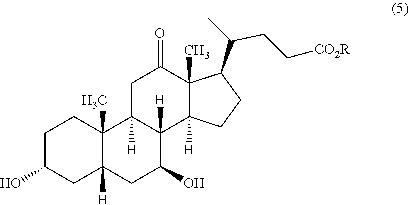

in which R has the meanings given above, (in the presence of and with consumption of NADH and/or NADPH, depending on the 3α-HSDH used) and then
d) 12-keto-UDCA of formula (5) is reduced chemically to UDCA; and
e) the reaction product is optionally further purified.
36. The process according to embodiment 35, wherein steps b) and c) are carried out in the presence of one or more recombinant microorganisms described herein, such as at least one microorganism according to embodiment 10.
37. The process according to embodiment 35 or 36, wherein steps b) and/or c) are coupled to identical or different cofactor regeneration systems (present as isolated enzyme or expressed by a corresponding recombinant microorganism).
38. The process according to embodiment 37, wherein step b) is coupled to a cofactor regeneration system, in which NADPH is regenerated by an NADP⁺-accepting FDH mutant according to the definition in one of the embodiments 20 to 24 with consumption of formic acid or a salt thereof; or is coupled to a cofactor regeneration system, in which NADPH is regenerated by an ADH with consumption of isopropanol; or is coupled to a cofactor regeneration system in which NADPH is regenerated by a GDH with consumption of glucose; or is coupled to a cofactor regeneration system in which spent NADH is regenerated by an NADH regenerating GDH, ADH or FDH.
39. The process according to embodiment 37 or 38, wherein step c) is coupled to a cofactor regeneration step, in which NADPH is regenerated by an NADP⁺-accepting FDH mutant according to the definition in one of the embodiments 20 to 24 with consumption of formic acid or a salt thereof; or wherein step c) is coupled to a cofactor regeneration step in which NADH is regenerated by an NADH-regenerating FDH mutant according to the definition in one of the embodiments 20 to 24 or by the unmutated FDH with consumption of formic acid or a salt thereof or by an NADH-regenerating GDH with consumption of glucose.
40. A process for microbial preparation of ursodeoxycholic acid (UDCA) of formula (1)

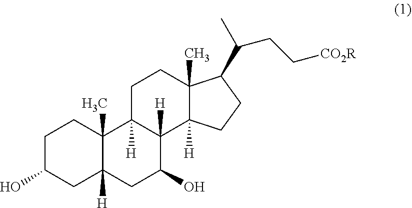

in which
R stands for alkyl, NR¹R², H, an alkali metal ion or N(R³)₄⁺, in which the residues R³ may be identical or different and stand for H or alkyl,
wherein
a) optionally a cholic acid (CA) of formula (2)

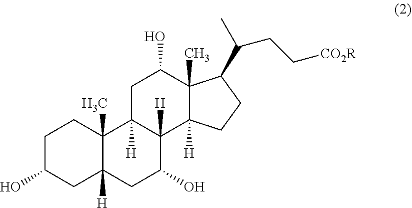

in which R has the meanings given above, is oxidized chemically to the dehydrocholic acid (DHCA) of formula (3)

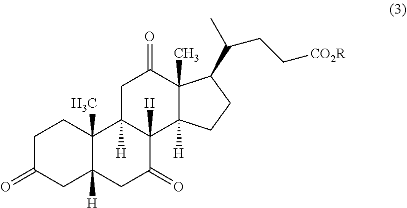

in which R has the meanings given above;
b) DHCA is reduced in the presence of at least one 7β-HSDH and in the presence of at least one 3α-HSDH to the corresponding 12-keto-ursodeoxycholic acid (12-keto UDCA) of formula (5)

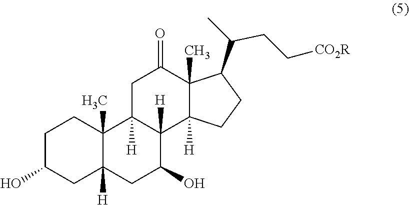

in which R has the meanings given above, (in the presence of and with consumption of NADH and/or NADPH) and then
c) 12-keto-UDCA of formula (5) is reduced chemically to UDCA; and
d) the reaction product is optionally further purified;
wherein the reactions of step b) take place microbially, i.e. in the presence of whole cells of one or more different recombinant microorganisms according to one of the embodiments 28 or 29 to 34, wherein the microorganism or microorganisms carry the enzymes necessary for the reaction and cofactor regeneration in a manner described in more detail herein, or else using at least one nucleic acid sequence according to embodiment 25.
   For example, DHCA can be reduced in the presence of at least one 7β-HSDH or mutant thereof to 3,12-diketo-7β-cholanic acid (3,12-diketo-7β-CA) of formula (4)

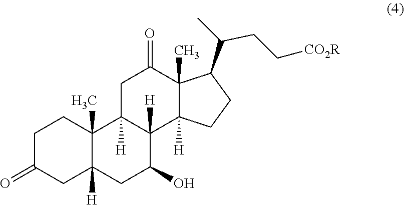

(in the presence of and with consumption of NADPH), and
3,12-diketo-7β-CA can be reduced in the presence of at least one 3α-hydroxysteroid dehydrogenase (3α-HSDH) or mutant thereof to the corresponding 12-keto-ursodeoxycholic acid (12-keto UDCA) of formula (5)

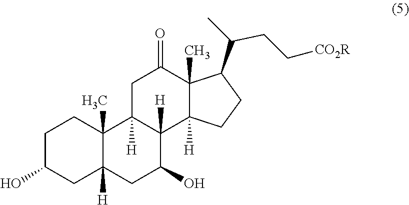

in which R has the meanings given above, (in the presence of and with consumption of NADH and/or NADPH).
   Furthermore, however, a reaction sequence is also conceivable, comprising the reduction of DHCA first with 3α-HSDH and the subsequent reduction of the resultant reaction product with 7β-HSDH, as well as both reaction sequences taking place simultaneously, on the basis of the simultaneous presence of both HSDHs.
41. The process according to embodiment 40, wherein one or more, in particular one, recombinant microorganism according to one of the embodiments 29 to 34 is used, which for example simultaneously expresses at least one 7β-HSDH mutant according to one of the embodiments 1 to 6, at least one NADP⁺-accepting FDH mutant according to one of the embodiments 20 to 24 and at least one 3α-HSDH, such as in particular according to SEQ ID NO: 6, 8 or 22 or mutants thereof in a sequence identity of at least about 60%; or which simultaneously expresses at least one 7β-HSDH mutant according to one of the embodiments 1 to 6, at least one GDH and at least one 3α-HSDH, such as in particular according to SEQ ID NO: 6, 8 or 22 or mutants thereof in a sequence identity of at least about 60%.
42. A bioreactor for carrying out a process according to one of the embodiments 35 to 41, in particular containing at least one of the enzymes (7β-HSDH, FDH, and/or 3α-HSDH or mutants thereof; or 7β-HSDH, GDH and/or 3α-HSDH or mutants thereof) or a recombinant microorganism recombinantly expressing at least one of these enzymes, especially in immobilized form.
   The present invention is not limited to the concrete embodiments described herein. Rather, a person skilled in the art will be enabled, through the teaching of the present invention, to provide further configurations of the invention without undue effort. He can, for example, also purposefully generate further enzyme mutants and screen and optimize these for the desired property profile (improved cofactor dependence and/or stability, reduced substrate inhibition); or isolate further suitable wild-type enzymes (7β- and 3α-HSDHs, FDHs, GDHs ADHs etc.) and use them according to the invention. Furthermore, for example depending on the property profile (especially cofactor dependence) of the HSDHs used, such as in particular 7β-HSDH and 3α-HSDH or mutants thereof, he can select suitable dehydrogenases usable for cofactor regeneration (GDH, FHD, ADH etc.) and mutants thereof, and distribute the selected enzymes to one or more expression constructs or vectors and therefore if necessary produce one or more recombinant microorganisms, which then make an optimized whole-cell-based method of production possible.

### Further Configurations of the Invention

### 1. General Definitions and Abbreviations Used

Unless stated otherwise, the term “7β-HSDH” denotes a dehydrogenase enzyme, which catalyzes at least the stereospecific and/or regiospecific reduction of DHCA or 7,12-diketo-3α-CA (7,12-diketo-LCA) to 3,12-diketo-7β-CA or 12-keto-UDCA in particular with stoichiometric consumption of NADPH, and optionally the corresponding reverse reaction. The enzyme can be a native or recombinantly produced enzyme. The enzyme can basically be mixed with cellular, for example protein impurities, but preferably is in pure form. Suitable methods of detection are described for example in the experimental section given below or are known from the literature (e.g. Characterization of NADP-dependent 7 beta-hydroxysteroid dehydrogenases from Peptostreptococcus productus and Eubacterium aerofaciens. S Hirano and N Masuda. Appl Environ Microbiol. 1982). Enzymes with this activity are classified under the EC number 1.1.1.201.

Unless stated otherwise, the term “3α-HSDH” denotes a dehydrogenase enzyme that catalyzes at least the stereospecific and/or regiospecific reduction of 3,12-diketo-7β-CA or DHCA to 12-keto-UDCA or 7,12-diketo-3α-CA (7,12-diketo-LCA), in particular with stoichiometric consumption of NADH and/or NADPH, and optionally the corresponding reverse reaction. Suitable methods of detection are described for example in the experimental section given below or are known from the literature. Suitable enzymes are obtainable e.g. from Comanomonas testosteroni (e.g. ATCC11996). An NADPH-dependent 3α-HSDH is known for example from rodents and can also be used. (Cloning and sequencing of the cDNA for rat liver 3 alpha-hydroxysteroid/dihydrodiol dehydrogenase, J E Pawlowski, M Huizinga and T M Penning, May 15, 1991, The Journal of Biological Chemistry, 266, 8820-8825). Enzymes with this activity are classified under EC number 1.1.1.50.

Unless stated otherwise, the term “GDH” denotes a dehydrogenase enzyme that catalyzes at least the oxidation of β-D-glucose to D-glucono-1,5-lactone with stoichiometric consumption of NAD⁺ and/or NADP⁺ and optionally the corresponding reverse reaction. Suitable enzymes are obtainable e.g. from Bacillus subtilis or Bacillus megaterium. Enzymes with this activity are classified under EC number 1.1.1.47. Unless stated otherwise, the term “FDH” denotes a dehydrogenase enzyme that catalyzes at least the oxidation of formic acid (or corresponding formate salts) to carbon dioxide with stoichiometric consumption of NAD⁺ and/or NADP⁺, and optionally the corresponding reverse reaction. Suitable methods of detection are for example described in the experimental section given below or are known from the literature. Suitable enzymes are obtainable e.g. from Candida boidinii, Pseudomonas sp, or Mycobacterium vaccae. Enzymes with this activity are classified under EC number 1.2.1.2.

A “pure form” or a “pure” or “substantially pure” enzyme is to be understood according to the invention as an enzyme with a degree of purity above 80, preferably above 90, especially above 95, and quite particularly above 99 wt %, relative to the total protein content, determined by means of usual methods of detecting proteins, for example the biuret method or protein detection according to Lowry et al. (cf. description in R. K. Scopes, Protein Purification, Springer Verlag, New York, Heidelberg, Berlin (1982)).

A “redox equivalent” means a low-molecular organic compound usable as electron donor or electron acceptor, for example nicotinamide derivatives such as NAD⁺ and NADH⁺ or their reduced forms NADH and NADPH respectively. “Redox equivalent” and “cofactor” are used as synonyms in the context of the present invention. Thus, a “cofactor” in the sense of the invention can also be described as “redox-capable cofactor”, i.e. as a cofactor that can be present in a reduced and an oxidized form.

A “spent” cofactor is to be understood as the reduced or oxidized form of the cofactor, which in the course of a specified reduction or oxidation reaction of a substrate is transformed into the corresponding oxidized or reduced form. By regeneration, the oxidized or reduced cofactor form that is formed in the reaction is converted back to the reduced or oxidized starting form, so that it is available again for the reaction of the substrate.

An “altered cofactor usage” is to be understood in the context of the present invention as a qualitative or quantitative change compared to a reference. In particular, an altered cofactor usage can be observed by undertaking amino acid sequence mutations. This change can then be determined compared to the unmutated starting enzyme. Moreover, the activity with respect to a particular cofactor can be increased or reduced by undertaking a mutation or can be prevented completely. An altered cofactor usage also comprises, however, changes such that instead of a specificity for an individual cofactor, now at least one further second cofactor, different from the first cofactor, is usable (i.e. there is an extended cofactor usage). Conversely, however, a capacity for usage of two different cofactors that was originally present can be altered so that specificity is only increased for one of these cofactors or only reduced for one of these cofactors or is completely eliminated. For example, an enzyme that is dependent on the cofactor NAD (NADH) can now, owing to a change of the cofactor usage, be dependent both on NAD (NADH) and the cofactor NADP (NADPH) or the original dependence on NAD (NADH) can be completely transformed to a dependence on NADP (NADPH) and vice versa.

The terms “NAD⁺/NADH dependence” or “NADP⁺/NADPH dependence”, unless stated otherwise, are to be interpreted widely according to the invention. These terms comprise both “specific” dependences, i.e. exclusively dependence on NAD⁺/NADH or NADP⁺/NADPH, as well as the dependence of the enzymes used according to the invention on both cofactors, i.e. dependence on NAD⁺/NADH and NADP⁺/NADPH.

This applies correspondingly to the terms “NAD⁺/NADH-accepting” or “NADP⁺/NADPH-accepting”.

The terms “NAD⁺/NADH-regenerating” or “NADP⁺/NADPH-regenerating”, unless stated otherwise, are to be interpreted widely according to the invention. These terms comprise both “specific” i.e. exclusive capacity for regenerating spent cofactor NAD⁺/NADH or NADP⁺/NADPH, and the capacity for regenerating both cofactors, i.e. NAD⁺/NADH and NADP⁺/NADPH.

“Proteinogenic” amino acids comprise in particular (single-letter code): G, A, V, L, I, F, P, M, W, S, T, C, Y, N, Q, D, E, K, R and H.

“Immobilization” means, according to the invention, the covalent or noncovalent binding of a biocatalyst used according to the invention, for example a 7β-HSDH on a solid, i.e. essentially insoluble in the surrounding liquid medium, carrier material. According to the invention, whole cells, such as the recombinant microorganisms used according to the invention, can correspondingly also be immobilized by means of such carriers.

A “substrate inhibition reduced in comparison with the unmutated enzyme” means that the substrate inhibition observed with the unmutated enzyme for a particular substrate is no longer observed, i.e. essentially is no longer measurable, or only occurs at higher substrate concentration, i.e. the Kᵢ value is increased.

“Cholic acid compound” means compounds according to the invention with the carbon skeleton structure, especially the steroid structure of cholic acid and the presence of keto and/or hydroxy or acyloxy groups in ring position 7 and optionally ring positions 3 and/or 12.

A compound of a special type, for example a “cholic acid compound” or an “ursodeoxycholic acid compound” in particular also means derivatives of the underlying starting compound (for example cholic acid or ursodeoxycholic acid).

Said derivatives comprise “salts”, for example alkali metal salts such as lithium, sodium and potassium salts of the compounds; and ammonium salts, wherein an ammonium salt comprises the NH₄⁺ salt or those ammonium salts in which at least one hydrogen atom can be replaced with a C₁-C₆-alkyl residue. Typical alkyl residues are, in particular, C₁-C₄-alkyl residues, such as methyl, ethyl, n- or i-propyl-, n-, sec- or tert-butyl, and n-pentyl and n-hexyl and the singly or multiply branched analogs thereof.

“Alkyl esters” of compounds according to the invention are, in particular, lower alkyl esters, for example C₁-C₆-alkyl esters. As nonlimiting examples, we may mention methyl, ethyl, n- or i-propyl, n-, sec- or tert-butyl esters, or longer-chain esters, for example n-pentyl and n-hexyl esters and the singly or multiply branched analogs thereof.

“Amides” are, in particular, reaction products of acids according to the invention with ammonia or primary or secondary monoamines. Such amines are for example mono- or di-C₁-C₆-alkyl monoamines, wherein the alkyl residues can optionally be further substituted independently of one another, for example with carboxyl, hydroxyl, halogen (such as F, Cl, Br, I), nitro and sulfonate groups.

“Acyl groups” according to the invention are, in particular, nonaromatic groups with 2 to 4 carbon atoms, for example acetyl, propionyl and butyryl, and aromatic groups with an optionally substituted mononuclear aromatic ring, wherein suitable substituents are selected for example from hydroxyl, halogen (such as F, Cl, Br, I), nitro and C₁-C₆-alkyl groups, for example benzoyl or toluoyl.

The hydroxysteroid compounds used or prepared according to the invention, for example cholic acid, ursodeoxycholic acid, 12-keto-chenodeoxycholic acid, chenodeoxycholic acid and 7-keto-lithocholic acid, can be used in stereoisomerically pure form or in a mixture with other stereoisomers in the process according to the invention or obtained therefrom. Preferably, however, the compounds used or prepared are used or isolated in substantially stereoisomerically pure form.

The following table gives the structural formulas, chemical names and the abbreviations used for important chemical compounds:

| Formula | Abbreviation | Chemical name |
|---|---|---|
| 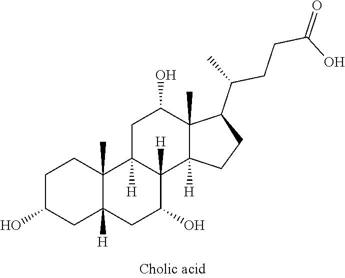 | CA | Cholic acid |
| 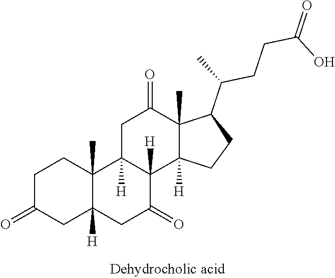 | DHCA | Dehydrocholic acid |
| 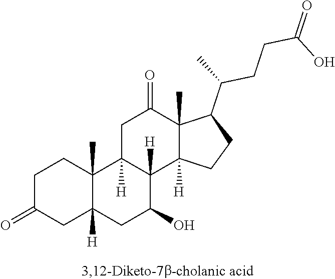 | 3,12-diketo-7β-CA | 3,12-Diketo-7β- cholanic acid |
| 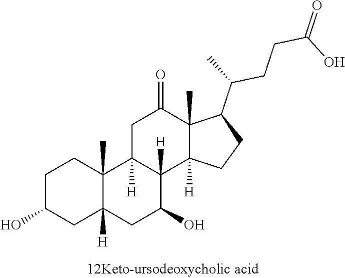 | 12Keto-UDCA | 12Keto-ursodeoxy- cholic acid |
| 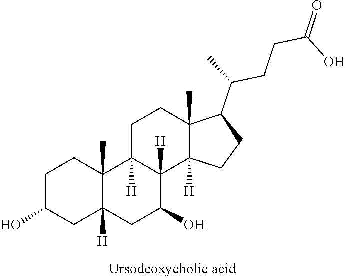 | UDCA | Ursodeoxycholic acid |
| 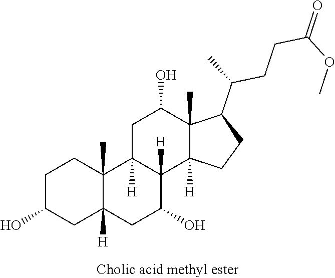 | CA methyl ester | Cholic acid methyl ester |
| 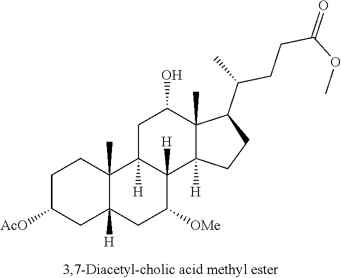 | 3,7-diacetyl-CA- methyl ester | 3,7-Diacetyl-cholic acid methyl ester* |
| 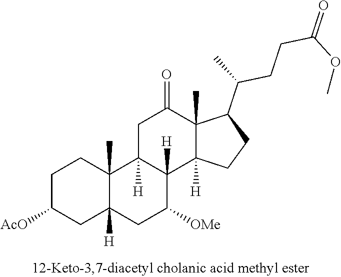 | 12-keto-3,7-diacetyl- CA methyl ester | 12-Keto-3,7-diacetyl cholanic acid methyl ester* |
| 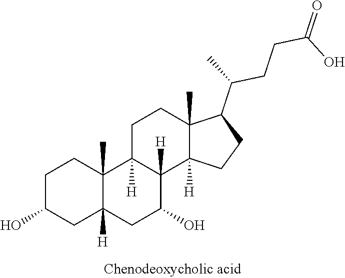 | CDCA | Chenodeoxycholic acid |
| 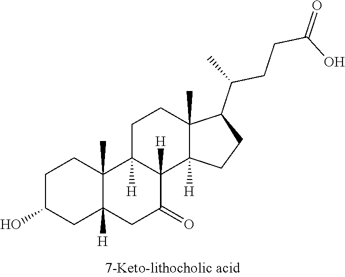 | 7-Keto-LCA | 7-Keto-lithocholic acid |
| 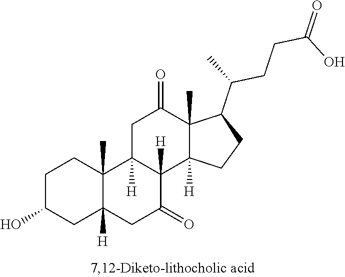 | 7,12-Diketo-LCA | 7,12-Diketo-litho- cholic acid |
| 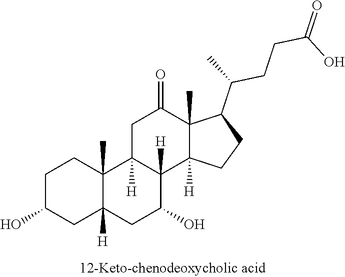 | 12-Keto-CDCA | 12-Keto-chenodeoxy- cholic acid |

### 2. Proteins

The present invention is not limited to the concretely disclosed proteins or enzymes with 7β-HSDH, FDH, GDH or 3α-HSDH activity or mutants thereof, but rather also extends to functional equivalents thereof.

“Functional equivalents” or analogs of the concretely disclosed enzymes are, in the context of the present invention, polypeptides that are different from them, but still possess the desired biological activity, for example 7β HSDH activity.

For example, “functional equivalents” are to be understood as enzymes that have, in the test used for 7β-HSDH, FDH, GDH or 3α-HSDH activity, an activity that is higher or lower by at least 1%, e.g. at least 10% or 20%, e.g. at least 50% or 75% or 90% than that of a starting enzyme, comprising an amino acid sequence defined herein.

Functional equivalents are in addition preferably stable in the pH range from 4 to 11 and advantageously possess an optimal pH in a pH range from 6 to 10, such as in particular 8.5 to 9.5, and an optimal temperature in the range from 15° C. to 80° C. or 20° C. to 70° C., for example about 45 to 60° C. or about 50 to 55° C.

The 7β-HSDH activity can be detected using various known tests. Without being restricted to this, we may mention a test using a reference substrate, e.g. CA or DHCA, under standardized conditions, as defined in the experimental section.

Tests for determining the FDH, GDH or 3α-HSDH activity are also known per se.

“Functional equivalents” according to the invention also means, in particular, “mutants”, which have in at least one sequence position of the aforementioned amino acid sequences an amino acid other than that concretely stated, but nevertheless possess one of the aforementioned biological activities. “Functional equivalents” therefore comprise the mutants obtainable by one or more, for example 1, 2, 3, 4, 5, 6, 7, 8, 9, 10, 11, 12, 13, 14 or 15, amino acid additions, substitutions, deletions and/or inversions, wherein the stated changes can occur in any sequence position, provided they lead to a mutant with the property profile according to the invention. Functional equivalence in particular also obtains when the patterns of reactivity between mutant and unaltered polypeptide coincide qualitatively, i.e. for example the same substrates are converted at different rates. Examples of suitable amino acid substitutions are presented in the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also “precursors” of the polypeptides described and “functional derivatives” and “salts” of the polypeptides.

“Precursors” are natural or synthetic precursors of the polypeptides with or without the desired biological activity.

The expression “salts” means both salts of carboxyl groups and acid addition salts of amino groups of the protein molecules according to the invention. Salts of carboxyl groups can be prepared in a manner known per se and comprise inorganic salts, for example sodium, calcium, ammonium, iron and zinc salts, and salts with organic bases, for example amines, such as triethanolamine, arginine, lysine, piperidine and the like. Acid addition salts, for example salts with mineral acids, such as hydrochloric acid or sulfuric acid and salts with organic acids, such as acetic acid and oxalic acid, are also an object of the invention.

“Functional derivatives” of polypeptides according to the invention can also be prepared on functional amino acid side groups or on their N- or C-terminal end using known techniques. Such derivatives comprise for example aliphatic esters of carboxylic acid groups, amides of carboxylic acid groups, obtainable by reaction with ammonia or with a primary or secondary amine; N-acyl derivatives of free amino groups, prepared by reaction with acyl groups; or O-acyl derivatives of free hydroxyl groups, prepared by reaction with acyl groups.

“Functional equivalents” naturally also comprise polypeptides that are obtainable from other organisms, and naturally occurring variants. For example, by sequence comparison, homologous sequence regions can be found and equivalent enzymes can be determined based on the concrete instructions of the invention.

“Functional equivalents” also comprise fragments, preferably individual domains or sequence motifs, of the polypeptides according to the invention, which for example have the desired biological function.

“Functional equivalents” are in addition fusion proteins that have one of the aforementioned polypeptide sequences or functional equivalents derived therefrom and at least one further, functionally different therefrom, heterologous sequence in functional N- or C-terminal linkage (i.e. without mutual substantial functional impairment of the fusion protein parts). Nonlimiting examples of said heterologous sequences are e.g. signal peptides, histidine anchors or enzymes.

“Functional equivalents” that are also included according to the invention are homologs of the concretely disclosed proteins. These possess at least 60%, preferably at least 75%, especially at least 85%, for example 90, 91, 92, 93, 94, 95, 96, 97, 98 or 99%, homology (or identity) to one of the concretely disclosed amino acid sequences, calculated according to the algorithm of Pearson and Lipman, Proc. Natl. Acad. Sci. (USA) 85(8), 1988, 2444-2448. A percentage homology or identity of a homologous polypeptide according to the invention means, in particular, percentage identity of the amino acid residues relative to the total length of one of the amino acid sequences concretely described herein.

The percentage identity values can also be determined on the basis of BLAST alignments, the blastp (protein-protein BLAST) algorithm, or using the Clustal settings given below.

In the case of a possible protein glycosylation, “functional equivalents” according to the invention comprise proteins of the type designated above in deglycosylated or glycosylated form and modified forms obtainable by altering the glycosylation pattern.

Homologs of the proteins or polypeptides according to the invention can be produced by mutagenesis, e.g. by point mutation, lengthening or shortening of the protein.

Homologs of the proteins according to the invention can be identified by screening combinatorial libraries of mutants, for example shortened mutants. For example, a variegated database of protein variants can be produced by combinatorial mutagenesis at the nucleic acid level, for example by enzymatic ligation of a mixture of synthetic oligonucleotides. There are a great many methods that can be used for preparing libraries of potential homologs from a degenerated oligonucleotide sequence. The chemical synthesis of a degenerated gene sequence can be carried out in an automatic DNA synthesizer, and the synthetic gene can then be ligated into a suitable expression vector. The use of a degenerated set of genes makes it possible to provide all sequences in one mixture, which encode the desired set of potential protein sequences. Methods of synthesis of degenerated oligonucleotides are known by a person skilled in the art (e.g. Narang, S. A. (1983) Tetrahedron 39: 3; Itakura et al. (1984) Annu. Rev. Biochem. 53: 323; Itakura et al., (1984) Science 198: 1056; Ike et al. (1983) Nucleic Acids Res. 11: 477).

Several techniques are known in the prior art for screening gene products of combinatorial libraries, that have been produced by point mutations or shortening, and for screening cDNA libraries for gene products with a selected property. These techniques can be adapted to the rapid screening of gene banks that have been produced by combinatorial mutagenesis of homologs according to the invention. The techniques used most often for screening large gene banks, which are based on a high-throughput analysis, comprise cloning the gene bank into replicatable expression vectors, transforming suitable cells with the resultant vector bank and expressing the combinatorial genes under conditions in which detection of the desired activity facilitates the isolation of the vector that encodes the gene whose product was detected. Recursive ensemble mutagenesis (REM), a technique that increases the frequency of functional mutants in the banks, can be used in combination with the screening tests, in order to identify homologs (Arkin and Yourvan (1992) PNAS 89: 7811-7815; Delgrave et al. (1993) Protein Engineering 6(3): 327-331).

The invention further comprises the use of the 7β-HSDH wild type from Collinsella aerofaciens ATCC 25986, as described in the applicant's earlier international patent application PCT/EP2010/068576, which is expressly referred to hereby.

This 7β-HSDH obtainable from Collinsella aerofaciens DSM 3979 is in particular characterized by at least one other of the following properties, for example 2, 3, 4, 5, 6 or 7 or all such properties:
a) molecular weight (SDS-gel electrophoresis): about 28-32 kDa, especially about 29 to 31 kDa or about 30 kDa;
b) molecular weight (gel filtration, in nondenaturing conditions, such as in particular without SDS): about 53 to 60 kDa, especially about 55 to 57 kDa, such as 56.1 kDa. This proves the dimeric nature of the 7β-HSDH from Collinsella aerofaciens DSM 3979;
c) stereoselective reduction of the 7-carbonyl group of 7-keto-LCA to a 7β-hydroxyl group;
d) optimal pH for the oxidation of UDCA in the range from pH 8.5 to 10.5, especially 9 to 10;
e) optimal pH for the reduction of DHCA and 7-keto-LCA in the range from pH 3.5 to 6.5, especially at pH 4 to 6;
f) at least one kinetic parameter from the following table for at least one of the substrates/cofactors mentioned there; in the range of ±20%, especially ±10%, ±5%, ±3%, ±2% or ±1% around the value stated concretely in each case in the following table.

| | K_{M} | Vₘₐₓ | k_{cat} |
|---|---|---|---|
| | (μM) | (U/mg protein)^{b)} | (1 μmol/(μmol × min)) |
| NADP⁺ | 5.32 | 30.58 | 944.95 |
| NADPH | 4.50 | 33.44 | 1033.44 |
| UDCA | 6.23 | 38.17 | 1179.39 |
| 7-Keto-LCA | 5.20 | 30.77 | 950.77 |
| DHCA | 9.23 | 28.33 | 875.35 |
| NAD⁺ | —^{a)} | — | Traces |
| NADH | — | — | Traces |

^{a)}could not be determined, owing to the very low activity
^{b)}1 U = 1 μmol/min

g) phylogenetic sequence similarity of the prokaryotic 7β-HSDH from Collinsella aerofaciens DSM 3979, related to the animal 11β-HSDH subgroup, comprising Cavia porcellus, Homo sapiens and Mus musculus.

For example, this 7β-HSDH shows the following properties or combinations of properties: a); b); a) and b); a) and/or b) and c); a) and/or b) and c) and d); a) and/or b) and c) and d) and e); a) and/or b) and c) and d) and e) and f).

A 7β-HSDH of this kind or a functional equivalent derived therefrom is moreover characterized by
a) the stereospecific reduction of a 7-ketosteroid to the corresponding 7β-hydroxysteroid, and/or
b) the regiospecific hydroxylation of a ketosteroid comprising a keto group in 7-position and at least one further keto group on the steroid skeleton to the corresponding 7β-hydroxysteroid, such as in particular catalyzed by dehydrocholic acid (DHCA) in 7-position to the corresponding 3,12-diketo-7β-cholanic acid, and e.g. is NADPH-dependent.

Said 7β-HSDH has in particular an amino acid sequence according to SEQ ID NO:2 (accession No.: ZP_01773061) or a sequence derived therefrom with a degree of identity of at least 60%, e.g. at least 65, 70, 75, 80, 85, or 90, e.g. at least 91, 92, 93, 94, 95, 96, 97, 98, 99 or 99.5% to this sequence; optionally additionally characterized by one of the following properties or combinations of properties: a); b); a) and b); a) and/or b) and c); a) and/or b) and c) and d); a) and/or b) and c) and d) and e); a) and/or b) and c) and d) and e) and f) according to the above definition.

### 3. Nucleic Acids and Constructs

### 3.1 Nucleic Acids

The invention also relates to nucleic acid sequences that code for an enzyme with 7β-HSDH, FDH, GDH and/or 3α-HSDH activity and the mutants thereof.

The present invention also relates to nucleic acids with a specified degree of identity to the concrete sequences described herein.

“Identity” between two nucleic acids means the identity of the nucleotides over the total nucleic acid length in each case, especially the identity that is calculated by comparison by means of the Vector NTI Suite 7.1 software of the company Informax (USA) employing the Clustal method (Higgins D G, Sharp P M. Fast and sensitive multiple sequence alignments on a microcomputer. Comput Appl. Biosci. 1989 April; 5(2): 151-1) setting the following parameters:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighting | 0 |
| Pairwise alignment parameters: | |
| FAST algorithm | on |
| K-tuple size | 1 |
| Gap penalty | 3 |
| Window size | 5 |
| Number of best diagonals | 5 |

As an alternative, the identity can also be determined according to Chenna, Ramu, Sugawara, Hideaki, Koike, Tadashi, Lopez, Rodrigo, Gibson, Toby J, Higgins, Desmond G, Thompson, Julie D. Multiple sequence alignment with the Clustal series of programs. (2003) Nucleic Acids Res 31 (13): 3497-500, and with the following parameters:

| | |
|---|---|
| DNA gap open penalty | 15.0 |
| DNA gap extension penalty | 6.66 |
| DNA matrix | Identity |
| Protein gap open penalty | 10.0 |
| D Protein gap extension penalty | 0.2 |
| Protein matrix | Gonnet |
| Protein/DNA ENDGAP | −1 |
| Protein/DNA GAPDIST | 4 |

All nucleic acid sequences mentioned herein (single- and double-stranded DNA and RNA sequences, for example cDNA and mRNA) can be produced in a manner known per se by chemical synthesis from the nucleotide building blocks, for example by fragment condensation of individual overlapping, complementary nucleic acid building blocks of the double helix. The chemical synthesis of oligonucleotides can be carried out for example in a known manner by the phosphoroamidite method (Voet, Voet, 2nd edition, Wiley Press New York, pages 896-897). The adding on of synthetic oligonucleotides and filling of gaps using the Klenow fragment of DNA polymerase and ligation reactions and general cloning techniques are described in Sambrook et al. (1989), Molecular Cloning: A Laboratory Manual, Cold Spring Harbor Laboratory Press.

The invention also relates to nucleic acid sequences (single- and double-stranded DNA and RNA sequences, for example cDNA and mRNA) coding for one of the above polypeptides and functional equivalents thereof, which are accessible for example using artificial nucleotide analogs.

The invention relates both to isolated nucleic acid molecules, which code for polypeptides or proteins according to the invention or biologically active segments thereof, and to nucleic acid fragments that can be used e.g. as hybridization probes or primers for identification or amplification of coding nucleic acids according to the invention.

The nucleic acid molecules according to the invention can moreover contain untranslated sequences from the 3′- and/or 5′-end of the coding gene region.

The invention further comprises the nucleic acid molecules complementary to the concretely described nucleotide sequences, or a segment thereof.

The nucleotide sequences according to the invention make it possible to produce probes and primers that can be used for identification and/or cloning of homologous sequences in other cell types and organisms. These probes or primers usually comprise a nucleotide sequence region, which hybridizes under “stringent” conditions (see below) to at least about 12, preferably at least about 25, for example about 40, 50 or 75 successive nucleotides of a sense strand of a nucleic acid sequence according to the invention or a corresponding antisense strand.

An “isolated” nucleic acid molecule is separated from other nucleic acid molecules that are present in the natural source of the nucleic acid and can moreover be essentially free from other cellular material or culture medium, when it is produced by recombinant techniques, or free from chemical precursors or other chemicals, when it is synthesized chemically.

A nucleic acid molecule according to the invention can be isolated using standard techniques of molecular biology and the sequence information provided according to the invention. For example, cDNA can be isolated from a suitable cDNA library, using one of the concretely disclosed complete sequences or a segment thereof as hybridization probe and standard hybridization techniques (as described for example in Sambrook, J., Fritsch, E. F. and Maniatis, T. Molecular Cloning: A Laboratory Manual. 2nd ed., Cold Spring Harbor Laboratory, Cold Spring Harbor Laboratory Press, Cold Spring Harbor, N Y, 1989). Moreover, a nucleic acid molecule, comprising one of the disclosed sequences or a segment thereof, can be isolated by polymerase chain reaction, using the oligonucleotide primers that are prepared on the basis of this sequence. The nucleic acid thus amplified can be cloned into a suitable vector and can be characterized by DNA sequence analysis. The oligonucleotides according to the invention can also be produced by standard synthesis techniques, e.g. with an automatic DNA synthesizer.

Nucleic acid sequences according to the invention or derivatives thereof, homologs or parts of these sequences can be isolated for example with usual hybridization methods or PCR technology from other bacteria, e.g. via genomic or cDNA libraries. These DNA sequences hybridize under standard conditions to the sequences according to the invention.

“Hybridize” means the capacity of a polynucleotide or oligonucleotide for binding to an almost complementary sequence under standard conditions, whereas under these conditions nonspecific bindings do not occur between noncomplementary partners. For this, the sequences can be complementary to 90-100%. The property of complementary sequences of being able to bind specifically to one another is utilized for example in Northern or Southern blotting or in primer binding in PCR or RT-PCR.

Advantageously, short oligonucleotides of the conserved regions are used for hybridization. It is also possible, however, to use longer fragments of the nucleic acids according to the invention or the complete sequences for hybridization. These standard conditions vary depending on the nucleic acid used (oligonucleotide, longer fragment or complete sequence) or depending on which type of nucleic acid, DNA or RNA, is used for the hybridization. Thus, for example, the melting points for DNA:DNA hybrids are approx. 10° C. lower than those of DNA:RNA hybrids of the same length.

Standard conditions mean for example, depending on the nucleic acid, temperatures between 42 and 58° C. in an aqueous buffer solution with a concentration between 0.1 and 5×SSC (1×SSC=0.15 M NaCl, 15 mM sodium citrate, pH 7.2) or additionally in the presence of 50% formamide such as for example 42° C. in 5×SSC, 50% formamide. Advantageously the hybridization conditions for DNA:DNA hybrids are 0.1×SSC and temperatures between about 20° C. and 45° C., preferably between about 30° C. and 45° C. For DNA:RNA hybrids the hybridization conditions are advantageously 0.1×SSC and temperatures between about 30° C. and 55° C., preferably between about 45° C. and 55° C. These temperatures stated for the hybridization are for example calculated melting point values for a nucleic acid with a length of approx. 100 nucleotides and a G+C content of 50% in the absence of formamide. The experimental conditions for DNA hybridization are described in relevant genetics textbooks, for example Sambrook et al., “Molecular Cloning”, Cold Spring Harbor Laboratory, 1989, and can be calculated from formulas known by a person skilled in the art for example depending on the length of the nucleic acids, the type of hybrids or the G+C content. A person skilled in the art can find further information on hybridization from the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

“Hybridization” can in particular take place under stringent conditions. These hybridization conditions are described for example in Sambrook, J., Fritsch, E. F., Maniatis, T., in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6.

“Stringent” hybridization conditions are understood in particular as: incubation at 42° C. overnight in a solution consisting of 50% formamide, 5×SSC (750 mM NaCl, 75 mM trisodium citrate), 50 mM sodium phosphate (pH7.6), 5×Denhardt solution, 10% dextran sulfate and 20 g/ml denatured, sheared salmon sperm DNA, followed by a filter washing step with 0.1×SSC at 65° C.

The invention also relates to derivatives of the concretely disclosed or derivable nucleic acid sequences.

Thus, further nucleic acid sequences according to the invention can be derived e.g. from SEQ ID NO:1, 5, 7, 14, 19, 34 or 47 and differ from them by addition, substitution, insertion or deletion of individual or several nucleotides, but furthermore code for polypeptides with the desired property profile.

The invention also covers those nucleic acid sequences that comprise so-called silent mutations or are altered corresponding to the codon usage of a special original or host organism, compared to a concretely stated sequence, as well as naturally occurring variants, for example splice variants or allele variants, thereof.

The invention also relates to sequences obtainable by conservative nucleotide substitutions (i.e. the amino acid in question is replaced with an amino acid of the same charge, size, polarity and/or solubility).

The invention also relates to the molecules derived by sequence polymorphisms from the concretely disclosed nucleic acids. These genetic polymorphisms can exist between individuals within a population owing to natural variation. These natural variations usually bring about a variance from 1 to 5% in the nucleotide sequence of a gene.

Derivatives of the nucleic acid sequence according to the invention with the sequence SEQ ID NO:1, 5, 7, 14, 19, 34 or 47 mean for example allele variants that have at least 60% homology at the derived amino acid level, preferably at least 80% homology, quite especially preferably at least 90% homology over the total sequence region (regarding homology at the amino acid level, reference may be made to the above information for the polypeptides). The homologies can advantageously be higher on partial regions of the sequences.

Furthermore, derivatives are also to be understood as homologs of the nucleic acid sequences according to the invention, especially of SEQ ID NO:1, 5, 7, 14, 19, 34 or 47, for example fungal or bacterial homologs, shortened sequences, single-stranded DNA or RNA of the coding and noncoding DNA sequence. For example, homologs to SEQ ID NO:1, 5, 7, 14, 19, 34 or 47 possess, at DNA level, a homology of at least 40%, preferably of at least 60%, especially preferably of at least 70%, quite especially preferably of at least 80% over the whole DNA region given in SEQ ID NO: 1, 5, 7, 14, 19, 34 or 47.

In addition, derivatives are to be understood for example as fusions with promoters. The promoters that precede the stated nucleotide sequences can be altered by at least one nucleotide exchange, at least one insertion, inversion and/or deletion, but without the functionality or effectiveness of the promoters being impaired. Moreover, the effectiveness of the promoters can be increased by altering their sequence or they can be exchanged completely with more effective promoters even of organisms of a different species.

Furthermore, methods for producing functional mutants are known by a person skilled in the art.

Depending on the technology used, a person skilled in the art can insert completely random or even more targeted mutations in genes or also noncoding nucleic acid regions (which are for example important for the regulation of expression) and then prepare gene banks. The methods of molecular biology required for this are known by a person skilled in the art and for example are described in Sambrook and Russell, Molecular Cloning. 3rd edition, Cold Spring Harbor Laboratory Press 2001.

Methods for modifying genes and therefore for modifying the protein that these encode have long been familiar to a person skilled in the art, such as for example
site-directed mutagenesis, giving targeted exchange of individual or several nucleotides of a gene (Trower M K (Publ.) 1996; In vitro mutagenesis protocols. Humana Press, New Jersey),
saturation mutagenesis, in which a codon for any amino acid can be exchanged or added at any site of a gene (Kegler-Ebo D M, Docktor C M, DiMaio D (1994) Nucleic Acids Res 22: 1593; Barettino D, Feigenbutz M, Valcarel R, Stunnenberg H G (1994) Nucleic Acids Res 22: 541; Barik S (1995) Mol Biotechnol 3: 1),
the error-prone polymerase chain reaction (error-prone PCR), in which nucleotide sequences are mutated by incorrectly functioning DNA polymerases (Eckert K A, Kunkel T A (1990) Nucleic Acids Res 18: 3739);
the passaging of genes in mutator strains, in which, for example owing to defective DNA-repair mechanisms, there is an increased mutation rate of nucleotide sequences (Greener A, Callahan M, Jerpseth B (1996) An efficient random mutagenesis technique using an E. coli mutator strain. In: Trower M K (Publ.) In vitro mutagenesis protocols. Humana Press, New Jersey), or
DNA shuffling, in which a pool of closely related genes is formed and digested and the fragments are used as template for a polymerase chain reaction, in which through repeated strand separation and bringing together again, finally mosaic genes of full length are produced (Stemmer W P C (1994) Nature 370: 389; Stemmer W P C (1994) Proc Natl Acad Sci USA 91: 10747).

Using so-called directed evolution (described inter alia in Reetz M T and Jaeger K-E (1999), Topics Curr Chem 200: 31; Zhao H, Moore J C, Volkov A A, Arnold F H (1999), Methods for optimizing industrial enzymes by directed evolution, In: Demain A L, Davies J E (Publ.) Manual of industrial microbiology and biotechnology. American Society for Microbiology), a person skilled in the art can produce functional mutants in a targeted manner and on a large scale. In a first step, firstly gene banks of the respective proteins are produced, for example using the methods given above. The gene banks are expressed in a suitable manner, for example by bacteria or by phage-display systems.

The relevant genes of host organisms that express functional mutants with properties that largely correspond to the desired properties can be submitted to another round of mutation. The steps of mutation and of selection or screening can be repeated iteratively until the functional mutants present have the desired properties to a sufficient degree. With this iterative procedure, a limited number of mutations, for example 1 to 5 mutations, can be performed in steps and assessed and selected for their influence on the relevant enzyme property. The selected mutant can then be submitted to another mutation step in the same way. As a result, the number of individual mutants to be investigated can be reduced significantly.

The results according to the invention provide important information regarding structure and sequence of the enzymes in question, which is necessary for targeted generation of further enzymes with desired modified properties. In particular, so-called “hot spots” can be defined, i.e. sequence segments that are potentially suitable for modifying an enzyme property by introducing targeted mutations.

### 3.2 Constructs

The invention further relates to expression constructs containing, under the genetic control of regulatory nucleic acid sequences, a nucleic acid sequence coding for at least one polypeptide according to the invention; and vectors, comprising at least one of these expression constructs.

“Expression unit” means, according to the invention, a nucleic acid with expression activity, which comprises a promoter, as defined herein, and, after functional linking with a nucleic acid to be expressed or a gene, regulates the expression, i.e. the transcription and the translation of this nucleic acid or of this gene. Therefore the term “regulatory nucleic acid sequence” is also used in this context. In addition to the promoter, other regulatory elements, for example enhancers, can be present.

“Expression cassette” or “expression construct” means, according to the invention, an expression unit that is functionally linked to the nucleic acid to be expressed or the gene to be expressed. In contrast to an expression unit, an expression cassette therefore comprises not only nucleic acid sequences that regulate transcription and translation, but also the nucleic acid sequences that should be expressed as protein as a result of the transcription and translation.

The terms “expression” or “overexpression” describe, in the context of the invention, the production or increase in the intracellular activity of one or more enzymes in a microorganism, which are encoded by the corresponding DNA. For this, for example a gene can be inserted in an organism, a gene that is present can be replaced with another gene, the copy number of the gene or genes can be increased, a strong promoter can be used or a gene can be used that codes for a corresponding enzyme with a high activity, and these measures can optionally be combined.

Preferably said constructs according to the invention comprise a promoter 5′-upstream of the respective coding sequence and 3′-downstream a terminator sequence and optionally further usual regulatory elements, in each case operatively linked with the coding sequence.

“Promoter”, a “nucleic acid with promoter activity” or a “promoter sequence” mean, according to the invention, a nucleic acid, which in functional linkage with a nucleic acid to be transcribed, regulates the transcription of said nucleic acid.

“Functional” or “operational” linkage means in this context for example the sequential arrangement of one of the nucleic acids with promoter activity and a nucleic acid sequence to be transcribed and optionally further regulatory elements, for example nucleic acid sequences that ensure the transcription of nucleic acids, and for example a terminator, in such a way that each of the regulatory elements can fulfill its function in the transcription of the nucleic acid sequence. This does not necessarily require a direct linkage in the chemical sense. Genetic control sequences, for example enhancer sequences, can exert their function on the target sequence even from more remote positions or even from other DNA molecules. Arrangements are preferred in which the nucleic acid sequence to be transcribed is positioned behind the promoter sequence (i.e. at the 3′-end), so that the two sequences are linked together covalently. The distance between the promoter sequence and the nucleic acid sequence that is to undergo transgene expression can be less than 200 base pairs, or less than 100 base pairs or less than 50 base pairs.

In addition to promoters and terminators, examples of other regulatory elements that may be mentioned are targeting sequences, enhancers, polyadenylation signals, selectable markers, amplification signals, replication origins and the like. Suitable regulatory sequences are described for example in Goeddel, Gene Expression Technology: Methods in Enzymology 185, Academic Press, San Diego, CA (1990).

Nucleic acid constructs according to the invention comprise, in particular, sequence SEQ ID NO:1, 5, 7, 14, 19, 34 or 47 or derivatives and homologs thereof, and the nucleic acid sequences that can be derived therefrom, which can advantageously be linked operationally or functionally with one or more regulatory signals for controlling, e.g. increasing, gene expression.

In addition to these regulatory sequences, the natural regulation of these sequences can still be present before the actual structural genes and optionally can have been genetically modified, so that the natural regulation is switched off and expression of the genes is increased. However, the nucleic acid construct can also be of simpler construction, i.e. no additional regulatory signals have been inserted before the coding sequence and the natural promoter with its regulation has not been removed. Instead, the natural regulatory sequence is mutated so that regulation no longer occurs and gene expression is increased.

A preferred nucleic acid construct advantageously also contains one or more of the aforementioned “enhancer” sequences, functionally linked to the promoter, which make increased expression of the nucleic acid sequence possible. Additional advantageous sequences can also be inserted at the 3′-end of the DNA sequences, such as further regulatory elements or terminators. The construct can contain one or more copies of the nucleic acids according to the invention. The construct can also contain further markers, such as antibiotic resistances or auxotrophic complementation genes, optionally for selection of the construct.

Examples of suitable regulatory sequences are contained in promoters such as cos, tac, trp, tet, trp-tet, Ipp, lac, Ipp-lac, lacI^{q-}, T7, T5, T3, gal, trc, ara, rhaP (rhaP_{BAD}) SP6, lambda-P_{R} or in the lambda-P_{L} promoter, which advantageously find application in Gram-negative bacteria. Further advantageous regulatory sequences are contained for example in the Gram-positive promoters amy and SPO2, in the yeast or fungus promoters ADC1, MFalpha, AC, P-60, CYC1, GAPDH, TEF, rp28, ADH. Artificial promoters can also be used for regulation.

For expression in a host organism, the nucleic acid construct is advantageously inserted into a vector, for example a plasmid or a phage, which makes optimal expression of the genes in the host possible. Apart from plasmids and phages, vectors are also to be understood as all other vectors known by a person skilled in the art, e.g. viruses, such as SV40, CMV, baculovirus and adenovirus, transposons, IS elements, phasmids, cosmids, and linear or circular DNA. These vectors can be replicated autonomously in the host organism or can be replicated chromosomally. These vectors represent another configuration of the invention.

Suitable plasmids are for example pLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-B1, λgt11 or pBdCI in E. coli, pIJ101, pIJ364, pIJ702 or pIJ361 in Streptomyces, pUB110, pC194 or pBD214 in Bacillus, pSA77 or pAJ667 in Corynebacterium, pALS1, pIL2 or pBB116 in fungi, 2alphaM, pAG-1, YEp6, YEp13 or pEMBLYe23 in yeasts or pLGV23, pGHlac⁺, pBIN19, pAK2004 or pDH51 in plants. The aforementioned plasmids represent a small selection of the possible plasmids. Further plasmids are well known by a person skilled in the art and can for example be found in the book Cloning Vectors (eds. Pouwels P. H. et al. Elsevier, Amsterdam-New York-Oxford, 1985, ISBN 0 444 904018).

In another configuration of the vector, the vector containing the nucleic acid construct according to the invention or the nucleic acid according to the invention can also advantageously be inserted in the form of a linear DNA into the microorganisms and can be integrated by heterologous or homologous recombination into the genome of the host organism. This linear DNA can consist of a linearized vector such as a plasmid or only of the nucleic acid construct or the nucleic acid according to the invention.

For optimal expression of heterologous genes in organisms, it is advantageous to modify the nucleic acid sequences according to the specific “codon usage” used in the organism. The “codon usage” can easily be determined on the basis of computer evaluations of other known genes of the organism in question.

An expression cassette according to the invention is prepared by fusion of a suitable promoter with a suitable coding nucleotide sequence and a terminator or polyadenylation signal. For this, usual recombination and cloning techniques are used, such as are described for example in T. Maniatis, E. F. Fritsch and J. Sambrook, Molecular Cloning: A Laboratory Manual, Cold Spring Harbor Laboratory, Cold Spring Harbor, NY (1989) and in T. J. Silhavy, M. L. Berman and L. W. Enquist, Experiments with Gene Fusions, Cold Spring Harbor Laboratory, Cold Spring Harbor, NY (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector, which makes optimal expression of the genes in the host possible. Vectors are well known by a person skilled in the art and can be found for example in “Cloning Vectors” (Pouwels P. H. et al., Publ., Elsevier, Amsterdam-New York-Oxford, 1985).

### 4. Microorganisms

Depending on context, the term “microorganism” means the starting (wild-type) microorganism or a genetically modified, recombinant microorganism, or both.

Using the vectors according to the invention, recombinant microorganisms can be produced, which for example have been transformed with at least one vector according to the invention and can be used for producing the polypeptides according to the invention. Advantageously, the recombinant constructs according to the invention described above are introduced into a suitable host system and expressed. Preferably, common cloning and transfection methods known by a person skilled in the art are used, for example coprecipitation, protoplast fusion, electroporation, retroviral transfection and the like, to bring about expression of the stated nucleic acids in the respective expression system. Suitable systems are described for example in Current Protocols in Molecular Biology, F. Ausubel et al., Publ., Wiley Interscience, New York 1997, or Sambrook et al. Molecular Cloning: A Laboratory Manual. 2nd ed., Cold Spring Harbor Laboratory, Cold Spring Harbor Laboratory Press, Cold Spring Harbor, N Y, 1989. A review of bacterial expression systems for the heterologous expression of proteins is also provided for example by Terpe, K. Appl. Microbiol. Biotechnol. (2006) 72: 211-222.

In principle, all prokaryotic or eukaryotic organisms may come into consideration as recombinant host organisms for the nucleic acid according to the invention or the nucleic acid construct. Advantageously, microorganisms such as bacteria, fungi or yeasts are used as host organisms. Advantageously, Gram-positive or Gram-negative bacteria are used, preferably bacteria of the families Enterobacteriaceae, Pseudomonadaceae, Rhizobiaceae, Streptomycetaceae or Nocardiaceae, especially preferably bacteria of the genera Escherichia, Pseudomonas, Streptomyces, Nocardia, Burkholderia, Salmonella, Agrobacterium, Clostridium or Rhodococcus. The genus and species Escherichia coli is quite especially preferred. Further advantageous bacteria can be found, moreover, in the group of alpha-proteobacteria, beta-proteobacteria or gamma-proteobacteria.

The host organism or the host organisms according to the invention preferably contain at least one of the nucleic acid sequences, nucleic acid constructs or vectors described in this invention, which code for an enzyme with 7β-HSDH activity according to the above definition.

The organisms used in the process according to the invention are grown or cultured in a manner known by a person skilled in the art, depending on the host organism. Microorganisms are as a rule grown in a liquid medium, which contains a carbon source generally in the form of sugars, a nitrogen source generally in the form of organic nitrogen sources such as yeast extract or salts such as ammonium sulfate, trace elements such as iron, manganese, magnesium salts and optionally vitamins, at temperatures between 0° C. and 100° C., preferably between 10° C. and 60° C. with oxygen aeration. The pH of the liquid nutrient medium can be maintained at a fixed value, i.e. during growing it may or may not be regulated. Culture can be batchwise, semi-batchwise or continuous. Nutrients can be supplied at the start of fermentation or can be replenished semi-continuously or continuously.

### 5. Preparation of UDCA

Step 1: Chemical Reaction from CA to DHCA

The hydroxy groups of CA are oxidized with chromic acid or chromates in acidic solution (e.g. H₂SO₄) to carbonyl groups in a manner known per se by the classical chemical route. DHCA is formed.

### Step 2: Enzymatic or Microbial Conversion of DHCA to 12-Keto-UDCA

In aqueous solution, DHCA is reduced by 3α-HSDH and 7β-HSDH or mutants thereof specifically to 12-keto-UDCA in the presence of NADPH or NADH. The cofactor NADPH or NADH can be regenerated by an ADH or FDH or GDH or mutants thereof from isopropanol or sodium formate or glucose. The reaction goes under mild conditions. For example, the reaction can be carried out at pH=6 to 9, especially about pH=8 and at about 10 to 30, 15 to 25 or about 23° C. In the case of a microbial reaction step, recombinant microorganisms that express the necessary enzyme activity/activities can be cultured in the presence of the substrate to be converted (DHCA) anaerobically or aerobically in suitable liquid media. Suitable cultivation conditions are known per se by a person skilled in the art. They comprise reactions in the pH range of for example 5 to 10 or 6 to 9, at temperatures in the range from 10 to 60 or 15 to 45 or 25 to 40 or 37° C. Suitable media comprise for example the LB and TB media described below. The reaction can take place for example batchwise or continuously or in other usual process variants (as described above). The reaction time can for example range from minutes to several hours or days, and can be e.g. 1 h to 48 h. Optionally, if enzyme activity is not expressed continuously, this can be induced by adding a suitable inductor, after reaching a target cell density, e.g. of about OD₆₀₀=0.5 to 1.0.

Further possible suitable modifications of the microbial production process with respect to fermentation mode, additions to the medium, enzyme immobilization and isolation of the valuable substances can also be found in the following section concerning “Production of the enzymes or mutants”.

### Step 3: Chemical Conversion of 12-Keto-UDCA to UDCA

The 12-carbonyl group of 12-keto-UDCA is removed by means of Wolff-Kishner reduction in a manner known per se, with formation of UDCA from 12-keto-UDCA. In the reaction, first the carbonyl group is reacted with hydrazine to hydrazone. Then the hydrazone is heated in the presence of a base (e.g. KOH) to 200° C., with cleavage of nitrogen and formation of UDCA.

### 6. Recombinant Production of the Enzymes and Mutants

The invention further relates to processes for recombinant production of polypeptides according to the invention or functional, biologically active fragments thereof, wherein a polypeptide-producing microorganism is cultured, optionally expression of the polypeptides is induced and the latter are isolated from the culture. The polypeptides can also be produced on an industrial scale in this way, if this is desirable.

The microorganisms produced according to the invention can be cultured continuously or discontinuously in the batch method (batch culture) or in the fed batch or repeated fed batch method. A summary of known cultivation methods can be found in Chmiel's textbook (Bioprozeßtechnik 1. Einführung in die Bioverfahrenstechnik [Bioprocess technology 1. Introduction to bioprocess engineering] (Gustav Fischer Verlag, Stuttgart, 1991)) or in the textbook by Storhas (Bioreaktoren und periphere Einrichtungen [Bioreactors and peripheral equipment]) (Vieweg Verlag, Brunswick/Wiesbaden, 1994)).

The culture medium to be used must suitably fulfill the requirements of the respective strains. Descriptions of culture media for various microorganisms are given in the manual “Manual of Methods for General Bacteriology” of the American Society for Bacteriology (Washington D.C., USA, 1981).

These media usable according to the invention usually comprise one or more carbon sources, nitrogen sources, inorganic salts, vitamins and/or trace elements.

Preferred carbon sources are sugars, such as mono-, di- or polysaccharides. Very good carbon sources are for example glucose, fructose, mannose, galactose, ribose, sorbose, ribulose, lactose, maltose, sucrose, raffinose, starch or cellulose. Sugars can also be added to the media via complex compounds, such as molasses, or other by-products of sugar refining. It may also be advantageous to add mixtures of various carbon sources. Other possible carbon sources are oils and fats such as soybean oil, sunflower oil, peanut oil and coconut oil, fatty acids such as palmitic acid, stearic acid or linoleic acid, alcohols such as glycerol, methanol or ethanol and organic acids such as acetic acid or lactic acid.

Nitrogen sources are usually organic or inorganic nitrogen compounds or materials that contain these compounds. Examples of nitrogen sources comprise ammonia gas or ammonium salts, such as ammonium sulfate, ammonium chloride, ammonium phosphate, ammonium carbonate or ammonium nitrate, nitrates, urea, amino acids or complex nitrogen sources, such as corn-steep liquor, soybean flour, soybean protein, yeast extract, meat extract and others. The nitrogen sources can be used individually or as a mixture.

Inorganic salt compounds that can be contained in the media comprise the chloride, phosphorus or sulfate salts of calcium, magnesium, sodium, cobalt, molybdenum, potassium, manganese, zinc, copper and iron.

The sulfur source used can be inorganic sulfur compounds such as sulfates, sulfites, dithionites, tetrathionates, thiosulfates, sulfides but also organic sulfur compounds, such as mercaptans and thiols.

The phosphorus source used can be phosphoric acid, potassium dihydrogen phosphate or dipotassium hydrogen phosphate or the corresponding sodium-containing salts.

Chelating agents can be added to the medium in order to keep the metal ions in solution. Especially suitable chelating agents comprise dihydroxyphenols, such as catechol or protocatechuate, or organic acids, such as citric acid.

The fermentation media used according to the invention usually also contain other growth factors, such as vitamins or growth promoters, which include for example biotin, riboflavin, thiamine, folic acid, nicotinic acid, panthothenate and pyridoxine. Growth factors and salts are often derived from complex components of the media, such as yeast extract, molasses, corn-steep liquor and the like. Moreover, suitable precursors can be added to the culture medium. The exact composition of compounds in the medium is strongly dependent on the particular experiment and is decided individually for each specific case. Information on media optimization can be found in the textbook “Applied Microbiol. Physiology, A Practical Approach” (Publ. P. M. Rhodes, P. F. Stanbury, IRL Press (1997) p. 53-73, ISBN 0 19 963577 3). Growth media can also be obtained from commercial suppliers, such as Standard 1 (Merck) or BHI (brain heart infusion, DIFCO) and the like.

All components of the media are sterilized, either with heat (20 min at 1.5 bar and 121° C.) or by sterile filtration. The components can either be sterilized together or separately if necessary. All components of the media can be present at the start of culture or can optionally be added continuously or batchwise.

The culture temperature is normally between 15° C. and 45° C., preferably at 25° C. to 40° C. and can be kept constant or varied during the experiment. The pH of the medium should be in the range from 5 to 8.5, preferably around 7.0. The culture pH can be controlled during culture by adding basic compounds such as sodium hydroxide, potassium hydroxide, ammonia or ammonia water or acid compounds such as phosphoric acid or sulfuric acid. To control foaming it is possible to use antifoaming agents, such as fatty acid polyglycol esters. For maintaining the stability of plasmids, suitable selectively acting substances, such as antibiotics, can be added to the medium. To maintain aerobic conditions, oxygen or oxygen-containing gas mixtures, such as ambient air, are fed into the culture. The culture temperature is normally at 20° C. to 45° C. Culture is continued until a maximum of the desired product has formed. This target is normally reached within 10 hours to 160 hours.

The fermentation broth is then processed further. Depending on the requirements, the biomass is removed from the fermentation broth completely or partially by separation techniques, such as centrifugation, filtration, decanting or a combination of these methods or can be left in it completely.

If the polypeptides are not secreted into the culture medium, the cells can also be disrupted and the product can be obtained from the lysate by known methods of protein isolation. The cells can optionally be disrupted with high-frequency ultrasound, by high pressure, for example in a French press, by osmolysis, by the action of detergents, lytic enzymes or organic solvents, using homogenizers or by a combination of several of the methods listed.

The polypeptides can be purified by known chromatographic methods, such as molecular sieve chromatography (gel filtration), such as Q-sepharose chromatography, ion exchange chromatography and hydrophobic chromatography, and with other usual methods such as ultrafiltration, crystallization, salting-out, dialysis and native gel electrophoresis. Suitable methods are described for example in Cooper, F. G., Biochemische Arbeitsmethoden [Methods of Biochemical Processing], Verlag Walter de Gruyter, Berlin, New York or in Scopes, R., Protein Purification, Springer Verlag, New York, Heidelberg, Berlin.

For isolating the recombinant protein, it may be advantageous to use vector systems or oligonucleotides that lengthen the cDNA with defined nucleotide sequences and therefore code for modified polypeptides or fusion proteins, which for example serve for easier purification. Suitable modifications of this kind are for example so-called “tags” functioning as anchors, for example the modification known as hexa-histidine anchors, or epitopes that can be recognized as antigens by antibodies (described for example in Harlow, E. and Lane, D., 1988, Antibodies: A Laboratory Manual. Cold Spring Harbor (N.Y.) Press). These anchors can serve for attaching the proteins to a solid carrier, for example a polymer matrix, which can for example be packed in a chromatography column, or can be used on a microtiter plate or on some other support.

At the same time, these anchors can also be used for recognizing the proteins. For recognition of the proteins, in addition usual markers, such as fluorescent dyes, enzyme markers, which form a detectable reaction product after reaction with a substrate, or radioactive markers, can be used, alone or in combination with the anchors for derivatization of the proteins.

### 7. Enzyme Immobilization

In the method described herein, the enzymes according to the invention can be used free or immobilized. An immobilized enzyme is to be understood as an enzyme that is fixed to an inert support. Suitable support materials and the enzymes immobilized thereon are known from EP-A-1149849, EP-A-1 069 183 and DE-OS 100193773 and from the literature references cited therein. Regarding this, full reference is made to the disclosure of these documents. Suitable support materials include for example clays, clay minerals, such as kaolinite, diatomaceous earth, perlite, silicon dioxide, aluminum oxide, sodium carbonate, calcium carbonate, cellulose powder, anion exchanger materials, synthetic polymers, such as polystyrene, acrylic resins, phenol-formaldehyde resins, polyurethanes and polyolefins, such as polyethylene and polypropylene. For preparing the supported enzymes, the support materials are usually used in a finely-divided, particulate form, with porous forms being preferred. The particle size of the support material is usually not more than 5 mm, especially not more than 2 mm (particle-size distribution curve). Similarly, when using dehydrogenase as whole-cell catalyst, a free or immobilized form can be selected. Support materials are for example Ca-alginate, and carrageenan. Enzymes as well as cells can also be crosslinked directly with glutaraldehyde (crosslinking to CLEAs). Corresponding and further methods of immobilization are described for example in J. Lalonde and A. Margolin “Immobilization of Enzymes” and in K. Drauz and H. Waldmann, Enzyme Catalysis in Organic Synthesis 2002, Vol. III, 991-1032, Wiley-VCH, Weinheim.

### EXPERIMENTAL SECTION

Unless stated otherwise, the cloning steps carried out in the context of the present invention, for example restriction cleavage, agarose gel electrophoresis, purification of DNA fragments, transfer of nucleic acids onto nitrocellulose and nylon membranes, linkage of DNA fragments, transformation of microorganisms, culturing of microorganisms, multiplication of phages and sequence analysis of recombinant DNA are carried out as described in Sambrook et al. (1989) op. cit.

### A. General Information

### Materials:

The genomic DNA of Collinsella aerofaciens DSM 3979 (ATCC 25986, former designation Eubacterium aerofaciens) was obtained from the German Collection of Microorganisms and Cell Cultures (DSMZ). UDCA and 7-keto-LCA are starting compounds that are known per se and are described in the literature. All other chemicals were obtained from Sigma-Aldrich and Fluka (Germany). All restriction endonucleases, T4 DNA ligase, Taq DNA polymerase, Phusion DNA polymerase and isopropyl-β-D-1-thiogalactopyranoside (IPTG) were obtained from Fermentas (Germany).

### Media:

LB medium, containing tryptone 10 g, yeast extract 5 g, NaCl 10 g per liter of medium
TB medium, containing tryptone 12 g, yeast extract 24 g, 4 mL glycerol, 10% TB buffer (11.55 g KH₂PO₄, 62.7 g K₂HPO₄, H₂O to 500 mL) per liter of medium
Minimal medium, modified according to Wilms et al., BIOTECHNOLOGY AND BIOENGINEERING, 2001 VOL. 73, No. 2, 95-103

### Expression Vectors and Vector Constructs

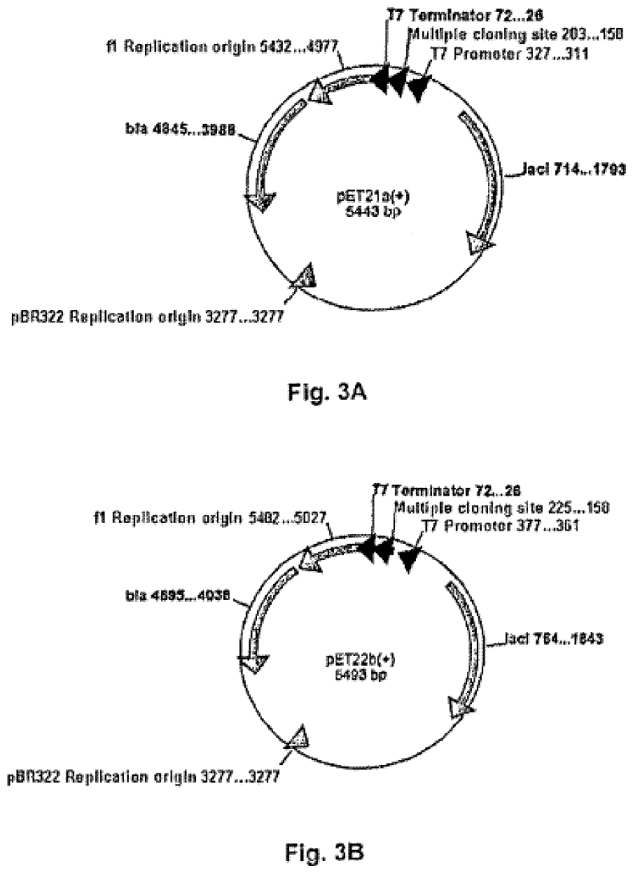

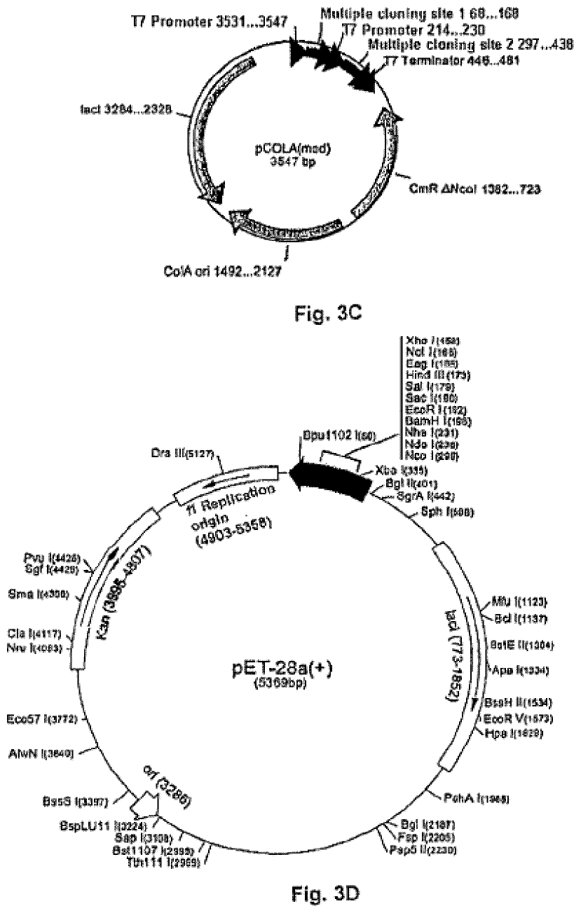

For the expression of recombinant proteins, the following expression vectors that are known per were used:
pET21a(+), (cf. FIG. 3a)
pET22b(+) (cf. FIG. 3b)
pET28a(+) (cf. FIG. 3d) and
pCOLADuet-1
   (in each case Novagen, Madison, Wisconsin, USA).

The vectors pET21a(+) and pET22b(+) each possess a multiple cloning site (MCS), in each case under the control of a T7-promoter with downstream lac-operator and the subsequent ribosomal binding site (rbs). In the C-terminal region of the expression domain there is in each case a T7-terminator. Both plasmids have a ColE1-replicon (pBR322-replicon), an ampicillin resistance gene (bla), an f1-origin and a gene coding for the lac-inhibitor (lacI).

In addition, the pET21a(+)-plasmid has a T7. Tag in the N-terminal region of the MCS and an optional His. Tag C-terminally of the MCS. The pET22b(+)-plasmid has, in the N-terminal region of the MCS, a pelB signal sequence and a His. Tag C-terminally of the MCS.

The pCOLADuet-1 vector has two MCSs, each of which is under the control of a T7-promoter with downstream lac-operator and subsequent ribosomal binding site (rbs). C-terminally of the two MCSs there is a T7-terminator. Moreover, this vector has a gene that codes for the lac-inhibitor and a COLA-replicon (ColA-replicon).

A modified variant of the commercially available pCOLADuet-1 vector was used in the present work. In this modified plasmid variant (designated pCOLA(mod); cf. FIG. 3c)) the kanamycin resistance gene of the original vector was replaced with a chloramphenicol resistance gene. In addition, an NcoI restriction site was removed from the chloramphenicol resistance gene by site-directed point mutagenesis. In the N-terminal region of the first MCS there is a His. Tag, whereas C-terminally of the second MCS there is an S. Tag.

The ColE1-replicons of the pET-plasmids and the COLA-replicon of the pCOLA-plasmid are mutually compatible. This permits simultaneous stable insertion of a pET-plasmid and of a pCOLA-plasmid in Escherichia coli. In this way, combinations of various genes can be cloned into Escherichia coli, without their being located on the same operon. Owing to the different copy numbers of pET-vectors (˜40) and pCOLA-vectors (20-40), it is moreover possible to influence the expression level of cotransformed genes.

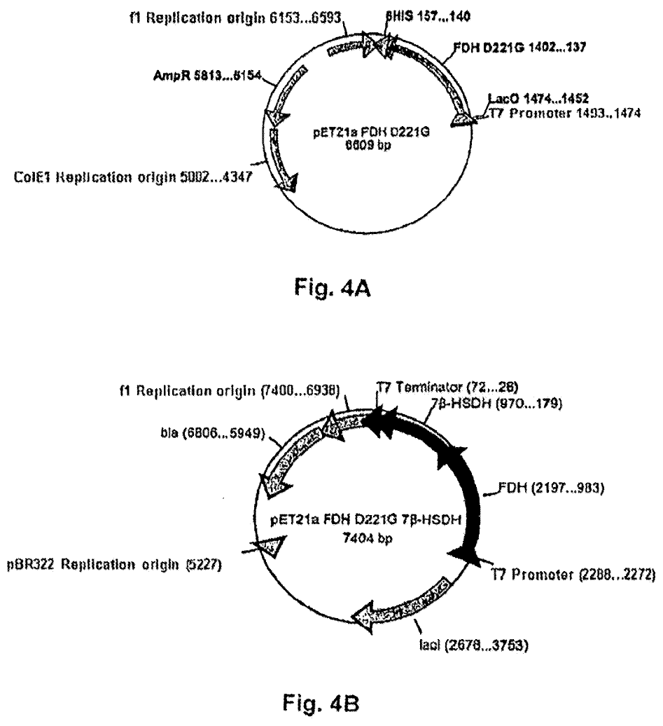

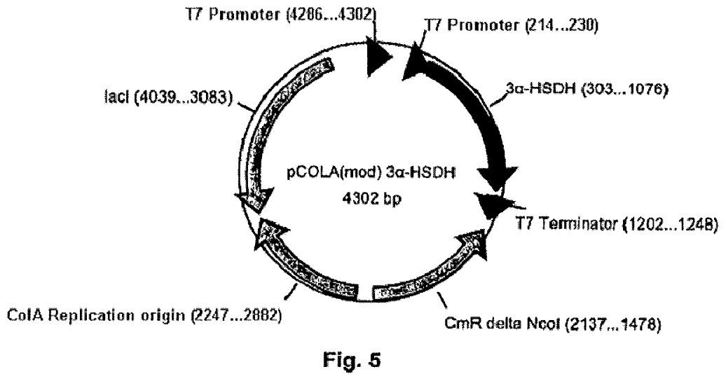

The following vector constructs were used
pET22b(+) 7-HSDH: a pET22b(+) vector into which the 7β-HSDH from Collinsella aerofaciens ATCC 25986 had been cloned via the Nde I and Hind III cleavage sites in the usual way.
pET22b(+) 3α-HSDH: a pET22b(+) vector into which the 3α-HSDH from Comamonas testosteroni had been cloned via the Nde I and EcoR I cleavage sites in the usual way (Oppermann et al., J Biochem, 1996, 241(3): 744-749).
pET21a(+) FDH D221G (cf. FIG. 4a): a pET21a(+) vector into which the formate dehydrogenase from Mycobacterium vaccae N10 had been cloned via the Nde I and EcoR I cleavage sites. With site-directed mutagenesis, the aspartate residue (D) at position 221 (without taking into account methionine in position 1) or position 222 (counting from methionine in position 1; cf. SEQ ID NO: 15, 19, 35) of formate dehydrogenase was replaced with a glycine residue (cf. production example 4, below). Formate dehydrogenase carries, at position 1202 of the nucleotide sequence, a single base deletion, which leads to exchange of the last amino acid valine for an alanine. Simultaneously, this base deletion leads to switching-off of the stop codon and to activation of the His. Tag that was originally outside of the reading frame (cf. SEQ ID NO: 34 and 35).
pET21a(+) 76-HSDH: a pET21a(+) vector, into which the 7β-HSDH from Collinsella aerofaciens ATCC 25986 had been cloned via the Nde I and Xho I cleavage sites in the usual way
pET21a(+) FDH D221G 76-HSDH (cf. FIG. 4b)
pET21a(+) FDH 7β-HSDH(G39A) 3α-HSDH (cf. FIG. 8)
pCOLA(mod) 3α-HSDH (cf. FIG. 5)

### Microorganisms

| Strain | Genotype |
|---|---|
| Escherichia coli DH5α | F⁻ endA1 glnV44 thi-1 recA1 relA1 gyrA96 |
| | deoR nupG Φ80dlacZΔM15 Δ(lacZYA- |
| | argF)U169, hsdR17(r_{K}⁻ m_{K}+), λ− |
| Escherichia coli | F⁻ ompT gal dcm lon hsdS_{B}(r_{B}⁻m_{B}⁻) λ(DE3 |
| BL21(DE3) | [lacl lacUV5-T7 gene 1 ind1 sam7 nin5]) |
| Escherichia coli BL21 | F⁻ ompT gal dcm lon hsdS_{B}(r_{B}⁻m_{B}⁻) λ(DE3 |
| (DE3) hdhA⁻ KanR⁺ | [lacl lacUV5-T7 gene 1 ind1 sam7 nin5]) |
| (7α-HSDH knock-out | hdhA⁻ KanR⁺ |
| strain) (cf. production | |
| example 5) | |

The Escherichia coli strain DH5a (Novagen, Madison, Wisconsin, USA) was multiplied at 37° C. in LB medium containing suitable antibiotics.

The Escherichia coli strain BL21(DE3) (Novagen, Madison, Wisconsin, USA) was multiplied at 37° C. in LB medium containing suitable antibiotics and after induction at OD₆₀₀=0.8 with 0.5 mM IPTG was held at 25° C. and 140 rpm.

### Methods

### 1. Standard Conditions for Determination of 7β-HSDH Activity

The reaction mixture contains a total volume of 1 ml:

| | |
|---|---|
| 880 μl | 50 mM potassium phosphate buffer, pH 8.0 |
| 10 μl | 10 mM UDCA (dissolved in water, pH 8) |
| 10 μl | enzyme solution (in buffer as above, |
| | in the range from 1 to 10 U/ml) |
| 100 μl | 1 mM NADP+ ( in buffer as above) |

The increase in extinction at 340 nm is measured and the activity is calculated as enzyme unit (U, i.e. μmol/min) using the molar extinction coefficient of 6.22 mM⁻¹×cm⁻¹.

### Standard Conditions for Determination of 7β-HSDH Activity According to Production Example 7

The reaction mixture contains a total volume of 1 ml

| | |
|---|---|
| 870 μl | 50 mM potassium phosphate (KPi) buffer, pH 8.0 |
| 100 μl | 100 mM DHCA (dissolved in 50 mM KPi, pH 8) |
| 10 μl | enzyme solution (in buffer as above, |
| | in the range from 2 to 6 U/ml) |
| 20 μl | 12.5 mM NADPH (dissolved in ddH₂O) |

The increase in extinction at 340 nm is measured and the activity is calculated as enzyme unit (U, i.e. μmol/min) using the molar extinction coefficient of 6.22 mM⁻¹×cm⁻¹.

### 2. Determination of Protein by BCA Assay

The samples were mixed with BCA reagent (from Interchim) and incubated at 37° C. for 45 min. The protein content was determined at 562 nm against a calibration curve (BSA) in the concentration range of the assay used.

### 3. Thin-Layer Chromatography

5 to 10 μg of sample was applied to a TLC Film Silica Gel 60 (Merck). Authentic substances were applied as reference. One end of the TLC film was dipped in solvent until the top of the mobile phase was reached. The TLC film was dried and was developed with phosphomolybdic acid.

### 4. Molecular Biology Procedures:

Molecular biology operations are carried out, unless stated otherwise, on the basis of established methods, e.g. described in:
Sambrook, J., Fritsch, E. F. and Maniatis, T. Molecular Cloning: A Laboratory Manual. 2nd ed., Cold Spring Harbor Laboratory, Cold Spring Harbor Laboratory Press, Cold Spring Harbor, N Y, 1989; Ausubel et al. (eds.), Current Protocols in Molecular Biology, John Wiley & Sons, N Y (1993); Kriegler, Gene Transfer and Expression, A Laboratory Manual, Stockton Press, NY (1990).

### B. Examples

### Production Example 1: Identification of 7β-HSDH Activity

The genomic DNA sequence of Collinsella aerofaciens ATCC 25986 was published in the year 2007 by “Washington University Genome Sequencing Center” for the “human gut microbiome project” at GenBank. HSDHs belong to the “short-chain dehydrogenases”. As the biochemical function of the “short-chain dehydrogenases” from Collinsella aerofaciens ATCC 25986 had not been annotated in GenBank, 9 candidates were cloned into vector pET22b+, and then expressed in E. coli BL21(DE3).

For this, 7β-HSDH coding sequences were PCR-amplified. The PCR products were obtained using the genomic DNA of Collinsella aerofaciens ATCC 25986 (DSM 3979) as template and the primers 5′-gggaattcCATATGAACCTGAGGGAGAAGTA-3′ (SEQ ID NO:3) and 5′-cccAAGCTTCTAGTCGCGGTAGAACGA-3′ (SEQ ID NO:4). The NdeI and HindIII cleavage sites in the primer sequences are underlined. The PCR product was purified using the PCR-Purification-Kit (Qiagen) and then cut with the enzymes NdeI and HindIII. The corresponding vector was also cut with NdeI and HindIII. The products were applied to agarose gel, separated, cut out and purified. The cut PCR product and the cut vector were ligated by means of T4-ligase. The ligand was transformed into E. coli DH5a. The resultant vector (contains the gene of 7β-HSDH) was confirmed by sequencing and transformed into E. coli BL21(DE3) and induced with IPTG and expressed.

Expression was carried out in 50 ml LB medium. For preparation of a preculture, a colony on LB-agar plate in 5 ml LB medium (contains corresponding antibiotics) was picked and incubated overnight at 37° C. and 160 rpm. The 50 ml LB medium (contains corresponding antibiotics) was inoculated with 500 μl of preculture. The culture was incubated at 37° C. and 160 rpm. Up to OD600 approx. 0.8, expression was induced by adding 0.5 mM IPTG. After 6 h or overnight, the cells were centrifuged off. The pellets were resuspended in 6 ml potassium phosphate buffer (50 mM, pH 8, contains 0.1 mM PMSF) and disrupted with ultrasound. The cell debris was removed by centrifugation.

For identification of 7β-HSDH activity, the activity was investigated by a photometric method. In a 1 ml cuvette, enzyme and 0.1 mM of test substance (UDCA) were mixed in potassium phosphate buffer (50 mM, pH8). After adding NADPH(NADH) or NADP⁺(NAD⁺), the degradation or formation of NAD(P)H was measured. One enzyme out of the 9 candidates shows activity (60 U/ml) against UDCA in the presence of NADP⁺, but no activity against CA. The NADPH-dependent 7β-HSDH activity of this enzyme was identified.

In the photometric investigation, 7β-HSDH showed activity of 60 U/ml against UDCA, activity of 35 U/ml against 7-keto-LCA and activity of 119 U/ml against DHCA in the presence of NADP⁺ or NADPH. The activity against CA is not detectable.

The gene that codes for 7β-HSDH was subcloned into pET28a+ with His-Tag, to permit rapid purification. This 7β-HSDH with His-Tag was actively expressed in E. coli BL21(DE3) as described above. Purification was carried out with a Talon column. The column was first equilibrated with potassium phosphate buffer (50 mM, pH 8, with 300 mM NaCl). After loading of the cell lysate, the column was washed with potassium phosphate buffer (50 mM, pH 8, with 300 mM NaCl). The 7β-HSDH was eluted with potassium phosphate buffer (50 mM, pH 8, with 300 mM NaCl and 200 mM imidazole). The imidazole in the eluate was removed by dialysis. The yield in purification was 76% with purity of approx. 90%.

### Production Example 2: Preparative-Scale Cloning, Expression and Purification of 7β-HSDH from Collinsella aerofaciens ATCC 25986 and Further Characterization of the Enzyme

### 2.1 Cloning and Production of an Expression Construct

The gene coding for 7β-HSDH was once again amplified from the genomic DNA by PCR and using primers, as described above for production example 1:

The PCR product was once again purified as described above and digested with the restriction endonucleases NdeI and HindIII. The digested PCR product was purified again and cloned into the pET-28a(+) vector using the T4-ligase, to produce an expression vector. The resultant expression construct was then transformed into E. coli DH5a cells. The protein to be expected should have 20 amino acid residues comprising a signal peptide and an N-terminal 6×His-Tag and a thrombin cleavage site. The sequence of the inserted DNA was verified by sequencing.

### 2.2 Overexpression and Purification of 7β-HSDH

E. coli BL21(DE3) was transformed with the expression construct. For this, the E. coli BL21(DE3) strain containing the expression construct was multiplied in LB medium (2×400 ml in 2-liter shaking bottles) containing 30 μg/ml kanamycin. The cells were harvested by centrifugation (10.000×g, 15 min, 4° C.). The pellet was resuspended in 20 ml phosphate buffer (50 mM, pH 8, containing 0.1 mM PMSF). The cells were disrupted by ultrasound treatment for 1 minute with constant cooling (40 W power, 40% working interval and 1 min pause) using Sonifier 250 ultrasonic equipment (Branson, Germany). Lysis was repeated three times. The cell extract was centrifuged (22.000×g, 20 min, 4° C.). The supernatant was loaded on a Talon column (Clontech, USA), equilibrated with loading buffer (50 mM potassium phosphate, 300 mM NaCl, pH 8). The procedure was carried out at 24° C. Unbound material was washed away by washing the column with loading buffer (3 column volumes). Weakly binding protein was removed by washing with washing buffer (20 mM imidazole in the loading buffer; 3 column volumes). The His-Tag-7β-HSDH protein was eluted with elution buffer (200 mM imidazole in the loading buffer). The eluate was dialyzed overnight in a dialysis tube with a molecular exclusion limit of 5 kDa (Sigma, USA) in 2 liters of potassium phosphate buffer (50 mM, pH 8) at 4° C. Finally the sample was transferred to a new tube and stored at −20° C. for further analysis. The protein concentration was determined using a BCA Test Kit (Thermo, USA) according to the manufacturer's instructions. In addition the sample was analyzed by 12.5% SDS-PAGE and staining with Coomassie Brilliant Blue. The purity of the protein was determined densitometrically using Scion Image Beta 4.0.2 (Scion, USA).

### 2.3 Gel Filtration

Gel filtration was carried out on a Pharmacia ÄKTA protein purification system, in order to determine the molecular weight of 7β-HSDH. The purified enzyme was applied on a Sephadex G-200 column, which had been equilibrated beforehand with 50 mM Tris-HCl (pH 8), containing 200 mM sodium chloride. The protein was eluted with the same buffer at a flow rate of 1 ml/min. The molecular weight of 7β-HSDH was determined by comparing its elution volume with that of protein standards (serum albumin (66 kDa), α-amylase from Aspergillus oryzae (52 kDa), pig pancreas trypsin (24 kDa) and hen's egg lysozyme (14.4 kDa)).

### 2.4 Enzyme Assay and Kinetic Analysis

The reaction mixture for the enzyme assay contained, in a total volume of 1 ml, 50 μmol potassium phosphate (pH 8), 0.1 μmol NAD(P)H or NAD(P)⁺, substrates and protein. The reaction mixture was contained in cuvettes with a light path length of 1 cm. The 7β-HSDH activity was determined by recording the variation in NAD(P)H concentration against the extinction at 340 nm using a spectrophotometer (Ultraspec 3000, Pharmacia Biotech, Great Britain). The enzyme activities were determined as enzyme units (U, i.e. μmol/min) using the molar extinction coefficient of 6.22 mM⁻¹×cm⁻¹ at 25° C. Several different measurements were performed with the variables substrate, coenzyme, concentration, pH, buffer and incubation temperature. The kinetic constants were determined using standard methods.

### 2.5 Biotransformation of 7-Keto-Lithocholic Acid by 7β-HSDH

The transformation of 7-keto-LCA by 7β-HSDH was carried out in order to verify the biochemical function of 7β-HSDH. 0.4 g 7-keto-LCA was suspended in 10 ml potassium phosphate buffer (50 mM, pH 8) and the pH was adjusted to pH 8 by adding 2 M sodium hydroxide. 0.2 ml isopropanol, 100 U 7β-HSDH and 80 U alcohol dehydrogenase (ADH-TE) from Thermoanaerobacter ethanolicus (kindly donated by Dr. K. Momoi, ITB University, Stuttgart) and 1 μmol NADP⁺ were added. The same buffer was added, to give a total reaction volume of 20 ml. The reaction mixture was incubated at 24° C. and stirred for 24 hours. During this, NADPH was regenerated with ADH via oxidation of 2-propanol. The product was acidified with 1 ml of 2 M hydrochloric acid and was extracted 5× with 5 ml ethyl acetate. The organic solution was then distilled.

### 2.6 Determination of Chromatographic Product

An HPLC analysis was carried out on a column of the Purospher® STAR RP-18 type (Hitbar® RT 125-4 Pre-Packed Column, Purospher® STAR RP-18 endcapped, Merck, Germany), provided with a precolumn of the LiChroCART® STAR RP18 type (endcapped, Merck, Germany) on an HPLC system LC20AD (Shimadzu, Japan) at a flow rate of 1 ml/min. The mobile phase consisted of two eluents. Eluent A contained acetonitrile and eluent B contained distilled water (pH 2.6, adjusted with orthophosphoric acid, 85%). The following gradient was used: A 35% (8 min)-35%-43% (1% min⁻¹)-43%-70% (1% min⁻¹)-70% (5 min)-70%-35% (17.5% min⁻¹)-35% (5 min); eluent A 65% (8 min)-65%-57% (1% min⁻¹)-57%-30% (1% min⁻¹)-30% (5 min) 30%-65% (17.5% min⁻¹)-65% (5 min). 20 μl samples (1 mg/ml) were analyzed. Authentic UDCA, 7-keto-LCA and CDCA were used at the same concentration as standards. Recording was carried out by UV detection at 200 nm.

### 2.7 Sequence Alignment and Phylogenetic Analysis

Multiple sequence alignments were produced using the Clustal X-Software (Thompson et al., 1997, Nucleic Acid Research 25: 4876-82) and modified using the Jalview software (Clamp et al., 2004, Bioinformatics 20: 426-7). The phylogenetic tree was produced using the program TreeView 1.6.6 (Roderic 2001).

### 2.8 Test Results:

### 2.8.1 Identification of 7β-HSDH Activity in a Preparative Biotransformation

To confirm the function of the enzyme, a biotransformation of 7-keto-LCA was carried out at the 10-ml scale, wherein the isolated enzyme was used in combination with ADH for regeneration of NADPH using 2-propanol, as already described. The HPLC analysis showed that UDCA was the only reaction product produced by the enzyme (90% conversion). CDCA (retention time 19.4 min) was not detected in the reaction mixture. The result shows that the enzyme is an NADPH-dependent 7β-HSDH and is capable of selective reduction of the 7-carbonyl group of 7-keto-LCA to a 7β-hydroxyl group.
Retention time UDCA: 15.5 min
Retention time 7-keto-LCA: 18.3 min

### 2.8.2. Purification and Gel Filtration

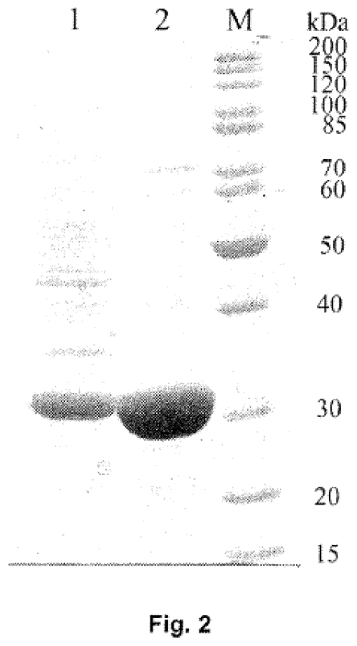

After cloning the 7β-HSDH gene from Collinsella aerofaciens DSM 3979 into the expression vector pET28a(+) and subsequent overexpression, a fusion protein provided with a His-Tag on the N-terminus was obtained with a 7β-HSDH yield of 332.5 mg (5828 U) per liter of culture. The 7β-HSDH provided with the His-Tag was purified in one step by immobilized metal ion affinity chromatography (purity >90%, yield 76%, cf. FIG. 2.). The main bands of lanes 1 and 2 represent the expected expression product at 30 kDa, which corresponds to the predicted molecular weight derived from the amino acid sequence of the gene. However, a molecular weight of 56.1 kDa is found for 7β-HSDH by gel filtration. This proves the dimeric nature of the 7β-HSDH from Collinsella aerofaciens DSM 3979.

### 2.8.3. Sequence Alignments

The amino acid sequence of the 7β-HSDH used according to the invention was compared with known HSDH sequences (alignment not shown). The observed sequence similarity indicates that the enzyme according to the invention belongs to the family of short-chain dehydrogenases (SDR). It is known that SDRs have very low homology and sequence identity (Jornvall, H., B. Persson, M. Krook, S. Atrian, R. Gonzalez-Duarte, J. Jeffery, and D. Ghosh. 1995. Short-chain dehydrogenases/reductases (SDR). Biochemistry 34: 6003-13 and Persson, B., M. Krook, and H. Jornvall. 1991. Characteristics of short-chain alcohol dehydrogenases and related enzymes. Eur J Biochem 200: 537-43). However, the sequence alignment clearly shows the conserved domains in the SDR primary structure. The N-terminal motif Gly-X-X-X-Gly-X-Gly (corresponding to Gly-41, Gly-45 and Gly-47, numbering corresponding to the alignment) corresponds to the characteristic dinucleotide binding motif of the SDR superfamily. Furthermore, three strongly conserved residues Ser-177, Tri-190 and Lys-194 are observed, which correspond to the catalytic triad of the SDR enzymes.

### 2.8.4. Phylogenetic Analysis

7α-HSDHs from Clostridium sordellii, Brucella melitensis and Escherichia coli belong to the same subgroup. The two 3α-HSDHs show a more pronounced similarity than other HSDHs. Interestingly, the prokaryotic 7β-HSDH is related to the animal 11β-HSDH subgroup, comprising Cavia porcellus, Homo sapiens and Mus musculus.

### 2.8.5. Kinetic Constants

Kinetic equilibrium analyses were carried out, in order to determine the absolute values for Vₘₐₓ and K_{M} for UDCA, 7-keto-LCA, DHCA, NADP⁺ and NADPH by Lineweaver-Burk plots. The following table presents all kinetic data for the substrates and coenzymes tested, which were obtained from substrate saturation curves and reciprocal plots. The Vₘₐₓ, K_{M} and K_{cat} values for all substrates and coenzymes are in the same region, whereas the K_{M} value for DHCA was significantly higher than with the other substrates, possibly caused by the low solubility in water. The enzyme is NADPH-dependent and kinetic constants could not be determined for NAD⁺ and NADH owing to the very low activity.

Summary of Kinetic Constants for 7β-HSDH from Collinsella aerofaciens DSM 3979.

| | K_{M} | Vₘₐₓ | k_{cat} |
|---|---|---|---|
| | (μM) | (U/mg protein)^{b)} | (1 μmol/(μmol × min)) |
| NADP⁺ | 5.32 | 30.58 | 944.95 |
| NADPH | 4.50 | 33.44 | 1033.44 |
| UDCA | 6.23 | 38.17 | 1179.39 |
| 7-Keto-LCA | 5.20 | 30.77 | 950.77 |
| DHCA | 9.23 | 28.33 | 875.35 |
| NAD⁺ | —^{a)} | — | Traces |
| NADH | — | — | Traces |

^{ta)}could not be determined owing to the very low activity
^{b)}1 U = 1 μmol/min

### 2.8.6. Optimal pH

In addition, the 7β-HSDH activity was determined with purified enzyme for various substrates as a function of the pH. For the oxidation of UDCA with 7β-HSDH, an optimal activity was observed in the range from pH 9 to 10 with gradual decline on the acidic side. In contrast, for the reduction of DHCA and 7-keto-LCA by 7β-HSDH, there was an optimal activity in the range from pH 4 to 6 with a sharp decline on the acidic side and a gradual decline on the alkaline side. Different buffers only have a slight influence on the activity of 7β-HSDH at identical pH.

### 2.8.7. Thermal Stability

The NADP-dependent 7β-HSDH used according to the invention shows the following stability behavior: after 400 min the activity at 30° C. was about 30% lower than at 23° C. At 30° C., the enzyme was completely inactivated after 1500 min, whereas at 23° C. and 1500 min the residual activity was 20%. No significant activity loss was observed during storage at −20° C. in potassium phosphate buffer (50 mM, pH 8) over a period of some months after repeated freezing and thawing.

### Production Example 3: Production of 7β-HSDH Mutants and Characterization Thereof

Position 39 of the amino acid sequence (comprising start methionine) (cf. SEQ ID NO:2) was mutated.

### 3.1 Primers

The mutagenesis primers stated below were used for the site-directed mutagenesis of 7β-HSDH. The primers were selected based on the 7β-HSDH gene sequence, so that they bring about the desired amino acid exchange. It was borne in mind that the base to be mutated is localized centrally in the primer, and that the melting points of the primer pairs are in the same region.

The primer pair 7beta_mut_G39A_fwd and 7beta_mut_G39A_rev was used for preparing the G39A mutant. The primer pair 7beta_mut_G39S_fwd and 7beta_mut_G39S_rev was used for preparing the G39S mutant.

Glycine → Alanine
Forward:
SEQ ID NO: 9)
7beta_mut_G39A_fwd: CGTCGTCATGGTCGCCCGTCGCGAGG.
Reverse:
SEQ ID NO: 10)
7beta_mut_G39A_rev: CCTCGCGACGGGCGACCATGACGACG.
Glycine → Serine
Forward:
SEQ ID NO: 11)
7beta_mut_G39S_fwd: CGTCGTCATGGTCAGCCGTCGCGAGG.
Reverse:
SEQ ID NO: 12)
7beta_mut_G39S_rev: CCTCGCGACGGCTGACCATGACGACG.

### 3.2 PCR Program

In the reaction, first a 2-min initial denaturation step was carried out at 98° C. Then 25 cycles of denaturation (30 s at 98° C.), primer hybridization (2.5 min at 58° C.) and elongation (6 min at 72° C.) were carried out. As the last step, a final elongation of 15 min was carried out at 72° C. before the polymerase chain reaction was stopped by cooling to 4° C.

### 3.3 PCR Assay

| | | |
|---|---|---|
| HF buffer (5x) | 4 | μl |
| dNTP-mix (10 mM) | 0.4 | μl |
| Forward primer (10 μM) | 2 | μl |
| Reverse primer (10 μM) | 2 | μl |
| Template | 1 | μl |
| Phusion polymerase (2 U μL⁻¹) | 0.2 | μl |
| DMSO | 1 | μl |
| ddH₂O | 9.4 | μl |
| | 20 | μl |

A pET22b vector with 7β-HSDH was used as template.

### 3.4 Procedure

To allow targeted exchange of amino acids in protein sequences, the DNA sequence of the corresponding gene is submitted to site-directed mutation. For this, mutually complementary primers are used, which bear the desired mutation in their sequence. N6-adenine-methylated, double-stranded plasmid DNA, which bears the gene to be mutated, serves as template. N6-adenine-methylated plasmid DNA is isolated from dam⁺ E. coli strain such as for example E. coli DH5.

The polymerase chain reaction is carried out as described above. The primers are lengthened complementarily to the template, so that plasmids with the desired mutation are formed, which have a strand break. Unlike other PCR reactions, in this case the increase in DNA yield is only linear, as newly formed DNA molecules cannot serve as template for the PCR reaction.

On completion of the PCR reaction, the parental, N6-adenine-methylated DNA is digested by the restriction enzyme DpnI. This enzyme has the particular feature that it restricts nonspecifically N6-adenine-methylated DNA, but not the newly formed, nonmethylated DNA. Restriction was carried out by adding 1 μL DpnI to the PCR reaction mixture and incubating for 1 h at 37° C.

10 μl of this preparation was used for the transformation of 200 μl of chemically-competent DH5a cells. After plasmid isolation, successful mutation was confirmed by sequencing.

### 3.5 Characterization

For each of the mutants and for the wild-type enzyme, kinetic measurements were carried out in the microtiter plate photometer, recording the conversion rates of the enzymes both with constant substrate concentrations with variation of cofactor concentrations and vice versa. To determine the specific enzyme activity, the enzyme concentrations in the cell lysate were determined by densitometry.

To determine the dependence of the substrate conversion rate on the substrate concentration, a cofactor concentration of 100 μM NADPH relative to the reaction volume was used, while the substrate concentration was varied over a range from 7 μM to 10 mM dehydrocholic acid with 31 different concentrations (in each case relative to the reaction mixture).

To determine the dependence of the substrate conversion rate on the cofactor concentration, a substrate concentration of 0.3 mM dehydrocholic acid relative to the reaction volume was used, whereas the cofactor concentration was varied over a range from 25 UM to 100 μM NADPH with 8 different concentrations of the wild-type protein or over a range from 6 μM to 100 μM NADPH with 16 different concentrations for both mutants (in each case relative to the reaction mixture). In these measurement series, the reaction rate was determined by linear regression over the first 4 measurements (0 s-18 s).

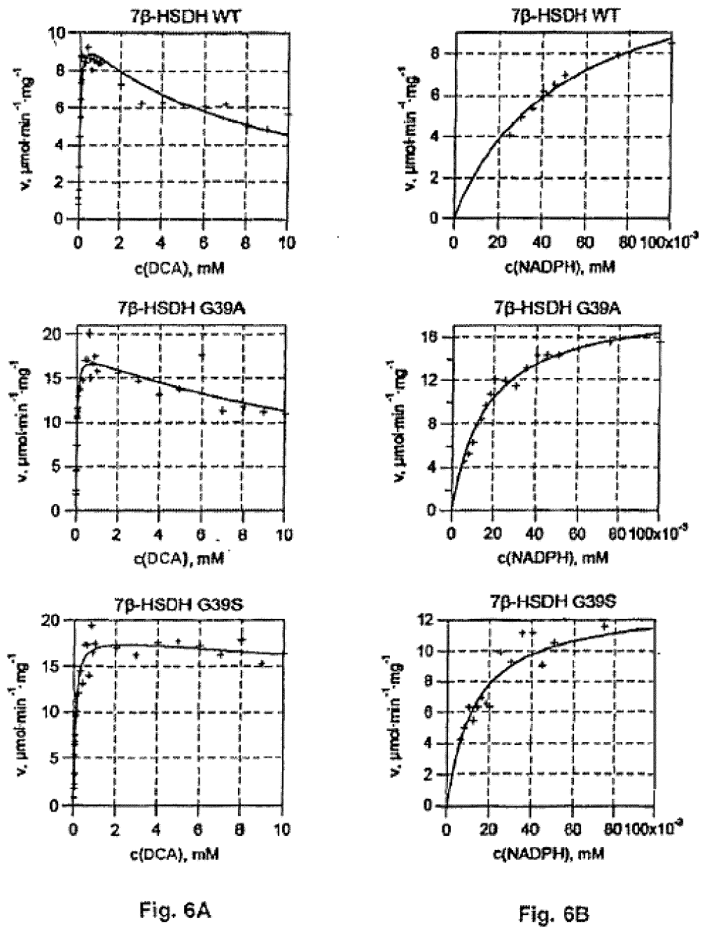

The data obtained were evaluated with IGOR Pro. From the plots of substrate or cofactor concentration against the specific enzyme activity, the typical curve of Michaelis-Menten kinetics can be seen for the measurement series at constant substrate concentration and variation of cofactor concentration. From the plots of the measurement series with constant cofactor concentration and variation of substrate concentration, however, typical curves of Michaelis-Menten kinetics with substrate inhibition can be seen (cf. FIG. 6). For this reason, the classical Michaelis-Menten model was used for evaluating the measurement series at constant substrate concentration and variation of cosubstrate concentration, and the Michaelis-Menten model with substrate inhibition was used for the measurement series with constant cosubstrate concentration and variation of substrate concentration.

vₘₐₓ, Kₘ and Kᵢ were determined by nonlinear regression.

$$v = {v}_{max} \text{?} \frac{{c}_{S}}{{K}_{m} + ( 1 + \frac{{c}_{S}}{{K}_{i}} ) {c}_{S}}$$

$$\text{?} \text{indicates text missing or illegible when filed}$$

v specific enzyme activity, U mg⁻¹=μmol min⁻¹ mg⁻¹
vₘₐₓ maximum specific enzyme activity, U mg⁻¹=μmol min⁻¹ mg⁻¹
cₛ substrate or cofactor concentration, mol L⁻¹
Kₘ semisaturation concentration, mol L⁻¹
Kᵢ inhibition constant, mol L⁻¹
   The parameters found are shown in the table.

TABLE
Parameters found for the three different 78-HSDH variants

| | K_{m, DCS}, | K_{i, DCS}, | K_{m, NADPH}, | Vₘₐₓ, |
|---|---|---|---|---|
| | μM | mM | μM | U/mg |
| 7β-HSDH WT | 31 ± 5 | 8.6 ± 1.5 | 46 ± 7 | 14.6 ± 1.0 |
| 7β-HSDH G39A | 33 ± 6 | 17 ± 4 | 16.4 ± 1.9 | 21.0 ± 1.1 |
| 7β-HSDH G39S | 75 ± 9 | 80 ± 50 | 13.1 ± 2.9 | 20.7 ± 1.0 |

On examining the measured data, it is clear that 7β-HSDH displays substrate inhibition via dehydrocholic acid, which is strongest for the wild-type protein and weakest with the G39S mutant. Especially in the case of this last-mentioned protein, it may be asked whether substrate inhibition occurs at all, as indicated by the high Kᵢ and its large error. The semisaturation concentrations for dehydrocholic acid are in the two-digit micromolar range. Whereas these do not differ significantly for the wild-type protein and the G39A mutant, at 31±5 UM and 33±6 UM respectively, for the G39S mutant this value, at 75±9 UM, is roughly double the value for the other two enzymes. In the case of the semisaturation concentrations for NADPH, lower values are found for the mutant enzymes than for the wild-type enzyme (46±7 UM in the wild-type versus 16.4±1.9 UM for the G39A mutant and 13.1±2.9 UM for the G39S mutant).

For both mutants, the values for vₘₐₓ are roughly 1.5 times the value of the wild-type protein (21.0±1.1 U/mg for the G39A mutant and 20.1±1.0 U/mg for the G38S mutant versus 14.6±1.0 U/mg for the wild-type protein). In contrast to the classical Michaelis-Menten model, however, for the Michaelis-Menten model with substrate inhibition the vₘₐₓ values are not to be regarded as maximum attainable specific enzyme activities, since with increasing substrate concentration the specific enzyme activities do not approach vₘₐₓ asymptotically, but decrease again. The maximum attainable enzyme activities, i.e. the specific enzyme activities at the maxima of the kinetic curves, are ˜13 U/mg for the wild-type protein and ˜19 U/mg (G39A) and ˜20 U/mg(G39S) for the mutant proteins. The enzyme activities that can be reached at the substrate concentrations used for whole-cell biotransformation are more interesting than the maximum attainable enzyme activities. As an example, these are to be compared for the substrate concentration of 10 mM dehydrocholic acid, and are ˜6.7 U/mg for the wild-type protein, ˜13 U/mg for the G39A mutant and ˜18 U/mg for the G39S mutant. At 10 mM substrate concentration, the G39A mutant would accordingly be twice as active as the wild-type protein, and the G39S mutant would even have about 3 times its activity. These differences should be even more pronounced at higher substrate concentrations, as the wild-type protein displays stronger substrate inhibition than the G39A mutant, and the G39A mutant is in its turn more strongly substrate-inhibited than the G39S mutant.

### Production Example 4: Production and Characterization of the FDH D221G Mutant

4.1 Cloning pET21a(+) FDH
4.1.1 PCR Amplification of Mycobacterium vaccae Formate Dehydrogenase

The template used for the amplification is genomic DNA of Mycobacterium vaccae, which was obtained from the German Collection of Microorganisms and Cell Cultures (Deutsche Sammlung von Mikroorganismen und Zellkulturen GmbH, DMSZ), Brunswick. The primers for the amplification were

(SEQ ID NO: 23)
fdh_for (5′-CGATCATATGGCAAAGGTCCTGTGCGTTC-3′)
and
(SEQ ID NO: 24)
fdh_rev (5′-GCTAGAATTCTCAGCCGCCTTCTTGAACT-3′),

obtained from Eurofins MWG GmbH, Ebersberg. The recognition sites for the restriction enzymes are underlined. The rev-primer contains the EcoRI cleavage site and the for-primer contains the NdeI cleavage site.
The PCR assays and PCR programs are shown in the following table.

TABLE
PCR assay for the amplification of formate
dehydrogenase from Mycobacterium vaccae

| Component | Volume [μl] |
|---|---|
| 10× Taq buffer (with Mg²⁺) | 5 |
| dNTPs (10 mM) | 1 |
| Fdh_for (100 μM) | 0.5 |
| Fdh_rev (100 μM) | 0.5 |
| Template DNA (≥100 ng/μL) | 1 |
| Taq DNA polymerase (5 U/mL) | 0.5 |
| Distilled water | 41.5 |

TABLE
PCR program for the amplification of formate dehydrogenase
from Mycobacterium vaccae

| | Cycle | | | |
|---|---|---|---|---|
| Segment | number | Denaturation | Annealing | Elongation |
| 1 | 1 | 94° C., 2 min | | |
| 2 | 5 | 94° C., 30 s | 55.6° C., 30 s | 72° C., 75 s |
| 3 | 25 | 94° C., 30 s | 58.6° C., 30 s | 72° C., 75 s |
| 4 | 1 | | | 72° C., 75 s |

4.1.2 Restriction Digestion of pET21a(+) and FDH PCR Product

5 μl 10× NEBuffer EcoRI, 2.5 μl NdeI (20 U/mL) and 2.5 μl EcoRI (20 U/mL) (in each case New England Biolabs, Frankfurt) were added to 1-5 μg of DNA (pET21a(+) or FDH PCR product) dissolved in water, and made up to 50 μl total volume with distilled water. The preparations were in each case incubated for 1 h at 37° C. Then the cut DNA fragments were applied to 1% agarose gel (1% (w/v) agarose, 0.05% (v/v) ethidium bromide) and the DNA fragments were separated electrophoretically for 55 minutes at 120 V. Then the bands of the correct size (1.2 kb for the FDH-gene, 5.4 kb for the pET21a(+)-plasmid) were cut out of the agarose gel with a scalpel and were isolated using the QIAquick Gel Extraction Kit (QIAGEN, Hilden) according to the manufacturer's protocol

4.1.3 Ligation of Cut pET21a(+) and FDH

1 μl T4 ligase (3 U/μL) and 1 μl 10× ligase buffer (in each case New England Biolabs, Frankfurt) were added to 100 ng of cut vector DNA and 111 ng of cut FDH DNA and made up to a total volume of 10 μl with distilled water. The ligation preparation was incubated overnight at 4° C.

4.1.4 Transformation of the Ligation Preparation into Chemically Competent E. coli DH5a

At the end of the ligation step, the 10 μL ligation preparation is added to 200 μl of chemically competent E. coli DH5a prepared according to the standard protocol. Next there is a 30-min incubation step on ice, followed by heat shock at 42° C. (90 seconds). Then 600 μl of sterile LB medium is added to the transformation preparation and the cells are incubated at 200 rpm and 37° C. in a shaking incubator for 45 minutes. In the next step, the preparation is centrifuged at 3000 rpm for 60 seconds in a benchtop centrifuge, 700 μl of the supernatant is discarded, the cells are resuspended in the remaining supernatant and plated out on an LB-agar plate with 100 mg/l ampicillin. The agar plate is then incubated overnight at 37° C.

4.2. Production of pET21a(+) FDH D221G

The production of an FDH mutant, which can regenerate not only NADH, but also NADHP, will be explained in more detail with the mutant D221G.

Aspartate (D) 221 is an amino acid with a negatively-charged large side chain, which is located directly next to the arginine (R) residue, by which NADP⁺ is to be bound. This can lead to repulsion of the phosphate group in the NADP⁺, which is also negatively charged. The aspartate is therefore replaced with the small uncharged amino acid residue glycine (G).

### 4.2.1 Primers Used

mt1:
(SEQ ID NO: 30)
5′- C CTG CAC TAC ACC GGC CGT CAC CGC CTG C-3′
NI_fdh_R:
(SEQ ID NO: 31)
5′ -GCTCGAATTCTCAGACCGCCTTC--3′

### 4.2.2 Procedure

First a set of two complementary megaprimers was produced using the mt-primer and the primer NI_fdh_R.

Plasmid pET21a(+)FDH was used as template. The PCR program used is shown in the following table:

TABLE
Megaprimer PCR program

| Segment | Cycle number | Denaturation | Annealing | Elongation |
|---|---|---|---|---|
| 1 | 1 | 94° C., 2 min | | |
| 2 | 30 | 94° C., 30 s | 60° C., 30 s | 72° C., 40 s |
| 3 | 1 | 72° C., 5 min | | |

By combining primer mt1 with the primer NI_fdh_R, the length of the megaprimer becomes 650 bp. With this PCR product of the first PCR, gel electrophoresis and isolation of the desired band from the gel are carried out. A second PCR is carried out as whole plasmid PCR with the megaprimers as primers and the plasmid DNA (pETfdh) as template. The reaction mixture and the temperature scheme for the whole plasmid PCR are shown in the following tables. The 2× EZClone enzyme mix, the EZClone solution 1, the 1.1 kb marker and the DpnI were obtained from the GeneMorph II EZClone Domain Mutagenesis Kit (Stratagene).

TABLE
Preparation for a MEGA WHOP PCR
(total volume 50 μL)

| Component | |
|---|---|
| Megaprimer | 250 ng (~2.5 μL |
| | from a standard-PCR) |
| Template (pETfdh) | 50 ng |
| 2× EZClone enzyme mix | 25 μL |
| EZClone solution 1 | 3 μL |
| distilled water | to 50 μL |

The first step in the PCR program (68° C., 5 min) is for removing the bases appended nonspecifically by the Taq-polymerase by the 3′->5′ exonuclease activity of the polymerase used in the MEGA WHOP PCR.

TABLE
MEGA WHOP PCR program

| Segment | Cycle number | Denaturation | Annealing | Elongation |
|---|---|---|---|---|
| 1 | 1 | | | 68° C., 5 min |
| 2 | 1 | 95° C., 1 min | | |
| 3 | 25 | 95° C., 50 s | 60° C., 50 s | 68° C., 13 min |

The PCR product is a double-stranded plasmid with single-strand breaks, which are only closed in E. coli. 10 U DpnI were added to the 50 μL PCR product and the preparation was incubated at 37° C. for two hours. DpnI only degrades methylated DNA, i.e. the template DNA used, but not the megaprimer or the synthesized plasmid. The template plasmid must be produced with a dam⁺ strain (such as DH10B or JM109), to obtain methylated starting DNA.

### 4.3 Expression and Isolation of the Mutant D221G

First an LB preculture was inoculated from frozen stored material or with a colony from an agar plate and the preculture was incubated overnight. Incubation was carried out at 37° C. and 250 rpm. The OD of the preculture was determined and the culture was inoculated to an OD of 0.1. On reaching an OD of 0.5-1 (after about 2.5 h), it was induced with IPTG (final concentration 1 mM). The cells were harvested three hours after induction.

The cells were disrupted mechanically with glass beads. For this, 0.5 mL of glass beads were added to 0.5 mL of cell sample in a 1.5-mL reaction vessel and the reaction vessel was shaken for 3 min at maximum frequency (30 s⁻¹) in a Retsch vibratory mill. After cell maceration, the glass beads were centrifuged off (2 min, 13000 rpm). Before and after maceration, the culture was put on ice, to minimize protein denaturation through heating of the sample in the vibratory mill. This maceration protocol is optimized for the use of samples frozen at −20° C.

### 4.4 Characterization of the Mutant D221G

### Summary of Kinetic Constants of FDH at pH 6, 7 and 8.

| | | | | Specific | K_{M} | K_{M} |
|---|---|---|---|---|---|---|
| | | | | activity | (mM) | (mM) |
| Enzyme | pH | Substrate | Cofactor | (U × mg⁻¹) | substrate | cofactor |
| FDH | 6 | Sodium formate | NAD⁺ | 4.6 | 46.86 | 0.83 |
| | 6 | Sodium formate | NADP⁺ | 1 | | 0.3 |
| | 7 | Sodium formate | NAD⁺ | 5.1 | 45.56 | 0.61 |
| | 7 | Sodium formate | NADP⁺ | 1.2 | | 0.51 |
| | 8 | Sodium formate | NAD⁺ | 4.7 | 59.26 | 0.52 |
| | 8 | Sodium formate | NADP⁺ | 1 | | 1.01 |

The data prove that the mutant produced can utilize both NAD⁺ and NADP⁺ as cofactor.

### Production Example 5: Production of an E. coli 7α-HSDH Knockout Mutant (E. coli BL21 (DE3) hdhA⁻KanR⁺)

The target is deletion of the disturbing 7α-HSDH activity in the expression strain E. coli BL21 (DE3).

With the method described below, an antibiotic resistance gene is inserted in the target gene of 7α-HSDH, so that the target gene is switched off.

5.1 Sequence Information for 7α-HSDH from E. coli BL21(DE3)

Amino acid sequence: (SEQ ID NO:26) Nucleotide sequence (SEQ ID NO:25) Accession: NC_012971 REGION: 1642470 . . . 1643237

### 5.2 Primers Used

The following primers were prepared for switching off the 7α-HSDH from E. coli BL21(DE3):

### Primer for the Retargeting of the LI.LtrB Intron:

467|468a-IBS
(SEQ ID NO: 27)
AAAAAAGCTTATAATTATCCTTATAGGACGTCATGGTGCGCCCAGATAGG
GTG
467|468a-EBS1d
(SEQ ID NO: 28)
CAGATTGTACAAATGTGGTGATAACAGATAAGTCGTCATGTTTAACTTAC
CTTTCTTTGT
467|468a-EBS2
(SEQ ID NO: 29)
TGAACGCAAGTTTCTAATTTCGGTTTCCTATCGATAGAGGAAAGTGTCT
Insertion
Location
467|468a
GCAGCTTTAGATGATGCATAGGAAGTCATG - intron -
TTTATATTTTTATTT

### 5.3 Preparation of the Knockout Mutant

The knockout mutant was prepared using the kit TargeTron™ Gene Knockout System from Sigma Aldrich according to the manufacturer's instructions. The QIAquick PCR Purification Kit from Qiagen was used for purifying the PCR product according to step B.6. of the TargeTron™ Gene Knockout System.

Ligation of the HindIII/BsrGI-digested intron PCR product into the linearized pACD4K-C vector was carried out as follows: the reaction was carried out overnight at 16° C.

### 20 μl Preparation:

| | |
|---|---|
| 2 μl | pACD4K-C linear vector (40 ng) |
| 6 μl | HindIII/BsrGI-digested intron PCR product |
| 2 μl | ATP (10 mM) |
| 2 μl | ligase buffer (10×) (Fermentas) |
| 2 μl | T4-ligase (Fermentas) |
| 6 μl | H₂O |

5 μl of ligation reaction solution was added to 200 μl of chemically-competent cells of E. coli BL21 (DE3) and incubated on ice for 20 min. Further transformation was carried out as described by the manufacturer.

The transformation preparations were plated out on LB-agar plates, containing 33 μg/mL kanamycin. Kanamycin-resistant cells were picked and these were in each case inoculated over several nights in 5 ml LB overnight cultures (in each case with 5 μl of a kanamycin solution (33 mg/ml)). Finally a 200 ml LB culture (with 200 μl kanamycin solution (33 mg/mL)) was inoculated with an overnight culture and was incubated for 5 h at 37° C. and 180 rpm in a shaking incubator. Then the temperature was raised to 42° C. for 1 hour. This culture was used for inoculating a 5 mL LB overnight culture (in each case with 5 μl of a kanamycin solution (33 mg/mL)). After incubation overnight at 37° C. and 180 rpm, the culture was streaked on an LB-agar plate with 33 μg/mL kanamycin. After overnight incubation at 37° C., colonies were picked and streaked on LB-agar plate with 33 μg/mL kanamycin and 34 μg/mL chloramphenicol.

After overnight incubation at 37° C., chloramphenicol-sensitive mutants were found. This is necessary in order to confirm the loss of the plasmid that is carried by the inducible knockout system and is no longer required after successful knockout.

### 5.4. Detection of the Knockout

The 7α-HSDH gene was amplified by colony PCR with the primers

7alpha-ko-check_fwd
SEQ ID NO: 32
(5′-TTAATTGAGCTCCTGTACCCCACCACC-3′)
and
7alpha-ko-check_rev
SEQ ID NO: 33
(5′-GTGTTTAATTCTGACAACCTGAGACTCGAC-3′).

The resultant fragment had a length of approx. 2.5-3 kb and was sequenced with the primer 7alpha-ko-check_fwd. The sequencing showed that the DNA sequence of 7α-HSDH is interrupted by an insert from the pACD4K vector, resulting in knockout of 7α-HSDH (sequencing data not shown).

### Reaction Example 1: Enzymatic Conversion of 7-Keto-LCA by 7β-HSDH

For verification of the biochemical function of 7β-HSDH, conversion of 7-keto-LCA by 7β-HSDH was carried out. The 20 ml reaction mixture contains 50 mM 7-keto-LCA (approx. 0.4 g), 5 U/ml 7β-HSDH and 0.05 mM NADP⁺. 4 U/ml ADH and 1% isopropanol were used for regeneration of NADPH (see Scheme 1). The reaction was carried out in a fume cupboard at pH8 and 24° C. with stirring. As acetone evaporates more quickly than isopropanol, the reaction is shifted toward formation of UDCA. Further 1% isopropanol was added after 24 h, 48 h and 72 h. The product was analyzed by TLC (silica gel 60, Merck, solvent petroleum ether and ethyl acetate 1:10, vol:vol). In TLC, the product was compared with authentic references 7-keto-LCA, UDCA and CDCA. The TLC analysis shows that UDCA was formed from 7-keto-LCA by the 7β-HSDH. The enantiomer CDCA is not detectable in TLC.

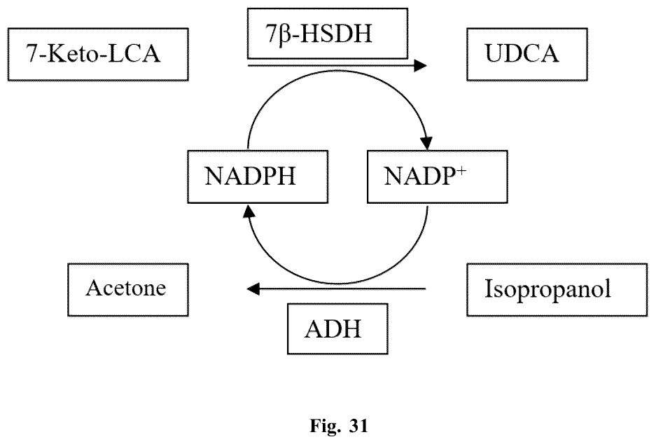

FIG. 31 shows a schematic representation of reduction of 7-keto-LCA by 7β-HSDH. The ADH regenerates the cofactor NADPH.

### Reaction Example 2: Enzymatic Production of 3,12-Diketo-7β-CA from DHCA by 76-HSDH

To verify the usability of 7β-HSDH for preparing 12-keto-UDCA from DHCA, conversion of DHCA by 7β-HSDH was carried out. The 50 ml of reaction mixture contain 50 mM DHCA (1 g), 5 U/ml 7β-HSDH and 0.05 mM NADP⁺. 4 U/ml ADH and 1% isopropanol were used for regeneration of NADPH (see Scheme 2). The reaction was carried out in a fume cupboard at pH8 and 24° C. with stirring. As acetone evaporates more quickly than isopropanol, the reaction is shifted toward formation of 3,12-diketo-7β-CA. In order to achieve complete conversion, further 1% isopropanol was added after 24 h, 48 h and 72 h. The intermediate 3,12-diketo-7β-CA was analyzed by TLC. The educt DHCA was no longer detectable in TLC (silica gel 60, Merck; solvent chloroform:methanol:acetic acid 10:1:0.08 vol:vol:vol).

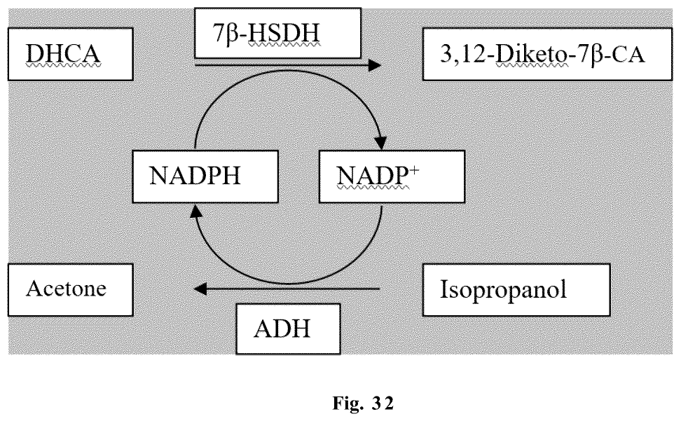

FIG. 32 shows a schematic representation of reduction of DHCA by 7β-HSDH. The ADH regenerates the cofactor NADPH.

### Reaction Example 3: Enzymatic Conversion of 3,12-Diketo-7β-CA to 12-Keto-UDCA

The intermediate 3,12-diketo-7β-CA (produced according to reaction example 2) was transformed further by 3α-HSDH (SEQ ID NO:5 and 6) from Comamonas testosteroni (Mobus, E. and E. Maser, Molecular Cloning, overexpression, and characterization of steroid-inducible 3alpha-hydroxysteroid dehydrogenase/carbonyl reductase from Comamonas testosteroni. A novel member of the short-chain dehydrogenase/reductase superfamily. J Biol Chem, 1998. 273(47): p. 30888-96) to 12-keto-UDCA. This 3α-HSDH requires cofactor NADH, which was regenerated by the FDH (see FIG. 3). 4U/ml 3α-HSDH, 1 U/ml FDH (NADH-dependent, Codexis), 200 mM sodium formate and 0.05 mM NAD⁺ were added to the reaction mixture. After 40 h the product was acidified with 2 M HCl to pH2 and extracted with 6×10 ml ethyl acetate. After evaporation, 1.07 g of product was obtained. The product 12-keto-UDCA was analyzed and confirmed by TLC and NMR. 3alpha-HSDH was prepared as for the preparation of 7β-HSDH, but with the plasmid pET22b+, and was used without further purification.

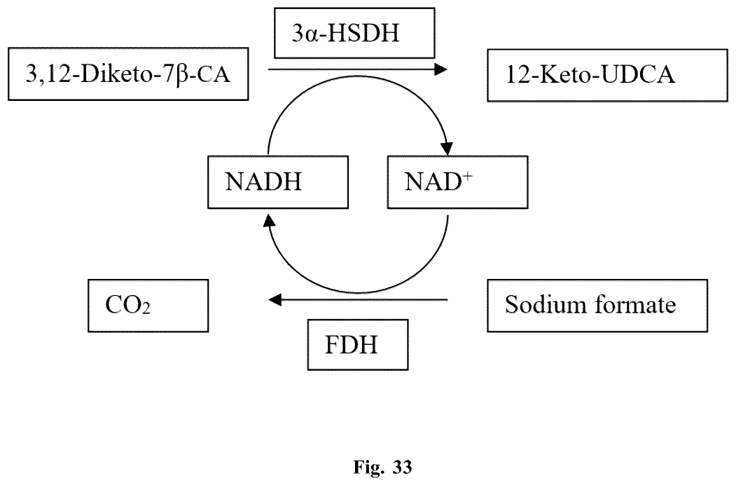

FIG. 33 shows a schematic representation of reduction of 3,12-diketo-7β-CA by 3α-HSDH. The FDH regenerates the cofactor NADH.

### Reaction Example 4: Chemical Reaction of CA to DHCA

1320 L of glacial acetic acid is put in a 2000 L stirred vessel and 110 kg (260 mol) of cholic acid (CA) is dissolved therein. 422 L of sodium hypochloride solution (2.3 molar) is added to this solution at 20 to 40° C. and the reaction solution is then stirred for at least a further 1 hour for completion of the reaction. Dehydrocholic acid (DHCA) is isolated by centrifugation at a yield of 100 kg (90%).

### Reaction Example 5: Chemical Reaction of 12-Keto-UDCA to UDCA

105 g (0.258 mol) of 12-keto-UDCA is dissolved in 384 ml triethylene glycol, 52.2 g (1.304 mol) sodium hydroxide and 75.95 ml (1.563 mol) hydrazine hydrate and heated slowly to 180° C. There is formation of hydrazone, which starting from 160° C. is transformed with splitting off of nitrogen to UDCA. The reaction mixture is held at 180° C. for 8 hours for completing the reaction. The reaction mixture is cooled to below 100° C., and 1500 ml water is added. Then the UDCA is precipitated by acidifying with hydrochloric acid. The product is obtained at a yield of 96.2 g to 99.2 g (up to 95%-98%).

### Reaction Example 6: Enzymatic Conversion of DHCA to 12-Keto-UDCA by 7β-HSDH, FHD D221G and 3α-HSDH

The purpose of this example is to investigate whether a two-step enzymatic conversion of DHCA to 12-keto-UDCA with simultaneous cofactor regeneration with an FDH mutant used according to the invention is possible. As the FDH mutant D221G used accepts both NADP⁺ and NAD⁺ as cofactor, for the NADH-dependent 3α-HSDH it is not necessary for the reaction mixture to contain any additional cofactor regeneration system.

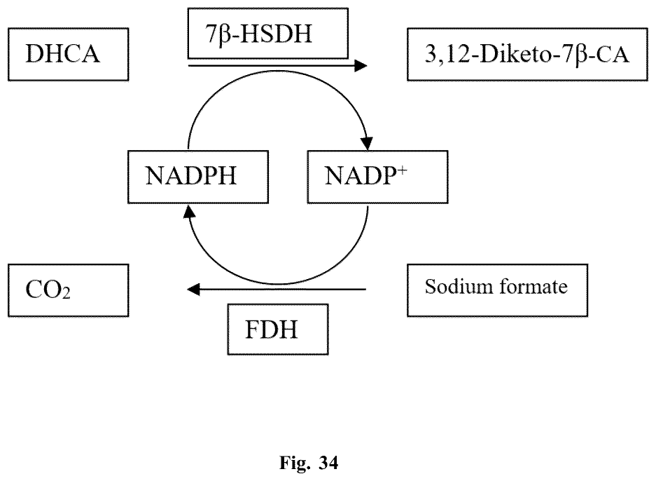

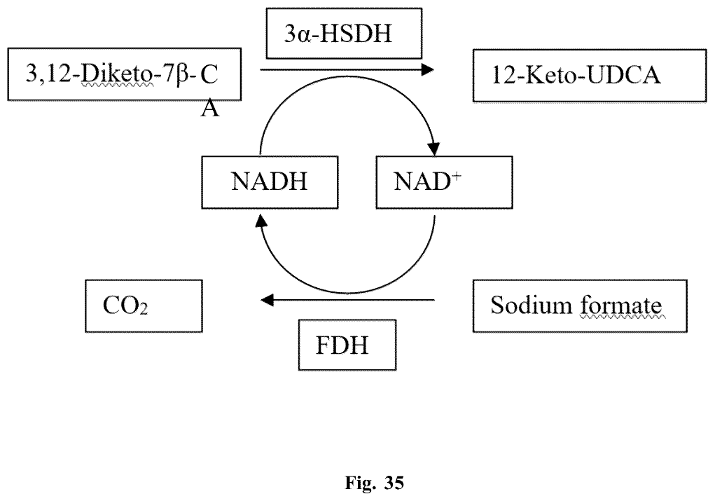

FIG. 34 and FIG. 35 show two partial reactions based on either NADPH or NADH. FDH* designates the mutant FDH D221G.

For this, the enzymes 7β-HSDH from Collinsella aerofaciens, 3α-HSDH from Comamonas testosteroni and the FDH mutant D221G derived from FDH from Mycobacterium vaccae were expressed separately from one another in a modified E. coli expression strain and were used for the reaction. The E. coli strain used for expression was modified so that it does not express any 7α-HSDH enzyme activity. This side activity, widely occurring in E. coli, can lead, in reactions of the present type (stereospecific conversion of the 7-keto group), to undesirable side reactions and therefore to contamination of the reaction product to be produced.

In the next experiment, the knock-out strain E. coli BL21(DE3)A7α-HSDH was used, which is described in the applicant's earlier European patent application EP 10164003.5. Reference is expressly made hereby to the disclosure of this patent application.

### 6.1 Plasmids Used

Plasmids for the expression of 7β-HSDH, 3α-HSDH and FHD D221G:
pET28a(+)-7β-HSDH,
pET22b(+)-3α-HSDH and
pET21a(+)-FDH-D221G.

### 6.2 Bacterial Strains and Culture Conditions:

The aforementioned knock-out strain E. coli BL21(DE3)A7α-HSDH was cultured at 37° C. in LB medium containing the necessary antibiotics. After induction with 0.5 mM IPTG on reaching OD₆₀₀=0.8, incubation was continued at 140 rpm for a period of 12 hours at 25° C.

### 6.3 Enzyme Overexpression and Purification

Overexpression of 7β-HSDH, 3α-HSDH and the FDH mutant D221G in E. coli BL21(DE3)Δ7α-HSDH and enzyme purification were carried out in the same conditions as described above in production example 2 for the overexpression and purification of 7β-HSDH in E. coli BL21 (DE3). The yields per liter of culture medium (shaken flask at OD₆₀₀˜6) were as follows:
7β-HSDH: 3883U (for DHCA and NADPH)
3α-HSDH: 6853 U (for DHCA and NADH)
FDH mutant: 47 U (for sodium formate and NAD⁺).
   Content and purity of the proteins were determined by SDS-PAGE and densitometer scanning using Scion Image Beta 4.0.2 (Scion, USA).

### 6.4 Preparative-Scale Enzymatic Synthesis of 12-Keto-UDCA

800 ml of a reaction mixture containing 7β-HSDH (2.4 U×ml-1), 3α-HSDH (2.4 U×ml-1), FDH D221G (0.325 U×ml-1), NADP⁺ (10 μM), NAD⁺ (10 μM), sodium formate (250 mM), DHCA (10 mM, 3.2 g) and potassium phosphate buffer (50 mM, pH 6) was stirred at 24° C. All three enzymes that were employed in this experiment were used as cell raw extracts without an additional purification step. After 12 hours the reaction was stopped by removing the enzymes by ultrafiltration using a membrane with a pore size of 10 kDa (Millipore, USA). The product in the filtrate was purified by acidifying with hydrochloric acid to pH 2 followed by paper filtration. After drying the product at 60° C. overnight, 2.9 g of the desired product was obtained.

The product was analyzed by HPLC and NMR.

### Analysis Data (Partial):

¹H NMR (deuterated DMSO, 500 MHZ) δ=3.92 (2H, m, H-3a and H-7β) and

¹H NMR (deuterated DMSO, 125 MHZ) δ=69.38 (CH, 3-C); δ=69.09 (CH, 7-C); δ=213.86 (C, 12-C)

Yield: 90.6%

Purity: 99%

### Reaction Example 7: Whole-Cell Biotransformation of DHCA to 12-Keto-UDCA by Coexpression of FDH D221G, 7β-HSDH and 3α-HSDH in the Two-Plasmid System

The aim was to investigate whether a two-step whole-cell reduction of dehydrocholic acid (DHCA) to 12-keto-ursodeoxycholic acid (12-keto-UDCA) (cf. scheme according to reaction example 6, above) is possible.

For this, the knockout strain E. coli BL21 (DE3) hdhA⁻KanR⁺ pET21a(+) FDH 7β-HSDH pCOLA(mod) 3α-HSDH prepared above was used, in which, in addition to the 7β-HSDH and the mutant FDH D221G, a 3α-HSDH from Comamonas testosteroni is expressed recombinantly. As the FDH mutant used accepts both NADP⁺ and NAD⁺ as cofactor, for the NADH-dependent 3α-HSDH it was not necessary to insert any additional cofactor regeneration system into the biotransformation strain.

### 7.1 Strain Used:

Escherichia coli BL21 (DE3) hdhA⁻KanR⁺

### 7.2 Molecular Biology Procedures Used

### 7.2.1 Polymerase Chain Reaction

The polymerase chain reaction (PCR) was used for cloning the 7β-HSDH. The plasmid pET22b(+) 7-HSDH served as template for amplification of the 7β-HSDH.

PCR reactions were carried out in 500 μL PCR tubes with 20 μL reaction volume. The reactions were carried out in a Thermocycler from the company Eppendorf. For amplification of 7β-HSDH, in each case 4 μL HF-buffer, 1 μL template DNA, 1 μL each of forward and reverse primer (10 μM), 0.4 μL of deoxynucleotide triphosphate solution (10 mM) and 0.2 μL of Phusion DNA polymerase (2 U/μL) were added. The volume of the preparation was adjusted to 20 μL with RNase-free water.

For the reaction, first a 2-min initial denaturation step was carried out at 98° C. Then 34 cycles of denaturation (30 s at 98° C.), primer hybridization (2 min at 48° C.) and elongation (2 min at 72° C.) were carried out. As a last step, final elongation of 10 min was carried out at 72° C. before stopping the polymerase chain reaction by cooling to 4° C.

### 7.2.2 Purification of DNA Fragments by Gel Extraction

For purification of DNA fragments, first they were separated by agarose gel electrophoresis. The corresponding bands were made visible with UV light, identified based on their size and cut out of the gel with a scalpel. Extraction was carried out with the QIAquick Gel Extraction Kit according to the manufacturer's protocol. 30-50 μL H₂O was used for elution of the purified DNA.

7.2.3 Restriction with Endonucleases

The restriction reactions were carried out in a total volume of 20-50 μL. For this, 10-20 U of the respective restriction enzymes was added to the DNA to be cut. In addition, the reaction buffer recommended by the manufacturer was used and optionally 0.5 μL of bovine serum albumin (BSA, 10 mg/mL) was added, based on the manufacturer's recommendation. Restriction digestion was carried out for 2 h at 37° C. Then the restricted fragments were purified either by gel extraction (vector digestion products) or using the QIAquick PCR Purification Kit (digested PCR products).

### 7.2.4 Purification of DNA Fragments Using the QIAquick PCR Purification Kit

In order to purify restricted PCR fragments, DNA was purified using the QIAquick PCR Purification Kit. Purification was carried out according to the manufacturer's instructions, using 30-50 μL H₂O for elution of the purified DNA.

### 7.2.5 Ligation of DNA Fragments

For cloning restricted DNA fragments into expression vectors, both DNA molecules were cut with the same restriction enzymes. By using two different enzymes, on the one hand relegation of the vector can be prevented, and on the other hand the insert can be incorporated in a defined orientation. The enzyme used was T4-DNA-ligase, which catalyzes the formation of a phosphodiester bond between a free 5′-phosphate group and a free 3′-OH end of a deoxyribonucleic acid.

For cloning the expression constructs, in each case 4 μL of a gene-coding, purified DNA fragment with restricted ends was added to 12 μL of a restricted, purified vector. Then 2 μL 10× ligase buffer, 1 μL adenosine triphosphate (ATP, 1 mM) and 1 μL T4-DNA-ligase (400 U/L) were added. The reactions continued overnight at 16° C.

### 7.3 Vector Constructs Used

7.3.1 pET21a(+) FDH 7β-HSDH

For this vector construct, 7β-HSDH was cloned into pET21a(+) FDH, so that the 7beta-HSDH coding gene is located downstream of the FDH. For this purpose, a stop codon had to be inserted in the FDH at the 5′-end. Compared with the original sequence (Lys-Lys-Ala-Val-stop), in this construct the C-terminal valine residue was replaced with an alanine residue and a further three amino acids were appended, resulting in the following C-terminal sequence: Lys-Lys-Ala-Ala-Gly-Asn-Ser-stop. Moreover, for increased translation, an additional ribosomal binding site was inserted in 7β-HSDH between FDH and 7beta-HSDH.

The primers 7beta_fwd_EcoRI and S_7 beta_rev_HindIII were used for PCR amplification of 7β-HSDH.

7beta_fwd_EcoRI (SEQ ID NO: 13):
5′-GCGAATTCGTGAAAGGAGATATACATGAACCTGAGGGAGAAGTACGG-3′
S 7beta_rev_HindIII (SEQ ID NO: 3):
5′-CCCAAGCTTCTAGTCGCGGTAGAACGA-3′

The hybridizing sequence regions (bold), restriction sites (bold underlined), ribosomal binding sites (underlined), and filling nucleotides (italics) are shown. In the primer 7beta_fwd_EcoRI, additionally a stop codon (double underlined) and a nucleotide for reading frame shift (bold) were added.
Cloning was carried out via the EcoRI and HindIII cleavage sites.
7.3.2 pCOLA(mod) 3α-HSDH

In order to be able to reduce dehydrocholic acid in two steps to 12-keto-ursodeoxycholic acid, in addition to the cofactor regeneration system FDH, the enzymes 7β-HSDH and 3α-HSDH must also be present in a cell. To make this possible, the vector construct pCOLA(mod) 3α-HSDH, a modified derivative of the pCOLA-duet vector, was prepared, which is cotransformable with pET-vectors.

For this purpose, the 3α-HSDH coding gene was cut out via the NdeI and BlpI cleavage sites from the vector pET22b(+) 3α-HSDH and, via the same cleavage sites, cloned into MCS2 of the pCOLA(mod) vector. After sequencing, it was found that at position 45 of the DNA sequence, a guanine was replaced with a cytosine, but this results in a silent mutation. However, this mutation has no effect on the amino acid sequence; therefore this is a so-called silent mutation.

Both vectors were cotransformed into the aforementioned strain, as described below. The genes are IPTG-inducible.

7.4 Heat-Shock Transformation of E. coli Cells

For this purpose, 200 μL of chemically-competent E. coli BL21 (DE3) hdhA⁻KanR⁺ or DH5a were thawed and 1 μL DNA was added. These were first incubated on ice for 45 min. Then the cells were submitted to heat shock for 45 s at 42° C. Then 600 μL of LB medium was added to the cells and they were shaken at 37° C. and 200 rpm, so that they could develop the desired antibiotic resistance. Next the cells were centrifuged in a benchtop centrifuge at 3000 rpm for 2 min, after which 150 μL of the supernatant was discarded. The cells were resuspended in the remaining supernatant and plated out on LB-agar plates with the corresponding antibiotic. Then the plates were incubated overnight at 37° C.

In the case of cotransformation of two different plasmids, as is necessary for creating the strain that contains the plasmids pCOLA(mod) 3α-HSDH and pET21a(+) FDH 7β-HSDH, in each case 2 μL of the plasmids to be inserted was added to 50 μL of chemically-competent E. coli BL21 (DE3) hdhA⁻KanR⁺. After transformation, the cells were added to preculture tubes with 5 mL of LB medium with the corresponding antibiotic and were incubated at 37° C. on the preculture shaker for 16-48 h at 200 rpm, until growth of bacteria in the preculture tube was detected. Next, 5 μL of the bacterial suspension was plated out on LB-agar plates with the corresponding antibiotic and was incubated overnight at 37° C.
7.5 Cultivation of the strain in the shaken flask:

For expression of recombinant proteins, the corresponding E. coli BL21 (DE3) hdhA⁻KanR⁺ comprising the two expression plasmids was incubated overnight at 37° C. and 200 rpm in 5 mL of LB medium with addition of the corresponding antibiotic. Then 200 ml of TB medium with addition of the corresponding antibiotic was inoculated with this preculture and incubated at 37° C., 250 rpm. On reaching OD600 of 0.6-0.8, expression of the recombinant protein was induced with 1 mM isopropyl-β-D-thiogalactopyranoside (IPTG) and the culture was incubated at 25° C., 160 rpm for a further 21 h.

### 7.6 Carrying Out Whole-Cell Biotransformation and Test Result

The reactions were carried out in suspension in 2 mL reaction volume at pH 6.0, using a cell density of OD₆₀₀=20. 25 mM or 50 mM dehydrocholic acid was selected as the substrate concentration, and the cosubstrate concentration used was 400 mM. The tests were carried out at 25° C. with exclusion of air. Samples were taken after 48 h.

In the case of the reaction mixture with 25 mM, substrate is no longer detectable, whereas for the reaction mixture with 50 mM dehydrocholic acid, a substrate peak is detectable, but its peak area is below the limit of 1 μg/mL substrate in the sample for which the HPLC method is calibrated. The same applies to the peaks of 3,12-diketo-ursodeoxycholic acid. The largest peak in the chromatograms is that of the product 12-keto-ursodeoxycholic acid.

Thus, it can be shown, surprisingly, that a two-step whole-cell reduction of dehydrocholic acid to 3-keto-ursodeoxycholic acid is possible.

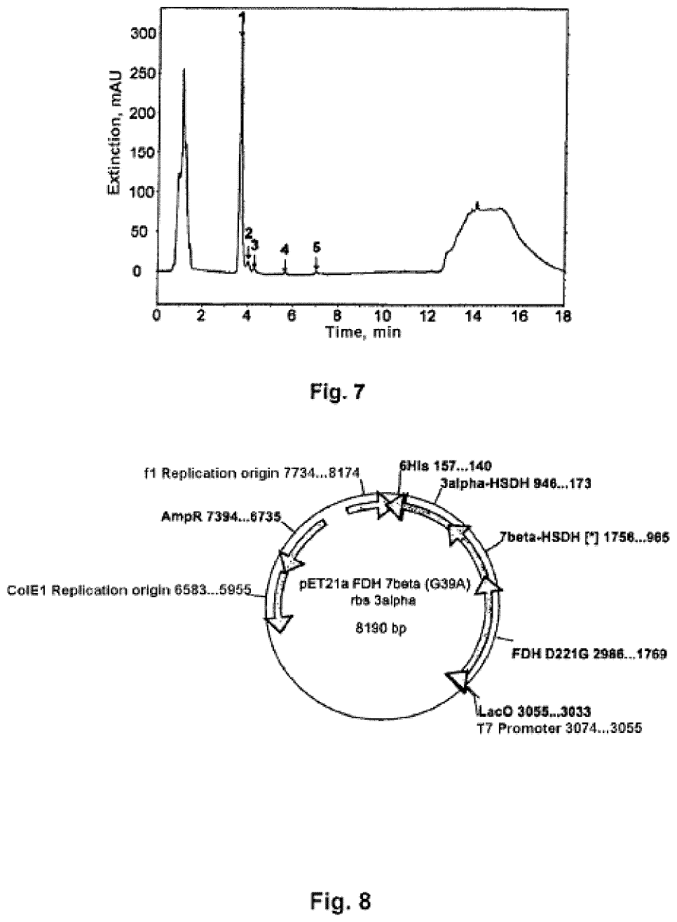

The chromatograms of the HPLC measurement are shown in FIG. 7.

The HPLC analysis was carried out as follows: The chromatography column used was the reverse-phase chromatography column Hi-bar Purospher 125-4 RP-18e (5 μm) from the company Merck, Darmstadt. In this case a nonpolar phase serves as stationary phase, whereas a polar phase forms the mobile phase. The method of gradient elution was used for the HPLC analysis. An increasing proportion of acetonitrile is added to the eluent, phosphoric acid water, (pH 2.6), and the acidification of the solvent causes a uniform degree of protonation of the analytes to be investigated. After elution of all components, the chromatography column is equilibrated with the starting ratio of the two solvents. Gradient elution was carried out by first pumping a solvent mixture with a constant composition of 65% (v/v) of phosphoric acid water and 35% (v/v) of acetonitrile through the HPLC column for 3 min. From minute 3 to 7.5, the proportion of acetonitrile is increased linearly to 39% (v/v). Between minute 7.5 and 10 there is another linear increase of the proportion of acetonitrile to 40% (v/v). For elution of all sample components, the proportion of acetonitrile is increased between minute 10 and 11 also linearly to 70% (v/v) and held constantly at this value for a further two minutes. After that, the proportion of acetonitrile is reduced from minute 13 to 15 back to the initial value of 35% and the column is equilibrated with this solvent composition for 3 min. The flow is set at 1.000 mL/min for the total duration. Eluted analytes are detected by a UV-detector at a wavelength of 200 nm.

### Reaction Example 8: Whole-Cell Biotransformation of DHCA to 12-Keto-UDCA by Coexpression of FDH D221G, 76-HSDH and 3α-HSDH in the Single-Plasmid System

The aim was to investigate whether a two-step whole-cell reduction of dehydrocholic acid to 12-keto-ursodeoxycholic acid in a cellular single-plasmid system is possible.

### 8.1 Plasmids Used:

| | | SEQ |
|---|---|---|
| | | ID |
| Designation | Sequence | NO |
| 3alpha_fwd_ | 5′-CCCAAGCTTAAGGAGATATACATGTCCATCA | 16 |
| HindIII | TCGTGATAAGCG-3′ | |
| 3alpha_rev_ | 5′-ATAAGAATGCGGCCGCTCAGAACTGTGTCGG | 17 |
| NotI | GCG-3′ | |
| 7beta_mut_ | 5′-CGTCGTCATGGTCGCCCGTCGCGAGG-3 | 9 |
| G39A_fwd | | |
| 7beta_mut_ | 5′-CCTCGCGACGGGCGACCATGACGACG-3′ | 10 |
| G39A_rev | | |

Restriction sites are underlined; ribosomal binding sites are bold.

### 8.2 Production of the Vector:

The plasmid construct pET21a(+) FDH 7β-HSDH(G39A) 3α-HSDH (FIG. 8) is prepared as follows: Starting from the plasmid pET21a(+) FDH 7β-HSDH the wild-type 3α-HSDH (C. testosteroni; SEQ ID NO: 5,6) was cloned in after 7β-HSDH via the HindIII and NotI cleavage sites. For amplification of 3α-HSDH, the primers 3alpha_fwd_HindIII and 3alpha_rev_NotI were used, with the plasmid pET22b(+) 3α-HSDH serving as template for this. Then the G39A mutation was inserted into 7β-HSDH by site-directed mutagenesis according to the QuikChange protocol. The mutagenesis primers 7beta_mut_G39A_fwd and 7beta_mut_G39A_rev were used.

### 8.3 Reaction:

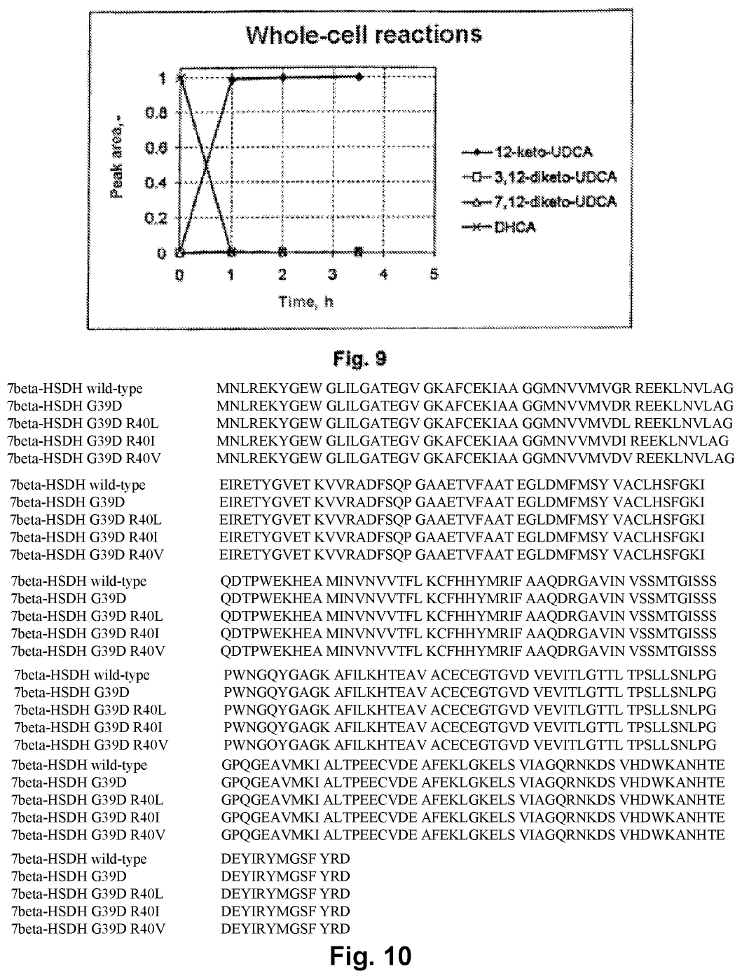

The plasmid prepared was transformed into E. coli BL21 (DE3) hdhA⁻KanR⁺. With the resultant strain E. coli BL21 (DE3) hdhA⁻KanR⁺ pET21a(+) FDH 7β-HSDH(G39A) 3α-HSDH, whole-cell reactions were carried out under the following conditions at the 150-mL scale: cell density OD 30, 50 mM DHCA, 750 mM sodium formate, suspended in 50 mM potassium phosphate buffer (pH 6.5) as cell and substrate suspension. The reactions were carried out for 3.5 h at 25° C. The results of the HPLC analysis (for procedure see reaction example 7, above) are shown in FIG. 9.

### Production Example 6: Production of Further 7β-HSDS Mutants and Characterization Thereof

One possibility for generating an NADH-specific 7β-HSDH consists of modifying the available enzyme by various techniques of mutagenesis. Therefore, using site-directed mutagenesis, individual amino acids of 7β-HSDH were to be substituted with others, which cause change of the cofactor specificity of 7β-HSDH from NADPH to NADH, so that advantageously NADP can be replaced with the less expensive NAD.

### 6.1 Primers

Altogether, the 7β-HSDH mutants G39D, G39D/R40L, G39D/R40I and G39D/R40V were produced. The G39D mutant is produced by Quickchange mutagenesis, and the other mutants are produced by the PCR method of Sanchis et al. (Sanchis J, Fernández L, Carballeira J D, Drone J, Gumulya Y, Höbenreich H, Kahakeaw D, Kille S, Lohmer R, Peyralans J J, Podtetenieff J, Prasad S, Soni P, Taglieber A, Wu S, Zilly F E, Reetz M T. Improved PCR method for the creation of saturation mutagenesis libraries in directed evolution: application to difficult-to-amplify templates. Appl Microbiol Biotechnol. 2008 November; 81(2): 387-97). A sequence comparison of the wild type and of the mutants produced is shown in FIG. 10. The following primers were used for the mutagenesis, with the bases shown in italics coding in each case for the exchanged amino acid:

G39D
7beta mut G39D fwd (SEQ ID NO: 41):
CGTCGTCATGGTCGACCGTCGCGAGG
7beta mut G39D rev (SEQ ID NO: 42):
CCTCGCGACGGTCGACCATGACGACG
G39D R40L
7beta mut G39D R40L fwd (SEQ ID NO: 43):
CGTCGTCATGGTCGACCTGCGCGAGG
3alphamut_AntiMid_rev (SEQ ID NO: 44):
CCGCCGCATCCATACCGCCAGTTGTTTACCC
G39D R40I
7beta mut G39D R40I fwd (SEQ ID NO: 45):
CGTCGTCATGGTCGACATTCGCGAGG
3alphamut_AntiMid_rev (SEQ ID NO: 44):
CCGCCGCATCCATACCGCCAGTTGTTTACCC
G39D R40V
7beta mut G39D R40V fwd (SEQ ID NO: 46):
CGTCGTCATGGTCGACGTTCGCGAGG
3alphamut_AntiMid_rev (SEQ ID NO: 44):
CCGCCGCATCCATACCGCCAGTTGTTTACCC

### 6.2 Enzyme-Kinetic Investigation of the 76-HSDH Mutants

The mutants prepared are evaluated by means of enzyme-kinetic investigations, the results of which are shown as graphs in FIG. 11. 0.1 mM NADPH is used as cofactor for investigating the unmodified 7β-HSDH, but 0.5 mM NADH is used for investigating the 7β-HSDH mutants. The need to increase the cofactor concentration in the enzyme-kinetic investigation of the 7β-HSDH mutants is due to the increased semisaturation concentrations for the cofactor NADH in the mutants. From the plots of substrate concentration versus specific enzyme activity, the characteristic curves of Michaelis-Menten kinetics can be seen for the 7β-HSDH mutants, whereas the curve of Michaelis-Menten kinetics with substrate inhibition can be seen from the plot for the unmodified wild-type 7β-HSDH. Accordingly, the classical Michaelis-Menten model (equation 1) was used for evaluating the measurement series of the 7β-HSDH mutants and the Michaelis-Menten model with substrate inhibition was used for the measurement series of the unmodified wild-type 7-HSDH (equation 2).

$$\begin{matrix}Equation 1 ( Michaelis - Menten equation ) & \\ {EA}_{X} = {v}_{max} \text{?} \frac{{c}_{\text{?}}}{{K}_{m} + {c}_{\text{?}}} & \end{matrix}$$

$$\text{?} \text{indicates text missing or illegible when filed}$$

EAₓ: specific enzyme activity, U mg⁻¹=μmol min⁻¹ mg⁻¹
vₘₐₓ: maximum specific enzyme activity, U mg⁻¹=μmol min⁻¹ mg⁻¹
cₛ: substrate or cofactor concentration, mol L⁻¹
Kₘ: semisaturation concentration, mol L⁻¹

$$\begin{matrix}Equation 2 ( Michaelis - Menten equation with substrate inhibition ) & \\ {EA}_{X} = v \text{?} \cdot \frac{c \text{?}}{K \text{?} + ( 1 + \frac{( c \text{?} )}{( {K}_{i} )} ) c \text{?}} & \end{matrix}$$

$$\text{?} \text{indicates text missing or illegible when filed}$$

EAₓ: specific enzyme activity, U mg⁻¹=μmol min⁻¹ mg⁻¹
vₘₐₓ: maximum specific enzyme activity, U mg⁻¹=μmol min⁻¹ mg⁻¹
cₛ: substrate or cofactor concentration, mol L⁻¹
Kₘ: semisaturation concentration, mol L⁻¹
Kᵢ: inhibition constant, mol L⁻¹
   The following table gives enzyme-kinetic parameters of the unmodified wild-type 7β-HSDH and the mutants thereof with altered cofactor specificity. NADPH is used as cofactor for the wild type, whereas NADH is used as cofactor for the mutants.

| | Kₘ, | Kᵢ, | vₘₐₓ, |
|---|---|---|---|
| | DHCA, μM | DHCA, μM | U mg − 1 |
| Wild type | 31 ± 5 | 8600 ± 1500 | 14.6 ± 1.0 |
| G39D | 660 ± 120 | n.d. | 2.90 ± 0.16 |
| G39D R40I | 920 ± 170 | n.d. | 4.64 ± 0.27 |
| G39D R40V | 880 ± 20 | n.d. | 2.69 ± 0.12 |
| G39D R40L | 560 ± 80 | n.d. | 1.60 ± 0.07 |

### Production Example 7: Production of Further 76-HSDS Mutants and Characterization Thereof

Further 7β-HSDS mutants were produced in a preparation parallel to production example 6.

### 7.1 Primers

The mutagenesis primers shown below were used for the site-directed mutagenesis of 7β-HSDH. The primers were selected on the basis of the 7β-HSDH gene sequence, so that they bring about the desired amino acid exchange. It was noted that the base to be mutated is localized centrally in the primer, and that the melting points of the primer pairs are located in the same region.

The following primer pairs were used for preparing the mutants:

TABLE
Primers used for the site-directed mutagenesis of 7β-HSDH

| Designation | Position | Primer | 5′ -> 3′ Sequence |
|---|---|---|---|
| G39D_for | G39D | forward | GTCGTCATGGTCGACCGTCGCGAGGAG |
| G39D_rev | | reverse | CTCCTCGCGACGGTCGACCATGACGAC |
| G39D/R40I_for | G39D/R40I | forward | GTCATGGTCGACATTCGCGAGGAG |
| G39D/R40I_rev | | reverse | CTCCTCGCGAATGTCGACCATGAC |
| R40D_for | R40I | forward | GTCATGGTCGGCGATCGCGAGGAGAAG |
| R40D_rev | | reverse | CTTCTCCTCGCGATCGCCGACCATGAC |
| R40D/R41I_for | R40D/R41I | forward | GTCATGGTCGGCGATATCGAGGAGAAGCTG |
| R40D/R41I_rev | | reverse | CAGCTTCTCCTCGATATCGCCGACCATGAC |
| DIN_for | G39D/R40I/R41N | forward | ATGGTCGACATTAACGAGGAGAAGCTG |
| DIN_rev | | reverse | CAGCTTCTCCTCGTTAATGTCGACCAT |

### 7.2 PCR Program

In the reaction, first there was a 2-min initial denaturation step at 98° C. This was followed by 23 cycles of denaturation (30 s at 98° C.), primer hybridization (1 min at 60-68° C.) and elongation (3.5 min at 72° C.). As the last step, a final elongation was carried out for 10 min at 72° C. before the polymerase chain reaction was stopped by cooling to 4° C.

### 7.3 PCR Assay

TABLE
PCR assay for the production of the
different 7β-HSDH variants

| | |
|---|---|
| HF buffer (5×) | 10 μl |
| dNTP mix (10 mM) | 1.0 μl |
| Forward primer (10 pmol/μl) | 5 μl |
| Reverse primer (10 pmol/μl) | 5 μl |
| Template | 1.5 μl |
| Phusion polymerase | 0.5 μl |
| DMSO | 2.5 μl |
| ddH₂O | 24.5 μl |
| | 50 μl |

A pET21a vector with the 7β-HSDH (wild type) was used as template.

### 7.4 Procedure

For targeted exchange of amino acids in protein sequences, the DNA sequence of the corresponding gene is submitted to site-directed mutation. For this, mutually complementary primers are used, which carry the desired mutation in their sequence. N6-adenine-methylated, double-stranded plasmid DNA, which carries the gene to be mutated, serves as template. N6-adenine-methylated plasmid DNA is isolated from dam⁺ E. coli strain, for example E. coli DH5.

The polymerase chain reaction is carried out as described above. The primers are lengthened complementarily to the template, so that plasmids with the desired mutation are formed, which have a strand break. In contrast to other PCR reactions, the increase in DNA yield is in this case only linear, as newly formed DNA molecules cannot serve as template for the PCR reaction.

On completion of the PCR reaction, the PCR product was purified using a PCR-Purification-Kit (Analytik Jena) and the parental, N6-adenine-methylated DNA was digested with the restriction enzyme DpnI. This enzyme has the particular characteristic that it restricts N6-adenine-methylated DNA nonspecifically, but not the newly formed, nonmethylated DNA. Restriction was carried out by adding 1 μL DpnI to the PCR reaction mixture and incubating for 2 h or overnight at 37° C.

5 μl of this preparation were used for the transformation of 100 μl of chemically-competent DH5a cells. After plasmid isolation, successful mutation was confirmed by sequencing.

### 7.5 Characterization

The mutation was introduced into 7β-HSDH by site-directed mutagenesis according to the QuikChange protocol. The mutagenesis primers from the table shown in Section 7.1 were used.

Using the assays given in the “Methods” section for photometric measurement of activity, the activity of the mutated enzymes was measured in the presence of NADPH or NADH. The activities found are presented in the following table.

TABLE
Activity found for the different 7β-HSDH variants

| | Volumetric activity | | Specific activity | |
|---|---|---|---|---|
| Mutants | [U/ml] | | [U/mg] | |
| produced | NADPH | NADH | NADPH | NADH |
| G39D | 119 | 2 | 5.1 | 0.1 |
| G39D/R40I | 0 | 21 | 0 | 0.8 |
| R40D | 0 | 3 | 0 | 0.1 |
| R40D/R41I | 0 | 3 | 0 | 0.2 |
| G39D/R40I/R41N | 0 | 6 | 0 | 0.3 |
| 7β-HSDH (WT) | 134 | 0 | 5.6 | 0 |

### Reaction Example 9: Using NADH-Dependent 7β-HSDH in the Whole-Cell Reduction of Dehydrocholic Acid to 3,12-Diketo-UDCA

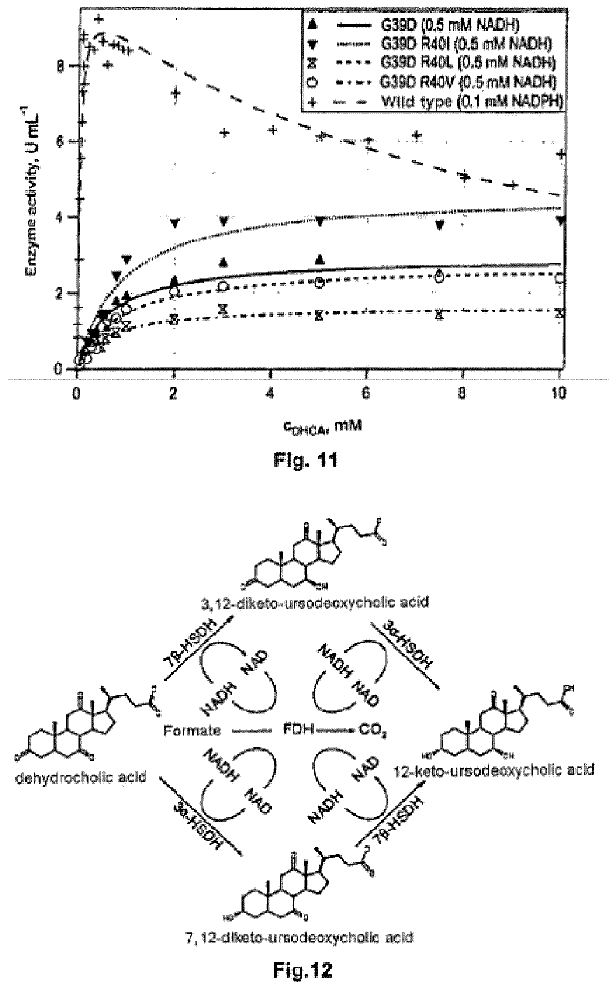

The use of NADH-dependent 7β-HSDH, produced in production example 6, in the whole-cell biocatalytic conversion of DHCA to 3,12-diketo-UDCA is demonstrated below. To improve comprehension, a review of possible reaction pathways and reaction products is shown in FIG. 12.

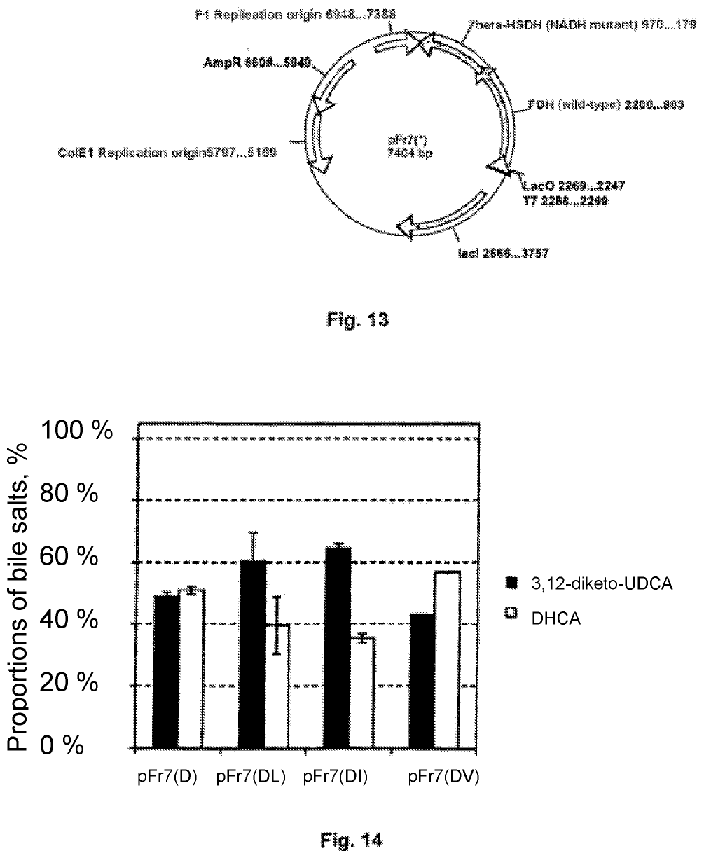

In the present case, the 7β-HSDH mutants together with an NADH-dependent formate dehydrogenase from Mycobacterium vaccae were inserted in an expression vector. The vector into which 7β-HSDH (G39D) is inserted bears the designation pFr7(D), corresponding to SEQ ID NO:49; the vector into which 7β-HSDH (G39D R40L) is inserted bears the designation pFr7(DL), corresponding to SEQ ID NO:50; the vector into which 7β-HSDH (G39D R40I) is inserted bears the designation pFr7(DI), corresponding to SEQ ID NO:51, and the vector into which 7β-HSDH (G39D R40V) is inserted bears the designation pFr7(DV), corresponding to SEQ ID NO:52. A general plasmid map of these vectors is shown in FIG. 13.

These vectors were transformed into the strain E. coli BL49 (identical to E. coli BL21(DE3) hdhA⁻KanR⁺). From these strains, firstly 5 mL overnight cultures were grown in LB medium with 100 mg L⁻¹ ampicillin. In each case 1 mL of these overnight cultures was transferred on the next day into 200 mL of TB medium in shaken flasks with 100 mg L⁻¹ ampicillin. These cultures were cultured at 37° C. and 250 rpm in the shaking incubator, until an OD of 0.6-0.8 was reached. Then induction was carried out with 1 mM isopropyl-β-D-thiogalactopyranoside (IPTG). The subsequent expression phase was carried out for 21 h at 25° C. and 160 rpm. The cells were harvested by 10-minute centrifugation at 3220 g.

Whole-cell biotransformation reactions were set up with the cells produced in this way. The concentrations of the substrate DHCA and of the product 3,12-diketo-UDCA in the reaction mixture at various time points was determined by high-performance liquid chromatography (HPLC).

The reaction mixtures for the biotransformations contained 17.7 g L⁻¹_{BTM} whole-cell biocatalysts, 100 mM substrate (DHCA), 500 mM sodium formate and are suspended in 50 mM potassium formate buffer (pH 6.5). The process took place at the 20 mL scale as a batch process without pH monitoring. The concentrations of product and substrate after 24 h are shown in FIG. 14. It can be seen that all mutants are suitable for the whole-cell biocatalytic conversion of DHCA to 3,12-diketo-UDCA.

### Reaction Example 10: Using NADH-Dependent 76-HSDH in the Two-Step Whole-Cell Reduction of Dehydrocholic Acid to 12-Keto-UDCA

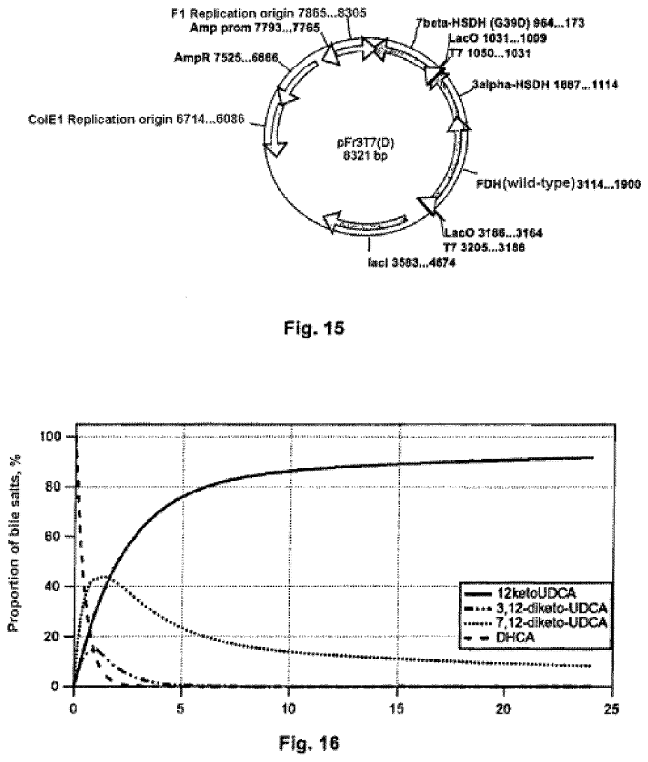

The use of NADH-dependent 7β-HSDH in the two-step whole-cell biocatalytic conversion of DHCA to 12-diketo-UDCA is to be demonstrated below. The mutants 7β-HSDH (G39D) together with an NADH-dependent formate dehydrogenase from Mycobacterium vaccae and an NADH-dependent 3α-HSDH from Comamonas testosteroni were inserted into an expression vector. The use of 7β-HSDH (G39D) is shown as an example for all NADH-dependent 7β-HSDHs. The vector bears the designation pFr3T7(D) and is shown schematically in FIG. 15.

These vectors were transformed into the strain E. coli BL49 (identical to E. coli BL21(DE3) hdhA⁻KanR⁺); the resultant strain bears the designation E. coli BL49 pFr3T7(D). This strain was cultured in the 7 L bioreactor at a working volume of 4 L in the defined minimal medium. A brief initial phase at 37° C. was followed by a substrate-limited exponential growth phase at 30° C. On reaching an optical density of 25-30, protein expression of the cells was induced by adding 0.5 mM IPTG. Expression was then carried out for 24 h at 20° C. The cells (32.0 g L⁻¹_{BTM}) were harvested by centrifugation, resuspended in potassium phosphate buffer (pH 6.5) and stored according to the standard protocol at −20° C.

Biotransformation was set up at the 20-mL scale in the following reaction conditions: 100 mM DHCA, 17.7 g L⁻¹_{BTM} of the stored biocatalyst, 500 mM ammonium formate, 26% glycerol, 50 mM MgCl₂, suspended in 50 mM KPi buffer (pH 6.5). Using manual pH adjustment, the pH was maintained during the first 5 hours of the biotransformation process at pH 6.5. The bile salt concentrations are shown as a function of time in FIG. 16. After 24 h, 92% of the product 12-keto-UDCA had formed. The conversion of DHCA to 3,12-diketo-UDCA and the conversion of 7,12-diketo-UDCA to 12-keto-UDCA show that all the reactions of 7β-HSDH shown in FIG. 12 are in fact catalyzed by 7β-HSDH (G39D).

### Reaction Example 11: Two-Step Reduction of Dehydrocholic Acid in a Single-Cell System

One route for the synthesis of ursodesoxycholic acid comprises the chemical oxidation of cholic acid to dehydrocholic acid with subsequent asymmetric enzymatic reductions of the 3-carbonyl group to the 3α-hydroxyl group and of the 7-carbonyl group to the 7β-hydroxyl group followed by chemical removal of the 12-carbonyl group.

For this synthesis route, the enzymes 7β-hydroxysteroid dehydrogenase (7β-HSDH) and 3α-hydroxysteroid dehydrogenase (3α-HSDH) are required for catalysis of the enzymatic reduction reactions. In the reaction, in addition cofactors (NADH or NADPH) are consumed stoichiometrically, and in an economic process these must be regenerated. Enzymatic regeneration possibilities are often preferred for this. Suitable enzymes include formate dehydrogenases (FDH), glucose dehydrogenases (GDH), glucose-6-phosphate dehydrogenases (G6PDH), alcohol dehydrogenases (ADH) or phosphite dehydrogenases (PtDH).

In contrast to the use of isolated enzymes, in whole-cell biocatalysis, with the necessary enzymes within a host organism, typically a unicellular microorganism, it is not possible to add individual enzymes in individual amounts to the reaction medium. In this case the enzyme activities must be balanced by modifying the expression level of all participating enzymes either by cultivation methods or by genetic modification of the whole-cell biocatalyst.

In the present example, vectors were used according to the invention, which contain all three genes for 7β-HSDH, 3α-HSDH and FDH. The vectors are constructed so that the expression level of the three genes in the designated host strain, especially whole-cell biocatalyst strains based on Escherichia coli BL21(DE3), are adapted in such a way that all three enzymes have enzyme activities that are as similar as possible.

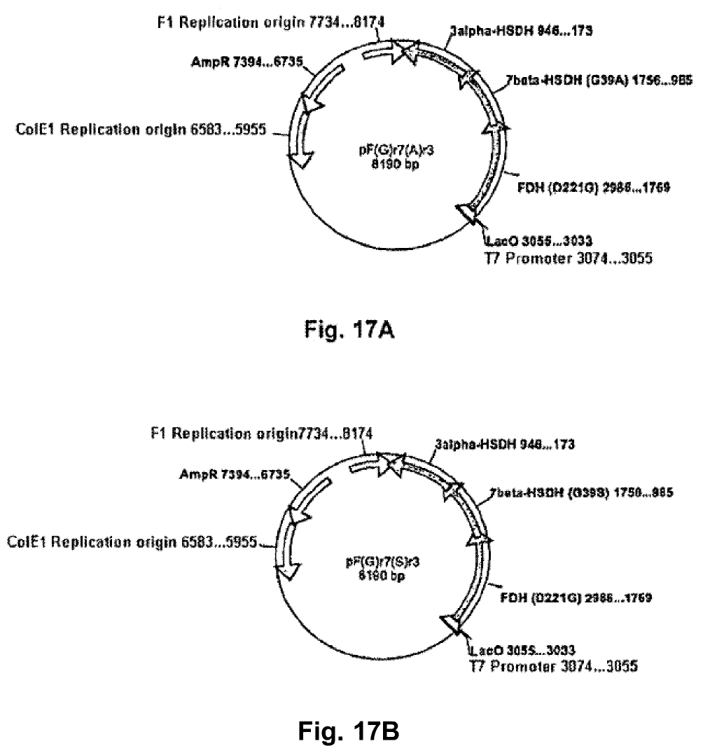

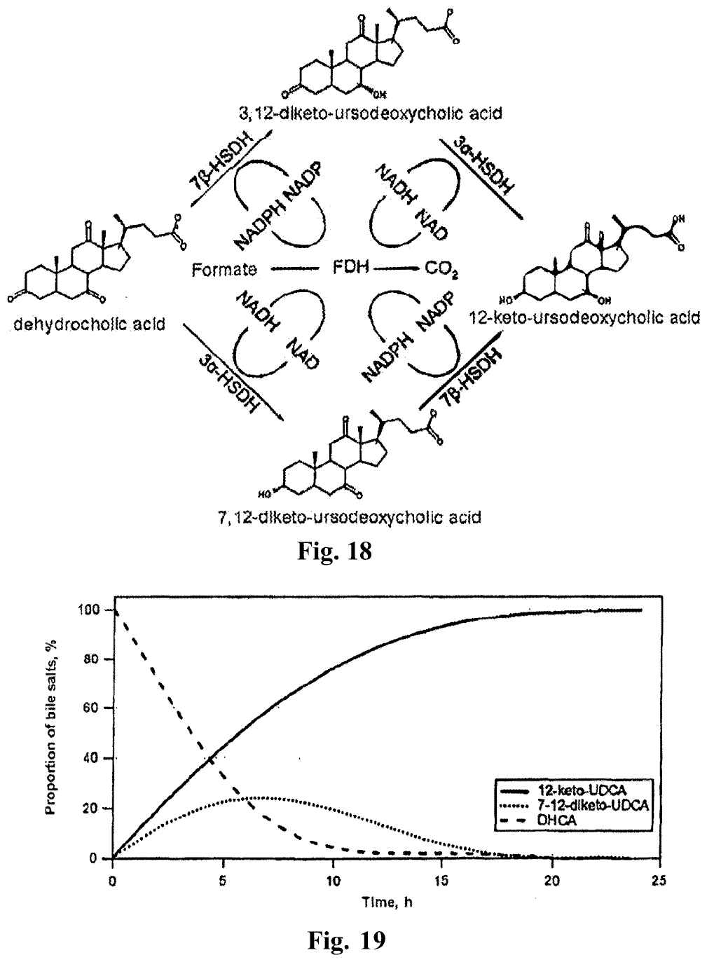

The three genes required in the whole-cell reduction of dehydrocholic acid to 12-keto-ursodeoxycholic acid are located on one and the same vector. These are genes that code for the following enzymes: an NADPH-dependent 7β-HSDH from Collinsella aerofaciens, an NADH-dependent 3α-HSDH from Comamonas testosteroni, a mutated FDH from Mycobacterium vaccae that is both NAD- and NADP-dependent. The expression levels of these three genes are balanced by the genetic construct, so that optimal whole-cell biocatalysis can be carried out. In the present example the 7β-HSDH mutants G39A and G39S were inserted instead of an unmodified wild-type enzyme into a vector for the whole-cell biocatalysis. The vectors bear the designations pF(G)r7(A)r3 and pF(G)r7(S)r3 and are shown in FIGS. 17a and b. FIG. 18 shows a schematic representation of the possible reaction pathways and reaction products.

These vectors according to the invention were transformed into Escherichia coli production strains. These strains represent the whole-cell biocatalyst. The host strain used is a modified E. coli BL21(DE3) with the designation E. coli BL49. However, the host organism is not restricted to this host strain. The E. coli BL49 transformed with pF(G)r7(A)r3 bears the designation E. coli BL49 pF(G)r7(A)r3, and the E. coli BL49 transformed with pF(G)r7(S)r3 bears the designation E. coli BL49 pF(G)r7(S)r3.

### 11.1 Two-Step Whole-Cell Reduction of Dehydrocholic Acid at the 20-mL Scale

The biocatalysts were compared by whole-cell biotransformation at the 20-mL scale. For this, first a 5 mL overnight culture was grown in LB medium with 100 mg L-1 ampicillin. On the next day, 1 mL of this overnight culture was transferred into 200 mL of TB medium in a shaken flask with 100 mg of L-1 ampicillin. These cultures were cultured at 37° C. and 250 rpm in the shaking incubator, until an OD of 0.6-0.8 was reached. Then induction was carried out with 1 mM isopropyl-β-D-thiogalactopyranoside (IPTG). The subsequent expression phase was carried out for 21 h at 25° C. and 160 rpm. The cells were harvested by 10-minute centrifugation at 3220 g.

Whole-cell biotransformation reactions were set up with the cells produced in this way. The amount of the product (12-keto-UDCA) formed in the reaction mixture was decisive for assessment of the cells. The concentrations of the substrate DHCA, of the intermediates 3,12-diketo-UDCA and 7,12-diketo-UDCA and of the product 12-keto-UDCA in the reaction mixture were determined by high-performance liquid chromatography (HPLC).

The reaction mixtures for the biotransformations contained 11.8 g L⁻¹_{BTM} whole-cell biocatalysts, 100 mM substrate (DHCA), 500 mM sodium formate and were suspended in 50 mM potassium formate buffer (pH 6.5). The process was carried out at the 20 mL scale as a batch process without pH monitoring. The following table gives the results of the whole-cell biotransformation described above after a process time of 5 h. The two new strains E. coli BL49 pF(G)r7(S)r3 and E. coli BL49 pF(G)r7(A)r3 are compared with a reference strain E. coli BL49 pF(G)r7 pC3. With the reference strain E. coli BL49 pF(G)r7 pC3 only 5.7±1.0% of product (12-keto-UDCA) was formed, whereas with the new biocatalysts 38.4±8.2% 12-keto-UDCA was formed with the strain E. coli BL49 pF(G)r7(S)r3 and 47.7±2.1% 12-keto-UDCA with the strain E. coli BL49 pF(G)r7(A)r3. Therefore the product concentration in the reaction mixture had increased relative to the reference strain by a factor of 6.7 (E. coli BL49 pF(G)r7(S)r3) and by a factor of 8.4 (E. coli BL49 pF(G)r7(A)r3).

Accordingly, the following table shows proportions of bile salts in biotransformation batches after 5 h when using 100 mM substrate (DHCA). The batches with the two new biocatalyst strains E. coli BL49 pF(G)r7(A)r3 and E. coli BL49 pF(G)r7(S)r3 are shown in comparison with the strain E. coli BL49 pF(G)r7 pC3 according to the prior art:

| | Proportions of bile salts, % | | | |
|---|---|---|---|---|
| | 12-keto- | 3,12-diketo- | 7,12-diketo- | |
| | UDCA | UDCA | UDCA | DHCA |
| E. coli BL49 | 5.7 ± 1.0% | 9.2 ± 0.2% | 40.5 ± 1.9% | 44.6 ± 3.1% |
| pF(G)r7 pC3 | | | | |
| E. coli BL49 | 47.7 ± 2.1% | 2.4 ± 0.5% | 48.9 ± 1.6% | 1.0 ± 0.0% |
| pF(G)r7(A)r3 | | | | |
| E. coli BL49 | 38.4 ± 8.2% | 2.7 ± 2.7% | 57.4 ± 4.4% | 1.5 ± 1.8% |
| pF(G)r7(S)r3 | | | | |

### 11.2 Two-Step Whole-Cell Reduction of Dehydrocholic Acid at the 1 L Scale

Through process control, product formation with the biocatalyst E. coli BL49 pF(G)r7(A)r3 was increased relative to the milliliter scale. For this, the biocatalyst was cultured in the 7 L bioreactor at a working volume of 4 L in the defined minimal medium. After a brief initial phase at 37° C., there was a substrate-limited exponential growth phase at 30° C. On reaching an optical density of 25-30, protein expression of the cells was induced by adding 0.5 mM IPTG. Expression was then carried out for 10 h at 25° C., before the cells were harvested by centrifugation and were stored at −20° C.

The biotransformation was set up in a 1 L bioreactor in the following reaction conditions: 70 mM DHCA, 17.7 g L⁻¹_{BTM} of the stored biocatalyst, 500 mM sodium formate, 26% (v/v) glycerol, 50 mM MgCl₂, suspended in 50 mM KPi buffer (pH 6.5). Using pH adjustment, the pH was maintained at pH 6.5 throughout the biotransformation. The variation of the bile salt concentrations as a function of time is shown in FIG. 19. After 21 h, 99.4% of product (12-keto-UDCA) had formed and only 0.6% of the by-product 3,12-diketo-UDCA was detectable.

### Reaction Example 12: Two-Step Reduction of Dehydrocholic Acid in a Single-Cell System Using a Glucose Dehydrogenase for Cofactor Regeneration

In this example, in addition to an NADPH-dependent 7β-HSDH from Collinsella aerofaciens (mutant G39A) and an NADH-dependent 3α-HSDH from Comamonas testosteroni, a GDH from Bacillus subtilis that is both NAD- and NADP-dependent for the cofactor regeneration is expressed in a whole-cell biocatalyst (single-cell system) for the two-step reduction of DHCA to 12-keto-UDCA. The nucleic acid sequence and the associated amino acid sequence of this GDH are given as SEQ ID NO:47 and SEQ ID NO:48 respectively.

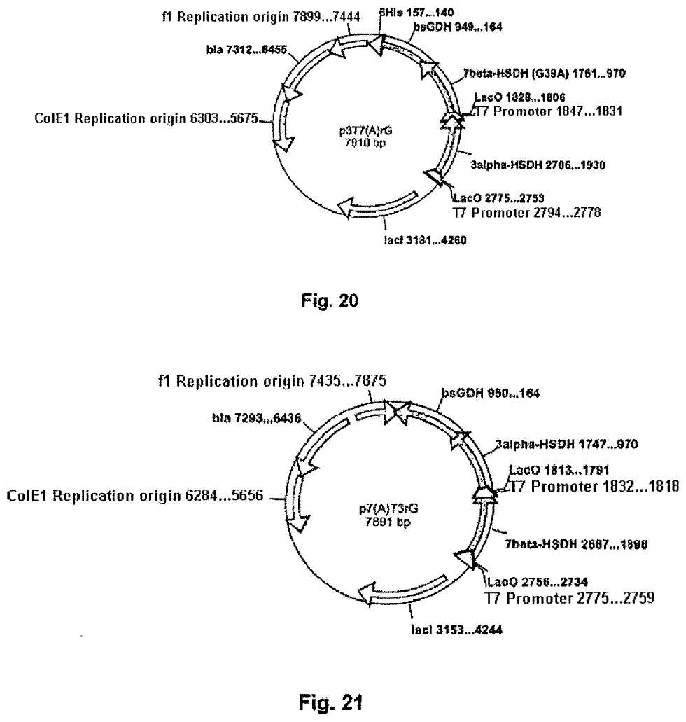

The vectors used for this bear the designations p3T7(A)rG and p7(A)T3rG and are shown in FIG. 20 and FIG. 21 respectively.

The vectors according to the invention were transformed into Escherichia coli production strains. These strains represent the whole-cell biocatalyst. The host strain used is a modified E. coli BL21(DE3) with the designation E. coli BL49 (identical to E. coli BL21(DE3) hdhA⁻KanR⁺). However, the host organism is not restricted to this host strain. The E. coli BL49 transformed with p7(A)T3rG bears the designation E. coli BL49 p7(A)T3rG, and the E. coli BL49 transformed with p3T7(A)rG bears the designation E. coli BL49 p3T7(A)rG.

With the newly produced biocatalysts, using a total of 17.7 g/L BTM biocatalysts, 100 mM DHCA can be converted to an extent of 98% to 12-keto-UDCA.

### 12.1 Two-Step Whole-Cell Reduction of Dehydrocholic Acid at the 20-mL Scale

The biocatalysts were compared by whole-cell biotransformation at the 20-mL scale. Cultivation and determination of the concentration of the substrate DHCA, of the intermediates 3,12-diketo-UDCA and 7,12-diketo-UDCA and of the product 12-keto-UDCA in the reaction mixture were carried out as described in reaction example 11.1.

The reaction mixtures for the biotransformations contained 11.8 g/L BTM whole-cell biocatalysts, 100 mM substrate (DHCA), 500 mM glucose and were suspended in 50 mM potassium formate buffer (pH 7.3). The process took place at the 20 mL scale as a batch process without pH monitoring. Perforation of the biocatalysts was not necessary. The following table shows the results of the whole-cell biotransformation described above after a process time of 24 h, showing the results of the whole-cell biotransformation with the two strains E. coli BL49 p7(A)T3rG and E. coli BL49 p3T7(A)rG. With the new biocatalysts, using 11.8 g L⁻¹_{BTM} biocatalyst, within 24 h 100 mM dehydrocholic acid (DHCA) can be converted to an extent of 46% (E. coli BL49 p3T7(A)rG) or 68% (E. coli BL49 p7(A)T3rG) to 12-keto-ursodeoxycholic acid (12-keto-UDCA). The following table shows proportions of bile salts in biotransformation batches after 24 h, using 100 mM substrate (DHCA) (batches with the two new biocatalyst strains E. coli BL49 p3T7(A)rG and E. coli BL49 p7(A)T3rG):

| | Proportions of bile salts, % | | | |
|---|---|---|---|---|
| | 12-keto- | 3,12-diketo- | 7,12-diketo- | |
| | UDCA | UDCA | UDCA | DHCA |
| E. coli BL49 | 46 ± 6% | 7 ± 1% | 7 ± 1% | 40.0 ± 2% |
| p3T7(A)rG | | | | |
| E. coli BL49 | 68 ± 19% | 15 ± 3% | 1 ± 1% | 12 ± 10% |
| p7(A)T3rG | | | | |

12.2 Two-Step Whole-Cell Reduction of Dehydrocholic Acid with Cells of the Strain E. coli BL49 p7(A)T3rG Cultured in the Bioreactor at the 20-mL Scale

The strain E. coli BL49 p7(A)T3rG was cultured in the 7 L bioreactor at a working volume of 4 L in the defined minimal medium. After a brief initial phase at 37° C. there was a substrate-limited exponential growth phase at 30° C. On reaching an optical density of 25-30, protein expression of the cells was induced by adding 0.5 mM IPTG. Expression was then carried out for 24 h at 20° C. The cells (46.7 g/L BTM) were harvested by centrifugation, resuspended in potassium phosphate buffer (pH 6.5) and stored according to the standard protocol at −20° C. At the time of harvesting, the enzyme activities were: 16.4 U/mL 7β-HSDH, 3.6 U/mL 3α-HSDH, 44.9 U/mL GDH (NADP), 18.1 U/mL GDH (NAD).

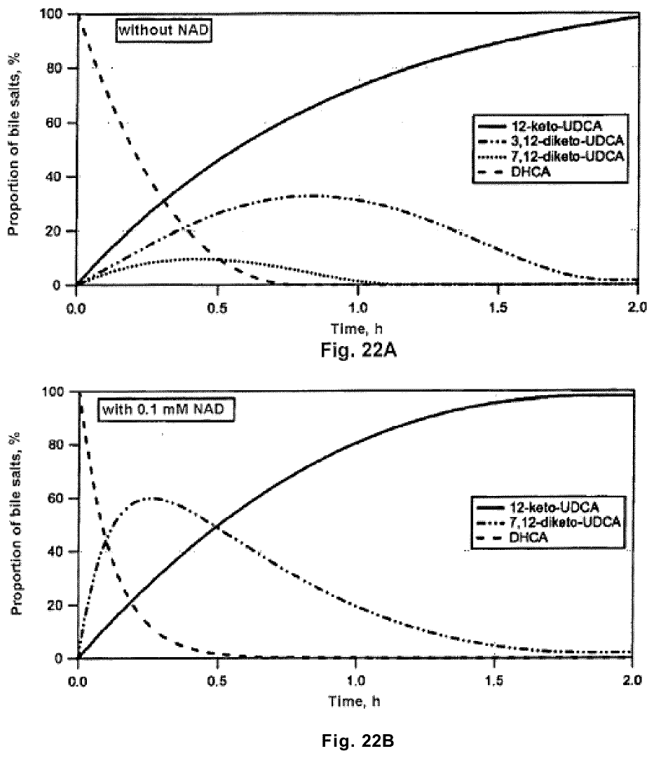

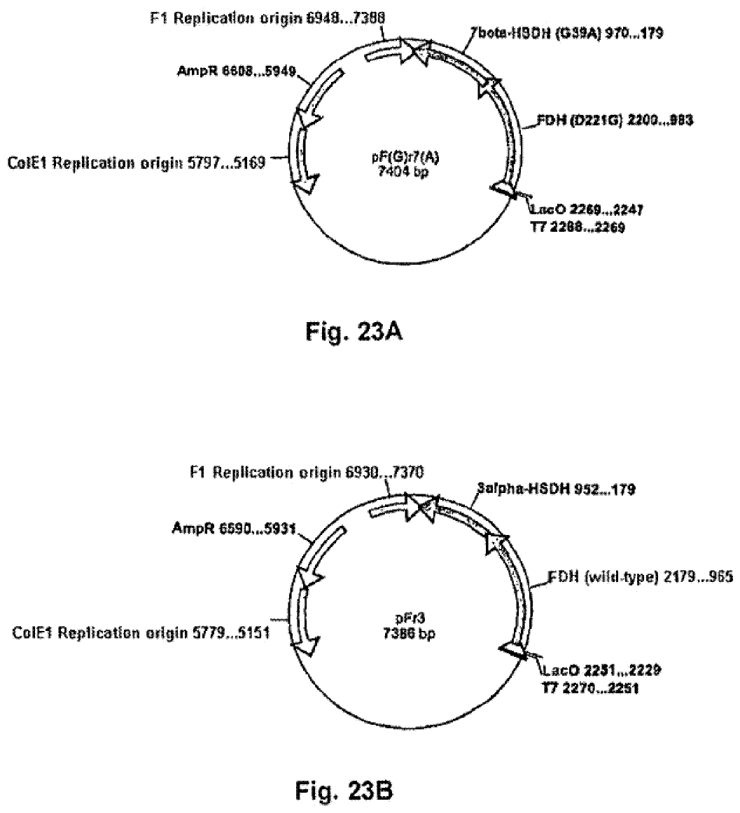

Biotransformation was set up at the 20-mL scale in the following reaction conditions: 100 mM DHCA, 17.7 g/L BTM of the stored biocatalyst, 500 mM glucose, 10 mM MgCl₂, suspended in 50 mM KPi buffer (pH 7). Using manual pH adjustment, the pH was maintained throughout the biotransformation at pH 7. The reactions were carried out either without cofactor addition or with addition of 0.1 mM NAD. The variation of the bile salt concentrations as a function of time is shown in FIG. 22. After 2 h, with and without addition of NAD, in each case ≥98% of product (12-keto-UDCA) was formed.

Using altogether 17.7 g/L BTM biocatalysts, 100 mM DHCA were converted to an extent of 98% to 12-keto-UDCA.

### Reaction Example 13: Two-Step Reduction of Dehydrocholic Acid with Parallel Use of Two Different Whole-Cell Biocatalysts

In this example, two whole-cell biocatalysts were used instead of one whole-cell biocatalyst. For this purpose, an NADP-specific FDH from Mycobacterium vaccae and an NADPH-specific 7β-HSDH from Collinsella aerofaciens were expressed in one of these biocatalysts, whereas an NAD-dependent FDH from Mycobacterium vaccae and an NADH-dependent 3α-HSDH from Comamonas testosteroni were expressed in the other biocatalyst.

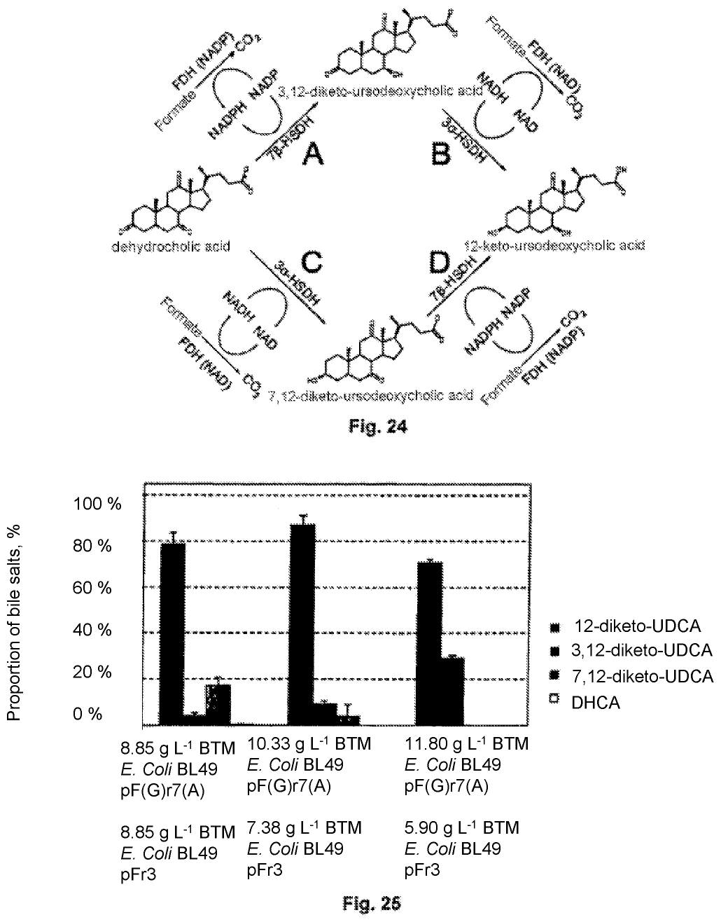

In the present example, concretely the vector pF(G)r7(A), which comprises genes for an NADP-specific FDH from Mycobacterium vaccae and for an NADPH-specific 7β-HSDH from Collinsella aerofaciens, and the vector pFr3, which comprises genes for an NAD-dependent FDH from Mycobacterium vaccae and for an NADH-dependent 3α-HSDH from Comamonas testosteroni, were used. The vectors pF(G)r7(A) and pFr3 are shown in FIGS. 23 a and b. FIG. 24 shows a reaction scheme with possible routes and products. However, the invention is not restricted to these two stated vectors, but can comprise all conceivable vectors that comprise genes for a 7β-HSDH and a suitable cofactor regeneration enzyme in combination with vectors that comprise genes for a 3α-HSDH and a suitable cofactor regeneration enzyme.

### 13.1 Whole-Cell Reduction of DHCA Using Two Biocatalysts in Different Mixture Ratios

The whole-cell reduction of DHCA to 12-keto-UDCA with different mixture ratios of the biocatalysts E. coli BL49 pF(G)r7(A) and E. coli BL49 pFr3 is shown in the following example. In this case, a total amount of biocatalyst of 17.7 g L⁻¹_{BTM} was used in each case for three batches. However, different proportions of the two biocatalysts were used in the batches. These are
8.85 g L⁻¹_{BTM} E. coli BL49 pF(G)r7(A) and 8.85 g L⁻¹_{BTM} E. coli BL49 pFr3
10.33 g L⁻¹_{BTM} E. coli BL49 pF(G)r7(A) and 7.38 g L⁻¹_{BTM} E. coli BL49 pFr3
11.80 g L⁻¹_{BTM} E. coli BL49 pF(G)r7(A) and 5.90 g L⁻¹_{BTM} E. coli BL49 pFr3
   These biocatalysis reactions were carried out at a working volume of 20 mL. Further constituents of the batches were: 70 mM DHCA, 500 mL sodium formate, 26% glycerol, 50 mM MgCl₂, suspended in 50 mL potassium phosphate buffer (pH 6.5). The reactions were carried out for 24 h. The proportions of bile salts after 24 h are shown in FIG. 25.

In the preparation with 8.85 g L⁻¹_{BTM} E. coli BL49 pF(G)r7(A) and 8.85 g L⁻¹_{BTM} E. coli BL49 pFr3, 79% 12-keto-UDCA, 4% 3,12-diketo-UDCA and 17% 7,12-diketo-UDCA are formed, in the preparation with 10.33 g L⁻¹_{BTM} E. coli BL49 pF(G)r7(A) and 7.38 g L⁻¹_{BTM} E. coli BL49 pFr3, 87% 12-keto-UDCA, 9% 3,12-diketo-UDCA and 4% 7,12-diketo-UDCA are formed and in the preparation with 11.80 g L⁻¹_{BTM} E. coli BL49 pF(G)r7(A) and 5.90 g L⁻¹_{BTM} E. coli BL49 pFr3, 71% 12-keto-UDCA and 29% 3,12-diketo-UDCA are formed. It can be seen from the proportions of the intermediates formed that in the case of the preparation with 8.85 g L⁻¹_{BTM} E. coli BL49 pF(G)r7(A) and 8.85 g L⁻¹_{BTM} E. coli BL49 pFr3 the reaction of 3α-HSDH takes place at an increased rate, as in this case a high proportion of the intermediate 7,12-diketo-UDCA is formed, whereas in the case of the preparation of 11.80 g L⁻¹_{BTM} E. coli BL49 pF(G)r7(A) and 5.90 g L⁻¹_{BTM} E. coli BL49 pFr3 the reaction of 7β-HSDH takes place at an increased rate and accordingly a high proportion of the intermediate 3,12-diketo-UDCA is formed. In the preparation with 10.33 g L⁻¹_{BTM} E. coli BL49 pF(G)r7(A) and 7.38 g L⁻¹_{BTM} E. coli BL49 pFr3, the reactions of 7β-HSDH and of 3α-HSDH are adjusted so that both occur at a roughly equal rate, as can be seen from the almost uniform formations of intermediates. As a result, the highest proportion of the product 12-keto-UDCA (87%) can be formed with this preparation.

This example shows that the rate of the individual reaction steps can be influenced by adjusting the proportions of the biocatalysts used.

### 13.1 Whole-Cell Reduction of DHCA Using Two Biocatalysts at the 1 L Scale

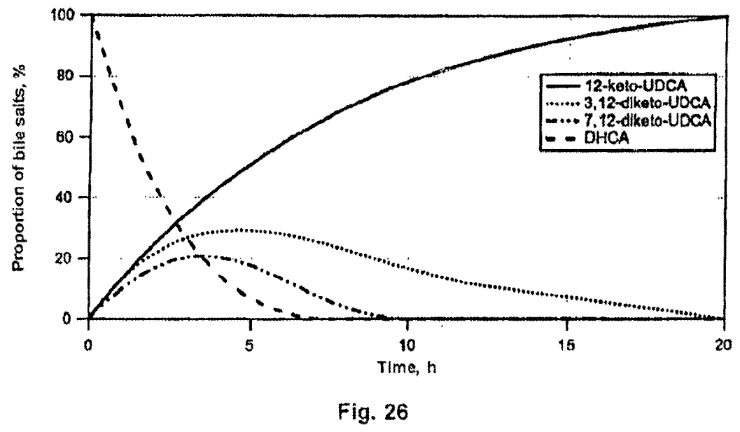

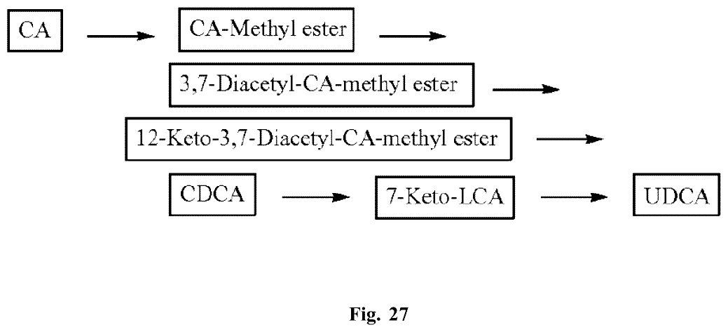

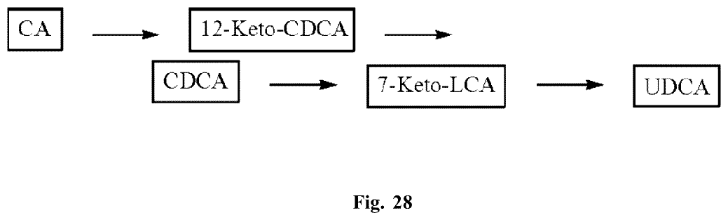

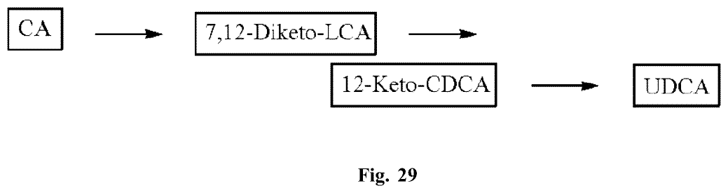

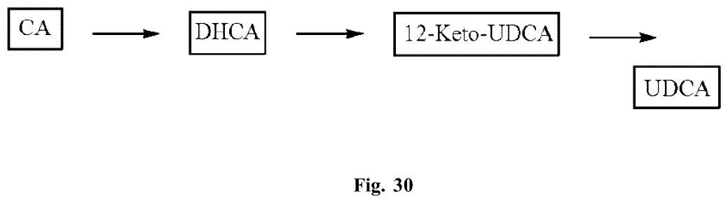

Through process control, using the biocatalysts E. coli BL49 pF(G)r7(A) and E. coli BL49 pFr3, product formation could be increased relative to the milliliter scale. The biotransformation was set up in a 1 L bioreactor in the following reaction conditions: 90 mM DHCA, with 8.85 g L⁻¹_{BTM} E. coli BL49 pF(G)r7(A) and 8.85 g L⁻¹_{BTM} E. coli BL49 pFr3, 500 mM ammonium formate, 26% (v/v) glycerol, 50 mM MgCl₂, suspended in 50 mM KPi buffer (pH 6.5). Using pH adjustment with formic acid, the pH was maintained at pH 6.5 throughout the biotransformation. The variation of the bile salt concentrations as a function of time is shown in FIG. 26. After 20 h, 99.4% product (12-keto-UDCA) had formed, and only a total of 0.6% of the intermediates 3,12-diketo-UDCA and 7,12-diketo-UDCA was detectable.

Using the biocatalysts according to the invention, with the use of a total of 17.7 g L⁻¹_{BTM} biocatalysts, 90 mM DHCA could be converted to an extent of 99.5% to 12-keto-UDCA.

### Assignment of SEQ ID NOS:

| SEQ | | |
|---|---|---|
| ID NO: | Description | Type |
| 1 | 7β-HSDH | NS |
| 2 | 7β-HSDH | AS |
| 3 | Primer S 7beta_rev_HindIII | NS |
| 4 | Primer | NS |
| 5 | 3α-HSDH (C. testosteroni) | NS |
| 6 | 3α-HSDH (C. testosteroni) | AS |
| 7 | 3α-HSDH (R. norvegicus) | NS |
| 8 | 3α-HSDH (R. norvegicus) | AS |
| 9 | Primer 7beta_mut_G39A_fwd | NS |
| 10 | Primer 7beta_mut_G39A_rev | NS |
| 11 | Primer 7beta_mut_G39S_fwd | NS |
| 12 | Primer 7beta_mut_G39S_rev | NS |
| 13 | Primer 7beta_fwd_EcoRI | NS |
| 14 | FDH D221G | NS |
| 15 | FDH D221G | AS |
| 16 | Primer 3alpha _fwd_HindIII | NS |
| 17 | Primer 3alpha_rev_NotI | NS |
| 18 | pET22a FDH D221G 7β-HSDH | NS |
| 19 | FDH D221G | AS |
| 20 | 7beta-HSDH | AS |
| 21 | pCOLA(mod) 3α-HSDH | NS |
| 22 | 3α-HSDH | AS |
| 23 | Primer fdh_for | NS |
| 24 | Primer fdh_rev | NS |
| 25 | 7α-HSDH | NS |
| 26 | 7α-HSDH | AS |
| 27 | Primer 467\|468a-IBS | NS |
| 28 | Primer 467\|468a-EBS1d | NS |
| 29 | Primer 467\|468a-EBS2 | NS |
| 30 | Primer mt1 | NS |
| 31 | Primer NI_fdh_R | NS |
| 32 | Primer 7alpha-ko-check_fwd | NS |
| 33 | Primer 7a1pha-ko-check_rev | NS |
| 34 | FDH D221G with deletion and His-Tag | NS |
| 35 | FDH D221G with deletion and His-Tag | AS |
| 36 | FDH wild type, M. vaccae | AS |
| 37 | 7β-HSDH G39D | AS |
| 38 | 7β-HSDH G39D/R40L | AS |
| 39 | 7β-HSDH G39D/R40I | AS |
| 40 | 7β-HSDH G39D/R40V | AS |
| 41 | Primer for G39D (7beta mut G39D fwd) | NS |
| 42 | Primer for G39D (7beta mut G39D rev) | NS |
| 43 | Primer for G39D R40L (7beta mut G39D R40L fwd) | NS |
| 44 | Primer 3alphamut_AntiMid_rev | NS |
| 45 | Primer for G39D R40I (7beta mut G39D R40I fwd) | NS |
| 46 | Primer 7beta mut G39D R40V fwd | NS |
| 47 | GDH, B. subtilis | NS |
| 48 | GDH B. subtilis | AS |
| 49 | Vector pFr7(D) | NS |
| 50 | Vector pFr7(DL) | NS |
| 51 | Vector pFr7(DI) | NS |
| 52 | Vector pFr7(DV) | NS |
| 53 | PCR Primer “G39D_for” | NS |
| 54 | PCR Primer “G39D_rev” | NS |
| 55 | PCR Primer “G39D/R40I_for” | NS |
| 56 | PCR Primer “G39D/R40I_rev” | NS |
| 57 | PCR Primer “R40D_for” | NS |
| 58 | PCR Primer “R40D_rev” | NS |
| 59 | PCR Primer “R40D/R41I_for” | NS |
| 60 | PCR Primer “R40D/R41I_rev” | NS |
| 61 | PCR Primer “DIN _for” | NS |
| 62 | PCR Primer “DIN_rev” | NS |
| 63 | Plasmid pFr3T7(D), Fig. 15 | NS |
| 64 | Plasmid pF(G)r7(A)r3, Fig. 17a | NS |
| 65 | Plasmid pF(G)r7(S)r3, Fig. 17b | NS |
| 66 | Plasmid p3T7(A)rG, Fig. 20 | NS |
| 67 | Plasmid p7(A)T3rG, Fig. 21 | NS |
| 68 | Plasmid pF(G)r7(A), Fig. 23a | NS |
| 69 | Plasmid pFr3, Fig. 23b | NS |

AS = amino acid sequence
NS = nucleic acid sequence

Reference is made expressly to the disclosure of the documents mentioned herein.

1. A nucleic acid molecule comprising a nucleotide sequence encoding a 7β-hydroxysteroid dehydrogenase (7β-HSDH) mutant that catalyzes at least the stereospecific enzymatic reduction of a 7-ketosteroid to the corresponding 7-hydroxysteroid, wherein the mutant has, compared to the wildtype 7β-HSDH of SEQ ID NO:2, a decreased substrate inhibition and/or an altered cofactor usage, and the mutant has, in comparison with the wildtype 7β-HSDH of SEQ ID NO:2, 1 to 15 amino acid additions, substitutions, deletions and/or inversions in the sequence motif VMVGRRE corresponding to positions 36 to 42 of SEQ ID NO:2.
